(19) United States
(12) Patent Application Publication
(10) Pub. No.: US 2023/0287364 A1
(43) Pub. Date: Sep. 14, 2023

(54) BST POLYMERASE VARIANTS

(71) Applicant: Detect, Inc., Guilford, CT (US)

(72) Inventors: David Mead, Middleton, WI (US); Yogesh Chander, Middleton, WI (US); Kurt Throckmorton, Madison, WI (US); Thomas D. Christian, Killingworth, CT (US); Brandon Neel, Guilford, CT (US)

(73) Assignee: Detect, Inc., Guildford, CT (US)

(21) Appl. No.: 18/180,838

(22) Filed: Mar. 8, 2023

Related U.S. Application Data
(60) Provisional application No. 63/317,874, filed on Mar. 8, 2022.

(51) Int. Cl. C12N 9/12; C12Q 1/70; C12Q 1/6888; C12Q 1/6876

(52) U.S. Cl. CPC C12N 9/1252; C12Y 207/07007; C12Q 1/701; C12Q 1/6888; C12Q 1/6876

(57) ABSTRACT

The present disclosure describes Bst polymerase variants which exhibit DNA-dependent DNA polymerase and reverse transcriptase activity, and methods of use thereof. The Bst polymerase variants of the disclosure may be combined with a separate enzyme having reverse transcriptase activity to amplify target RNA sequences; however, the addition of a separate enzyme having reverse transcriptase activity is not necessary for the successful amplification of RNA targets using the Bst polymerase variants of the disclosure. Target DNA sequences may also be amplified using Bst polymerase variants of the disclosure.

### RELATED APPLICATIONS

This application claims priority under 35 U.S.C. § 119(e) to U.S. Provisional Patent Application No. 63/317,874, filed Mar. 8, 2022, which is hereby incorporated by reference in its entirety.

### REFERENCE TO AN ELECTRONIC SEQUENCE LISTING

The contents of the electronic sequence listing (H096670068US01-SEQ-MKN.xml; Size: 176,275 bytes; and Date of Creation: Mar. 8, 2023) are herein incorporated by reference in their entirety.

### FIELD

The disclosure generally relates to compositions and methods for amplifying and detecting the presence of a target nucleic acid sequence.

### BACKGROUND

Loop-mediated isothermal amplification (LAMP) is an isothermal nucleic acid amplification chemistry which enables the specific amplification of target DNA sequences. LAMP has found wide appeal in recent years due to its simplicity, sensitivity, speed, and low cost, relative to other commonly-used methods of amplification (e.g., polymerase chain reaction (PCR)). Like PCR, LAMP utilizes a strand-displacing polymerase to assemble new nucleic acid molecules from a template DNA sequence of interest. For detecting target RNA sequences, typical reverse-transcription (RT) LAMP protocols include the addition of an enzyme with reverse-transcriptase activity in order to reverse transcribe the RNA to form complementary DNA (cDNA) before running the LAMP reaction. However, the addition of a further enzyme, such as a reverse transcriptase, to the LAMP/RT-LAMP protocol adds cost and complexity, and detracts from the desirable characteristics of the technique. Accordingly, improved polymerases are needed which exhibit both strand-displacement and reverse transcriptase activity.

### SUMMARY

The present disclosure relates to Bst polymerase variants which are suitable for use in an amplification method of interest. In some embodiments, said Bst polymerase variants are suitable for loop-mediated isothermal amplification (LAMP) and/or reverse transcription LAMP (RT-LAMP). In some embodiments, said Bst polymerase variants are suitable for other amplification methods.

LAMP generally refers to a DNA amplification technique originally developed by Notomi, et al., Nucl Acid Res, 28:E63 (2000), in which a target nucleic acid sequence is amplified using at least four primers through the creation of a series of stem-loop structures. Due to its use of multiple primers, LAMP may be highly specific for a target nucleic acid sequence.

As used herein, “LAMP” may encompass both LAMP and RT-LAMP. RT-LAMP combines reverse transcription with LAMP DNA amplification, reverse transcribing RNA to form complementary DNA (cDNA) before running the LAMP reaction. RT-LAMP is thus a nucleic acid amplification method to multiply specific sequences of RNA, and can be used to diagnose infectious disease caused by RNA viruses (e.g., the SARS-CoV-2 virus). In some embodiments, LAMP is RT-LAMP.

Aspects of the disclosure relate to a nucleic acid polymerase variant comprising one or more mutations relative to a wild-type Bacillus stearothermophilus (B st) nucleic acid polymerase having an amino acid sequence as shown in SEQ ID NO: 1. In some embodiments, the variant further comprises a deletion of a 5′ to 3′ exonuclease domain having an amino acid sequence as shown in SEQ ID NO: 2, relative to the wild-type Bst nucleic acid polymerase having an amino acid sequence as shown in SEQ ID NO: 1. In some embodiments, the variant further comprises an N-terminal six-histidine tag having an amino acid sequence as shown in SEQ ID NO: 3, relative to the wild-type Bst nucleic acid polymerase having an amino acid sequence as shown in SEQ ID NO: 1. In some embodiments, the variant comprises an amino acid sequence as shown in SEQ ID NO: 4.

In some embodiments, the one or more mutations comprise an amino acid substitution.

In some embodiments, the one or more mutations are made in one or more amino acid positions selected from the group consisting of: N529, K584, N602, I630, A641, I659, V663, L664, I683, T685, I691, M703, R705, Q706, F712, V715, D720, F745, D777, S787, F788, M794, A802, R825, and D832, according to the numbering as shown in SEQ ID NO: 1. In some embodiments, the one or more mutations are amino acid substitution(s) selected from the group consisting of: N529K, K584Y, N602A, N602L, I630G, A641T, I659K, V663I, L664M, I683V, T685K, I691V, M703L, R705V, Q706I, F712L, F712Y, V715M, D720A, F745Y, D777N, D777Q, S787R, F788H, F788R, M794I, A802G, R825H, and D832E, according to the numbering as shown in SEQ ID NO: 1.

In some embodiments, the variant has a single mutation, relative to SEQ ID NO: 1 and/or SEQ ID NO: 4. In some embodiments, the variant has two mutations, relative to SEQ ID NO: 1 and/or SEQ ID NO: 4. In some embodiments, the variant has three, four, five, six, seven, eight, nine, ten, eleven, twelve, thirteen, fourteen, or fifteen mutations, relative to SEQ ID NO: 1 and/or SEQ ID NO: 4.

In some embodiments, the one or more mutations are selected from the group consisting of: M794I and R825H; N529K and M794I; K584Y and M794I; N602A and D832E; N602L and D832E; I630G and M794I; A641T and M794I; I659K and M794I; R705V and M794I; F712L and M794I; F712Y and M794I; D777Q and M794I; S787R and F788R; F788R and M794I; N529K, D777Q, and M794I; K584Y, D777Q, and M794I; I630G, D777Q, and M794I; A641T, D777Q, and M794I; I659K, D777Q, and M794I; R705V, D777Q, and M794I; F712L, D777Q, and M794I; F712Y, D777Q, and M794I; D777Q, S787R, and F788R; D777Q, F788R, and M794I; S787R, F788R, and M794I; F712Y, D777Q, F788R, and M794I; D777Q, S787R, F788R, and M794I; V663I, L664M, I683V, T685K, I691V, M703L, Q706I, V715M, F745Y, and A802G; and V663I, L664M, I683V, T685K, I691V, M703L, Q706I, V715M, F745Y, M794I, and A802G according to the numbering as shown in SEQ ID NO: 1.

In some embodiments, the one or more mutations is A641T, according to the numbering as shown in SEQ ID NO: 1. In some embodiments, the one or more mutations are A641T and M794I, according to the numbering as shown in SEQ ID NO: 1. In some embodiments, the one or more mutations is D777N, according to the numbering as shown in SEQ ID NO: 1. In some embodiments, the one or more mutations are S787R and F788R, according to the numbering as shown in SEQ ID NO: 1. In some embodiments, the one or more mutations is F788R, according to the numbering as shown in SEQ ID NO: 1. In some embodiments, the one or more mutations is M794I, according to the numbering as shown in SEQ ID NO: 1. In some embodiments, the one or more mutations are M794I and R825H, according to the numbering as shown in SEQ ID NO: 1. In some embodiments, the one or more mutations are V663I, L664M, I683V, T685K, I691V, M703L, Q706I, V715M, F745Y, and A802G, according to the numbering as shown in SEQ ID NO: 1. In some embodiments, the one or more mutations are V663I, L664M, I683V, T685K, I691V, M703L, Q706I, V715M, F745Y, M794I, and A802G, according to the numbering as shown in SEQ ID NO: 1.

In some embodiments, the one or more mutations result in faster amplification of a given concentration of a target nucleic acid relative to a polymerase selected from the group consisting of: the wild-type Bst nucleic acid polymerase having an amino acid sequence as shown in SEQ ID NO: 1, a nucleic acid polymerase variant having an amino acid sequence as shown in SEQ ID NO: 4, Bst 2.0, Bst 3.0, and Geobacillus species SSD polymerase large fragment (GspSSD 2.0 LF). In some embodiments, the variant amplifies a target nucleic acid in 19 minutes or less, 18 minutes or less, 17 minutes or less, 16 minutes or less, 15 minutes or less, 14 minutes or less, 13 minutes or less, 12 minutes or less, 11 minutes or less, 10 minutes or less, 9 minutes or less, 8 minutes or less, 7 minutes or less, 6 minutes or less, 5 minutes or less, 4 minutes or less, or 3 minutes or less.

In some embodiments, the variant has increased reverse transcriptase activity for a given concentration of a target nucleic acid, relative to a polymerase selected from the group consisting of: the wild-type Bst nucleic acid polymerase having an amino acid sequence as shown in SEQ ID NO: 1, a nucleic acid polymerase variant having an amino acid sequence as shown in SEQ ID NO: 4, Bst 2.0, Bst 3.0, and Geobacillus species SSD polymerase large fragment (GspSSD 2.0 LF).

In some embodiments, the target nucleic acid is a ribonucleic acid (RNA), and amplification of the target nucleic acid occurs without a second enzyme having reverse transcriptase activity. In some embodiments, the target nucleic acid is an RNA, and amplification of the target RNA occurs with a second enzyme having reverse transcriptase activity. In some embodiments, the target RNA is RNA from MS2, SARS-CoV-2, or human ribonuclease P (RP).

In some embodiments, the target nucleic acid is a deoxyribonucleic acid (DNA). In some embodiments, the target DNA is DNA from Aeromonas.

Aspects of the disclosure relate to a nucleic acid polymerase variant comprising a polypeptide having at least 80%, at least 85%, at least 90%, at least 95%, or at least 99% sequence identity to an amino acid sequence as shown in any one of SEQ ID NOs: 6-63. In some embodiments, the nucleic acid polymerase variant has an amino acid sequence as shown in any one of SEQ ID NOs: 6-63.

Methods of using the Bst polymerase variants described herein for the detection of target nucleic acid sequences are also contemplated herein. Aspects of the disclosure thus relate to a Bst polymerase variant suitable for use in the amplification and detection of a target nucleic acid sequence.

In some embodiments, the method of detecting a target nucleic acid sequence comprises: (i) obtaining a biological sample from a subject; (ii) performing a nucleic acid amplification reaction configured to amplify the target nucleic acid sequence using a nucleic acid polymerase variant according to any embodiment of the present disclosure, and (iii) detecting the presence or absence of the target nucleic acid sequence.

In some embodiments, the target nucleic acid sequence is a DNA sequence or an RNA sequence. In some embodiments, the subject is a human, non-human primate, or mouse subject.

In some embodiments, the target nucleic acid sequence is a DNA sequence, and the nucleic acid amplification reaction comprises LAMP. In some embodiments, the target nucleic acid sequence is an RNA sequence, and the nucleic acid amplification reaction comprises RT-LAMP.

In some embodiments, the methods of the disclosure further comprise a step of adding a second enzyme having reverse transcriptase activity to the nucleic acid amplification reaction.

In some embodiments, the target nucleic acid sequence is detected using a lateral flow assay (LFA) strip, a colorimetric assay, a CRISPR/Cas method of detection, or is directly detected using hybridization.

In some embodiments, the biological sample comprises a mucus, saliva, sputum, urine, blood, or cell scraping sample. In some embodiments, the biological sample comprises a vaginal or semen sample.

Aspects of the disclosure relate to kits (e.g., test kits) for the detection of a target nucleic acid sequence comprising a nucleic acid polymerase variant according to any embodiment of the present disclosure, and methods of making such kits. In some embodiments, the kits comprise a second enzyme having reverse transcriptase activity.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D show results demonstrating the reverse transcriptase (RT) and DNA-dependent DNA polymerase activity of different Bst polymerases. FIG. 1A shows the RT activity of an optimized wild-type Bst1 sequence (SEQ ID NO: 5) and an optimized wild-type Bst59 sequence (SEQ ID NO: 4), relative to Bst 3.0 (New England Biolabs, Cat. No. M0374). The RT activity of each enzyme was determined relative to known amounts of Bst 3.0, and results are shown in Units of Bst 3.0 activity (U)/mg. FIG. 1B shows the DNA-dependent DNA polymerase activity of an optimized wild-type Bst1 sequence (SEQ ID NO: 5) and an optimized wild-type Bst59 sequence (SEQ ID NO: 4), relative to Bst 2.0 WarmStart (New England Biolabs, Cat. No. M0538). The DNA-dependent DNA polymerase activity of each enzyme was determined relative to known amounts of Bst 2.0 WarmStart, and results are shown in Units of Bst 2.0 WarmStart activity (U)/mg. FIG. 1C shows the RT activity of the following Bst polymerases, relative to Bst 3.0: an optimized wild-type Bst59 sequence (SEQ ID NO: 4), Bst59-A641T (SEQ ID NO: 11), Bst59-A641T;M794I (SEQ ID NO: 39), Bst59-D777N (SEQ ID NO: 25), Bst59-S787R;F788R (SEQ ID NO: 45), Bst59-F788R (SEQ ID NO: 29), Bst59-M794I (SEQ ID NO: 30), Bst59-M794I;R825H (alternately referred to herein as “M794IH” or “Bst59-M794IH”; SEQ ID NO: 33), Bst59-V663I;L664M;I683V;T685K; I691V;M703L;Q706I;V715M;F745Y;A802G (alternately referred to herein as “197” or “Bst59-197”; SEQ ID NO: 60), and Bst59-V663I;L664M;I683V;T685K;I691V;M703L;Q706I;V715M; F745Y;M794I;A802G (alternately referred to herein as “M794I-197” or “Bst59-M794I-197”; SEQ ID NO: 61). The RT activity of each enzyme was determined relative to known amounts of Bst 3.0, and results are shown in Units of Bst 3.0 activity (U)/mg. RT activity was assessed as in FIG. 1A. FIG. 1D shows the DNA-dependent DNA polymerase activity of the following Bst polymerases, relative to Bst 2.0 WarmStart: an optimized wild-type Bst59 sequence, Bst59-A641T, Bst59-A641T;M794I, Bst59-D777N, Bst59-S787R;F788R, Bst59-F788R, Bst59-M794I, Bst59-M794IH, Bst59-197, and Bst59-M794I-197. The DNA-dependent DNA polymerase activity of each enzyme was determined relative to known amounts of Bst 2.0 WarmStart, and results are shown in Units of Bst 2.0 WarmStart activity (U)/mg. DNA-dependent polymerase activity was assessed as in FIG. 1B.

FIGS. 2A-2C show results demonstrating the RT activity of different Bst polymerases in various buffers for an MS2 RNA target, both with and without the addition of a second enzyme having RT activity (WarmStart® RTx Reverse Transcriptase; New England Biolabs, Cat. No. M0380). Nucleic acids were amplified using a LAMP assay run at 72° C. for 30 minutes. Either RNA from the bacteriophage MS2 was spiked into the LAMP reaction mixture at concentrations ranging from 9×10⁻² to 9×10⁻⁷ μg/25 μL reaction (concentration indicated by bar color), or a no-template control was used (NTC). Time to results (TTR) is shown for each condition. FIG. 2A shows data for Bst 3.0, optimized wild-type Bst59 (control; SEQ ID NO: 4), Bst59-M794IH (SEQ ID NO: 33), and Bst59-M794I (SEQ ID NO: 30), collected using 10× ThermoPol buffer (Varigen Biosciences). FIG. 2B shows data for Bst 2.0, Bst 3.0, optimized wild-type Bst59, Bst59-M794IH, and Bst59-M794I, collected using 2× Detect buffer (Detect, Inc.). FIG. 2C shows data for Bst 2.0 and Bst 3.0, collected using IsoAmp buffers (IsoAmp for Bst 2.0; IsoAmp II for Bst 3.0; New England Biolabs, Cat. Nos. B0537S and B0374S, respectively).

FIGS. 3A-3E show results demonstrating the RT activity of different Bst polymerases in various buffers for a SARS-CoV-2 RNA target, both with and without the addition of a second enzyme having RT activity (WarmStart® RTx Reverse Transcriptase). Nucleic acids were amplified using a LAMP assay run at 64° C. for 30 minutes. Either RNA from SARS-CoV-2 was spiked into the LAMP reaction mixture at concentrations ranging from 5000 to 0 copies/25 μL reaction (concentration indicated by bar color), or a no-template control was used (NTC). TTR is shown for each condition. FIG. 3A shows data for Bst 2.0, Bst 3.0, optimized wild-type Bst59 (control; SEQ ID NO: 4), Bst59-M794IH (SEQ ID NO: 33), and Bst59-M794I (SEQ ID NO: 30), collected using 10× ThermoPol buffer and a proprietary primer mix. FIG. 3B shows data for Bst 2.0, Bst 3.0, optimized wild-type Bst59, Bst59-M794IH, and Bst59-M794I, collected using 2× Detect buffer (Detect, Inc.) and a proprietary primer mix. FIG. 3C shows data for Bst 2.0 and Bst 3.0, collected using IsoAmp buffers (IsoAmp for Bst 2.0; IsoAmp II for Bst 3.0). FIG. 3D shows data for Bst 2.0, Bst 3.0, optimized wild-type Bst59, Bst59-M794IH, and Bst59-M794I, collected using 10× ThermoPol buffer and Detect primer mix, which is specific for detection of SARS-CoV-2. FIG. 3E shows data for Bst 2.0, Bst 3.0, optimized wild-type Bst59, Bst59-M794IH, and Bst59-M794I, collected using 2× Detect buffer and Detect LAMP primer mix (Detect, Inc.).

FIG. 4 shows results demonstrating RT activity of different Bst polymerases for a human ribonuclease P (RP) gene RNA target, both with and without the addition of a second enzyme having RT activity (WarmStart® RTx Reverse Transcriptase), collected using 10× ThermoPol buffer and a primer mix as described in Curtis, et al. (A Multiplexed RT-LAMP Assay for Detection of Group M HIV-1 in Plasma or Whole Blood, J. Virol. Methods (2018)). Data is shown for Bst 2.0, Bst 3.0, optimized wild-type Bst59 (SEQ ID NO: 4), Bst59-M794IH (SEQ ID NO: 33) and Bst59-M794I (SEQ ID NO: 30). Nucleic acids were amplified using a LAMP assay run at 64° C. for 30 minutes. Either RNA from the human RP gene was spiked into the LAMP reaction mixture at concentrations ranging from 1250 to 0.0125 copies/25 μL reaction (concentration indicated by bar color), or a no-template control was used (NTC). TTR is shown for each condition.

FIGS. 5A and 5B show results demonstrating RT activity of different Bst polymerases in various buffers for an MS2 RNA target, both with and without the addition of a second enzyme having RT activity (Human Immunodeficiency Virus (HIV) RT at 75 ng per 25 μL LAMP reaction mix; Varigen Biosciences). Nucleic acids were amplified using a LAMP assay run at 72° C. for 30 minutes. Either RNA from the bacteriophage MS2 was spiked into the LAMP reaction mixture at concentrations ranging from 9×10⁻² to 9×10⁻⁷ μg/25 μL reaction mix (concentration indicated by bar color), or a no-template control was used (NTC). TTR is shown for each condition. FIG. 5A shows data for optimized wild-type Bst59 (SEQ ID NO: 4) and Bst59-M794IH (SEQ ID NO: 33) in the following buffers: ISO-004nd (OptiGene, Cat. No. ISO-004nd), 10× ThermoPol buffer, and 2× Detect buffer. FIG. 5B shows numerical data corresponding to the results shown in FIG. 5A.

FIGS. 6A and 6B show results demonstrating RT activity of different Bst polymerases in various buffers for a SARS-CoV-2 RNA target, both with and without the addition of a second enzyme having RT activity (HIV RT at 75 ng per LAMP reaction). Nucleic acids were amplified using a LAMP assay run at 64° C. for 30 minutes. Either RNA from SARS-CoV-2 was spiked into the LAMP reaction mixture at concentrations ranging from 5000 to 0.5 copies/25 μL of reaction mixture, or a no-template control was used (NTC). TTR is shown for each condition. FIG. 6A shows data for optimized wild-type Bst59 (SEQ ID NO: 4) and Bst59-M794IH (SEQ ID NO: 33) in the following buffers: ISO-004nd, 10× ThermoPol buffer, and 2× Detect buffer. FIG. 6B shows numerical data corresponding to the results shown in FIG. 6A.

FIGS. 7A-7D show results demonstrating DNA-dependent DNA polymerase activity of different Bst polymerases in various buffers for an Aeromonas DNA target. For each of FIGS. 7A-7D, data is shown for Bst 2.0, Bst 3.0, optimized wild-type Bst59 (SEQ ID NO: 4), Bst59-M794IH (SEQ ID NO: 33) and Bst59-A641T;M794I (SEQ ID NO: 39). TTR is shown for each condition. FIG. 7A shows results from a LAMP reaction run using 10× ThermoPol buffer. FIG. 7B shows results from a LAMP reaction run using 2× Detect buffer. FIG. 7C shows results from a LAMP reaction run using 10× IsoAmp buffer. FIG. 7D shows results from a LAMP reaction run using 10× IsoAmp II buffer.

FIGS. 8A-8C show results demonstrating the RT activity of Bst59-M794I (SEQ ID NO: 30) for three different RNA targets in various buffers, both with and without the addition of a second enzyme having RT activity (HIV RT at 75 ng per LAMP reaction). Nucleic acids were amplified using a LAMP assay run at 72° C. for 30 minutes for MS2 (FIG. 8A) and 64° C. for 30 minutes for both SARS-CoV-2 and human RP gene (FIGS. 8B and 8C). For each of FIGS. 8A-8C, results are shown for the following buffers: 2× Detect buffer, IsoAmp buffer, and IsoAmp II buffer. TTR is shown for each condition. FIG. 8A shows results obtained from MS2 RNA, which was spiked into the LAMP reaction mixture at concentrations ranging from 9×10⁻³ to 9×10⁻⁸ μg/25 μL reaction mix (concentration indicated by bar color). FIG. 8B shows results obtained from SARS-CoV-2 RNA, which was spiked into the LAMP reaction mixture at concentrations ranging from 5000 to 5 copies/25 μL reaction mix (concentration indicated by bar color). FIG. 8C shows results obtained from the human RP gene, which was spiked into the LAMP reaction mixture at concentrations ranging from 1250 to 1.25 copies/25 μL reaction mix (concentration indicated by bar color).

FIGS. 9A and 9B show results demonstrating the RT activity of Bst59-M794IH (SEQ ID NO: 33) for two different RNA targets in various buffers, both with and without the addition of a second enzyme having RT activity (HIV RT at 75 ng per LAMP reaction). Nucleic acids were amplified using a LAMP assay run at 72° C. for 30 minutes for MS2 (FIG. 9A) and 64° C. for 30 minutes for human RP gene (FIG. 9B). For each of FIGS. 9A and 9B, results are shown for the following buffers: 2× Detect buffer, IsoAmp buffer, and IsoAmp II buffer. TTR is shown for each condition. FIG. 9A shows results obtained from MS2 RNA, which was spiked into the LAMP reaction mixture at concentrations ranging from 9×10⁻³ to 9×10⁻⁸ μg/25 μL reaction mix (concentration indicated by bar color). FIG. 9B shows results obtained from the human RP gene, which was spiked into the LAMP reaction mixture at concentrations ranging from 1250 to 1.25 copies/25 μL reaction mix (concentration indicated by bar color).

FIGS. 10A and 10B show results demonstrating the RT activity of Bst59-A641T (SEQ ID NO: 11) for two different RNA targets in various buffers, both with and without the addition of a second enzyme having RT activity (HIV RT at 75 ng per LAMP reaction). Nucleic acids were amplified using a LAMP assay run at 64° C. for 30 minutes for both SARS-CoV-2 and human RP gene (FIGS. 10A and 10B). For each of FIGS. 10A and 10B, results are shown for the following buffers: 2× Detect buffer, IsoAmp buffer, and IsoAmp II buffer. TTR is shown for each condition. FIG. 10A shows results obtained from SARS-CoV-2 RNA, which was spiked into the LAMP reaction mixture at concentrations ranging from 5000 to 5 copies/25 μL reaction mix (concentration indicated by bar color). FIG. 10B shows results obtained from the human RP gene, which was spiked into the LAMP reaction mixture at concentrations ranging from 1250 to 1.25 copies/25 μL reaction mix (concentration indicated by bar color).

FIGS. 11A and 11B show results demonstrating the RT activity of Bst59-A641T;M794I (SEQ ID NO: 39) for two different RNA targets in various buffers, both with and without the addition of a second enzyme having RT activity (HIV RT at 75 ng per LAMP reaction). Nucleic acids were amplified using a LAMP assay run at 72° C. for 30 minutes for MS2 (FIG. 11A) and 64° C. for 30 minutes for human RP gene (FIG. 11B). For each of FIGS. 11A and 11B, results are shown for the following buffers: 2× Detect buffer, IsoAmp buffer, and IsoAmp II buffer. TTR is shown for each condition. FIG. 11A shows results obtained from MS2 RNA, which was spiked into the LAMP reaction mixture at concentrations ranging from 9×10⁻³ to 9×10⁻⁸ μg/25 μL reaction mix (concentration indicated by bar color). FIG. 11B shows results obtained from the human RP gene, which was spiked into the LAMP reaction mixture at concentrations ranging from 1250 to 1.25 copies/25 μL reaction mix (concentration indicated by bar color).

FIGS. 12A-12C show results demonstrating the RT activity of Bst59-D777N (SEQ ID NO: 25) for three different RNA targets in various buffers, both with and without the addition of a second enzyme having RT activity (HIV RT at 75 ng per LAMP reaction). Nucleic acids were amplified using a LAMP assay run at 72° C. for 30 minutes for MS2 (FIG. 12B) and 64° C. for 30 minutes for both SARS-CoV-2 and human RP gene (FIGS. 12A and 12C). For each of FIGS. 12A-12C, results are shown for the following buffers: 2× Detect buffer, IsoAmp buffer, and IsoAmp II buffer. TTR is shown for each condition. FIG. 12A shows results obtained from SARS-CoV-2 RNA, which was spiked into the LAMP reaction mixture at concentrations ranging from 5000 to 5 copies/25 μL reaction mix (concentration indicated by bar color). FIG. 12B shows results obtained from MS2 RNA, which was spiked into the LAMP reaction mixture at concentrations ranging from 9×10⁻³ to 9×10⁻⁸ μg/25 μL reaction mix (concentration indicated by bar color). FIG. 12C shows results obtained from the human RP gene, which was spiked into the LAMP reaction mixture at concentrations ranging from 1250 to 1.25 copies/25 μL reaction mix (concentration indicated by bar color).

FIGS. 13A and 13B show results demonstrating the RT activity of Bst59-197 (SEQ ID NO: 60) for two different RNA targets in various buffers, both with and without the addition of a second enzyme having RT activity (HIV RT at 75 ng per LAMP reaction). Nucleic acids were amplified using a LAMP assay run at 64° C. for 30 minutes for both SARS-CoV-2 and human RP gene (FIGS. 13A and 13B). For each of FIGS. 13A and 13B, results are shown for the following buffers: 2× Detect buffer, IsoAmp buffer, and IsoAmp II buffer. TTR is shown for each condition. FIG. 13A shows results obtained from SARS-CoV-2 RNA, which was spiked into the LAMP reaction mixture at concentrations ranging from 5000 to 5 copies/25 μL reaction mix (concentration indicated by bar color). FIG. 13B shows results obtained from the human RP gene, which was spiked into the LAMP reaction mixture at concentrations ranging from 1250 to 1.25 copies/25 μL reaction mix (concentration indicated by bar color).

FIGS. 14A and 14B show results demonstrating the RT activity of Bst59-M794I-197 (SEQ ID NO: 61) for two different RNA targets in various buffers, both with and without the addition of a second enzyme having RT activity (HIV RT at 75 ng per LAMP reaction). Nucleic acids were amplified using a LAMP assay run at 64° C. for 30 minutes for both SARS-CoV-2 and human RP gene (FIGS. 14A and 14B). For each of FIGS. 14A and 14B, results are shown for the following buffers: 2× Detect buffer, IsoAmp buffer, and IsoAmp II buffer. TTR is shown for each condition. FIG. 14A shows results obtained from SARS-CoV-2 RNA, which was spiked into the LAMP reaction mixture at concentrations ranging from 5000 to 5 copies/25 μL reaction mix (concentration indicated by bar color). FIG. 14B shows results obtained from the human RP gene, which was spiked into the LAMP reaction mixture at concentrations ranging from 1250 to 1.25 copies/25 μL reaction mix (concentration indicated by bar color).

FIG. 15 shows a plot of fluorescence signal as a function of elapsed amplification time (minutes) for RT-LAMP reactions conducted using either Bst59-A641T;M794I (SEQ ID NO: 39, referred to as “Detect Bst” in FIG. 15) or NEB Bst 2.0 with 0, 50, or 50,000 SARS-CoV-2 genome copies.

FIGS. 16A-16D show results for RT-LAMP reactions conducted with 32 primer sets using Bst59-A641T;M794I (SEQ ID NO: 39, referred to as “Detect polymerase 2.0” in FIGS. 16A-16D) or NEB Bst 2.0. FIG. 16A shows a plot of time to detection (minutes) for each primer set. FIG. 16B shows average time to detection across the 32 primer sets for 10 cp/uL or 5000 cp/uL of viral RNA. FIG. 16C shows a plot of percentage of reactions that exhibit non-specific amplification (NSA) as a function of elapsed amplification time (minutes). FIG. 16D shows average time until NSA (minutes) for Detect polymerase 2.0 and NEB Bst 2.0.

FIG. 17 shows an exemplary structure of Bst777 (SEQ ID NO: 155).

FIG. 18 shows an SDS PAGE gel showing results for C-terminal Bst59-A641T;M794I (SEQ ID NO: 156) (referred to as “C-term Bst59” in FIG. 18), N-terminal Bst59-A641T;M794I (SEQ ID NO: 39) (referred to as “N-term Bst59” in FIG. 18), and Bst777 (SEQ ID NO: 155).

FIG. 19 shows a 20% PAGE gel showing starting materials and products of primer extension assays conducted with Bst59-A641T;M794I (SEQ ID NO: 39) (referred to as “Bst59” in FIG. 19), Bst777 (SEQ ID NO: 155), and Therminator™ DNA polymerase (NEB).

FIG. 20 shows time to positive amplification result for RT-LAMP reactions conducted with Bst59-A641T;M794I (SEQ ID NO: 39) (referred to as “Bst59” in FIG. 20) and Bst777 (SEQ ID NO: 155) at three different concentrations of SARS-CoV-2 virus (1.8 cp/μL, 3.6 cp/μL, and 7.2 cp/μL) in pooled nasal matrix.

### DETAILED DESCRIPTION

### Bst Polymerase Variants

Aspects of the disclosure relate to Bst polymerase variants (e.g., “nucleic acid polymerase variants”) suitable for amplification of a target nucleic acid sequence, and methods of use thereof. As used herein, a Bst polymerase “variant” refers to a polymerase which comprises one or more amino acid mutations relative to a wild-type Bacillus stearothermophilus (Bst) nucleic acid polymerase.

The Bst polymerase variants of the present disclosure comprise one or more mutations relative to a wild-type Geobacillus sp. WCH70 (Genbank Accession No. NC_012793) Bst polymerase (SEQ ID NO: 1). Further information regarding Geobacillus sp. WCH70 can be found in Brumm, et al. (2016), Complete genome sequences of Geobacillus sp. WCH70, a thermophilic strain isolated from wood compost, Stand Genom Sci 11:33. The wild-type Geobacillus sp. WCH70 (Genbank Accession No. NC_012793) Bst polymerase (SEQ ID NO: 1) is alternatively referred to as “Bst59” herein. Concordantly, the Bst polymerase variants of the disclosure may be alternately referred to herein as “Bst59 polymerase variants.”

In some embodiments, a Bst polymerase variant comprises one or more modifications to the wild-type Bst59 nucleic acid polymerase having an amino acid sequence as shown in SEQ ID NO: 1. In some embodiments, such modifications optimize the wild-type Bst59 polymerase for certain amplification (e.g., LAMP, RT-LAMP) and/or purification (e.g., Ni-affinity column protein purification) methods. In some embodiments, the 5′ to 3′ exonuclease domain of wild-type Bst59 polymerase (Geobacillus sp. WCH70 (Genbank Accession No. NC_012793); SEQ ID NO: 1), located at the N-terminus of the wild-type protein, is deleted. In some embodiments, the 5′ to 3′ exonuclease domain of wild-type Bst59 polymerase (Geobacillus sp. WCH70 (Genbank Accession No. NC_012793); SEQ ID NO: 1) comprises an amino acid sequence as shown in SEQ ID NO: 2. In some embodiments, a six-histidine tag is added to the N-terminus of the 5′ to 3′ exonuclease domain-deficient Bst59 polymerase. In some embodiments, the six-histidine tag comprises an amino acid sequence as shown in SEQ ID NO: 3. Accordingly, in some embodiments the Bst59 polymerase variant comprises an amino acid sequence which does not comprise a 5′ to 3′ exonuclease domain and which does comprise a six-histidine tag at its N-terminus, relative to wild-type Bst59 polymerase (Geobacillus sp. WCH70 (Genbank Accession No. NC_012793); SEQ ID NO: 1). In some embodiments, said Bst59 polymerase variant comprises an amino acid sequence as shown in SEQ ID NO: 4. The Bst59 polymerase variant which comprises an amino acid sequence as shown in SEQ ID NO: 4 is referred to herein as an “optimized wild-type Bst59” polymerase.

In some embodiments, a Bst polymerase variant of the disclosure comprises one or more amino acid mutations, relative to the wild-type Bst59 polymerase sequence (SEQ ID NO: 1) and/or to the optimized wild-type Bst59 polymerase sequence (SEQ ID NO: 4). In some embodiments, a Bst polymerase variant of the disclosure comprises 1, 2, 3, 4, 5, 6, 7, 8, 9, 10, 11, 12, 13, 14, or 15 amino acid mutations, relative to the wild-type Bst59 polymerase sequence (SEQ ID NO: 1) and/or the optimized wild-type Bst59 polymerase sequence (SEQ ID NO: 4). As will be understood, an amino acid mutation may comprise the addition, deletion, or substitution (e.g., a conservative amino acid substitution, a substitution with a hydrophobic amino acid, for example A, L, or V, a substitution with a polar amino acid, for example N, S, or Q, or other amino acid substitution) of an amino acid. Any amino acid mutation described herein made be made alone or in combination, without limitation.

Throughout the disclosure, reference is made to specific amino acid positions by identifying the position of the amino acid within a reference sequence. While either the position numbering of SEQ ID NO: 1 or SEQ ID NO: 4 could be used, position numbering relative to SEQ ID NO: 1 is used throughout the disclosure for consistency. Table 3 details certain Bst59 amino acid positions/mutations which are numbered relative to both SEQ ID NO: 1 (wild-type Bst59 polymerase) and SEQ ID NO: 4 (optimized wild-type Bst59 polymerase). As can be seen in Table 3, the amino acid mutations are the same in either sequence; only the position numbers differ (due to the deletion of the 5′ to 3′ exonuclease domain from and addition of the N-terminal six-histidine tag to SEQ ID NO: 1 to produce SEQ ID NO: 4).

In some embodiments, the one or more mutations are made in one or more amino acid positions selected from the group consisting of: N529, K584, N602, I630, A641, I659, V663, L664, I683, T685, I691, M703, R705, Q706, F712, V715, D720, F745, D777, S787, F788, M794, A802, R825, and D832, according to the numbering as shown in SEQ ID NO: 1. In some embodiments, the one or more mutations is made in the amino acid position N529, according to the numbering as shown in SEQ ID NO: 1. In some embodiments, the one or more mutations is made in the amino acid position K584, according to the numbering as shown in SEQ ID NO: 1. In some embodiments, the one or more mutations is made in the amino acid position N602, according to the numbering as shown in SEQ ID NO: 1. In some embodiments, the one or more mutations is made in the amino acid position I630, according to the numbering as shown in SEQ ID NO: 1. In some embodiments, the one or more mutations is made in the amino acid position A641, according to the numbering as shown in SEQ ID NO: 1. In some embodiments, the one or more mutations is made in the amino acid position I659, according to the numbering as shown in SEQ ID NO: 1. In some embodiments, the one or more mutations is made in the amino acid position V663, according to the numbering as shown in SEQ ID NO: 1. In some embodiments, the one or more mutations is made in the amino acid position L664, according to the numbering as shown in SEQ ID NO: 1. In some embodiments, the one or more mutations is made in the amino acid position I683, according to the numbering as shown in SEQ ID NO: 1. In some embodiments, the one or more mutations is made in the amino acid position T685, according to the numbering as shown in SEQ ID NO: 1. In some embodiments, the one or more mutations is made in the amino acid position I691, according to the numbering as shown in SEQ ID NO: 1. In some embodiments, the one or more mutations is made in the amino acid position M703, according to the numbering as shown in SEQ ID NO: 1. In some embodiments, the one or more mutations is made in the amino acid position R705, according to the numbering as shown in SEQ ID NO: 1. In some embodiments, the one or more mutations is made in the amino acid position Q706, according to the numbering as shown in SEQ ID NO: 1. In some embodiments, the one or more mutations is made in the amino acid position F712, according to the numbering as shown in SEQ ID NO: 1. In some embodiments, the one or more mutations is made in the amino acid position V715, according to the numbering as shown in SEQ ID NO: 1. In some embodiments, the one or more mutations is made in the amino acid position D720, according to the numbering as shown in SEQ ID NO: 1. In some embodiments, the one or more mutations is made in the amino acid position F745, according to the numbering as shown in SEQ ID NO: 1. In some embodiments, the one or more mutations is made in the amino acid position D777, according to the numbering as shown in SEQ ID NO: 1. In some embodiments, the one or more mutations is made in the amino acid position S787, according to the numbering as shown in SEQ ID NO: 1. In some embodiments, the one or more mutations is made in the amino acid position F788, according to the numbering as shown in SEQ ID NO: 1. In some embodiments, the one or more mutations is made in the amino acid position M794, according to the numbering as shown in SEQ ID NO: 1. In some embodiments, the one or more mutations is made in the amino acid position A802, according to the numbering as shown in SEQ ID NO: 1. In some embodiments, the one or more mutations is made in the amino acid position R825, according to the numbering as shown in SEQ ID NO: 1. In some embodiments, the one or more mutations is made in the amino acid position D832, according to the numbering as shown in SEQ ID NO: 1.

In some embodiments, the one or more mutations comprise one or more amino acid substitution(s) as shown in Table 3. In some embodiments, the one or more mutations comprise one or more amino acid substitution(s) selected from: N529K, K584Y, N602A, N602L, I630G, A641T, I659K, V663I, L664M, I683V, T685K, I691V, M703L, R705V, Q706I, F712L, F712Y, V715M, D720A, F745Y, D777N, D777Q, S787R, F788H, F788R, M794I, A802G, R825H, and D832E, according to the numbering as shown in SEQ ID NO: 1. In some embodiments, the one or more mutations are one or more amino acid substitution(s) selected from the group consisting of: N529K, K584Y, N602A, N602L, I630G, A641T, I659K, V663I, L664M, I683V, T685K, I691V, M703L, R705V, Q706I, F712L, F712Y, V715M, D720A, F745Y, D777N, D777Q, S787R, F788H, F788R, M794I, A802G, R825H, and D832E, according to the numbering as shown in SEQ ID NO: 1.

In some embodiments, the one or more mutations is N529K, according to the numbering as shown in SEQ ID NO: 1. In some embodiments, the one or more mutations is K584Y, according to the numbering as shown in SEQ ID NO: 1. In some embodiments, the one or more mutations is N602A, according to the numbering as shown in SEQ ID NO: 1. In some embodiments, the one or more mutations is N602L, according to the numbering as shown in SEQ ID NO: 1. In some embodiments, the one or more mutations is I630G, according to the numbering as shown in SEQ ID NO: 1. In some embodiments, the one or more mutations is A641T, according to the numbering as shown in SEQ ID NO: 1. In some embodiments, the one or more mutations is I659K, according to the numbering as shown in SEQ ID NO: 1. In some embodiments, the one or more mutations is V663I, according to the numbering as shown in SEQ ID NO: 1. In some embodiments, the one or more mutations is L664M, according to the numbering as shown in SEQ ID NO: 1. In some embodiments, the one or more mutations is I683V, according to the numbering as shown in SEQ ID NO: 1. In some embodiments, the one or more mutations is T685K, according to the numbering as shown in SEQ ID NO: 1. In some embodiments, the one or more mutations is I691V, according to the numbering as shown in SEQ ID NO: 1. In some embodiments, the one or more mutations is M703L, according to the numbering as shown in SEQ ID NO: 1. In some embodiments, the one or more mutations is R705V, according to the numbering as shown in SEQ ID NO: 1. In some embodiments, the one or more mutations is Q706I, according to the numbering as shown in SEQ ID NO: 1. In some embodiments, the one or more mutations is F712L, according to the numbering as shown in SEQ ID NO: 1. In some embodiments, the one or more mutations is F712Y, according to the numbering as shown in SEQ ID NO: 1. In some embodiments, the one or more mutations is V715M, according to the numbering as shown in SEQ ID NO: 1. In some embodiments, the one or more mutations is D720A, according to the numbering as shown in SEQ ID NO: 1. In some embodiments, the one or more mutations is F745Y, according to the numbering as shown in SEQ ID NO: 1. In some embodiments, the one or more mutations is D777N, according to the numbering as shown in SEQ ID NO: 1. In some embodiments, the one or more mutations is D777Q, according to the numbering as shown in SEQ ID NO: 1. In some embodiments, the one or more mutations is S787R, according to the numbering as shown in SEQ ID NO: 1. In some embodiments, the one or more mutations is F788H, according to the numbering as shown in SEQ ID NO: 1. In some embodiments, the one or more mutations is F788R, according to the numbering as shown in SEQ ID NO: 1. In some embodiments, the one or more mutations is M794I, according to the numbering as shown in SEQ ID NO: 1. In some embodiments, the one or more mutations is A802G, according to the numbering as shown in SEQ ID NO: 1. In some embodiments, the one or more mutations is R825H, according to the numbering as shown in SEQ ID NO: 1. In some embodiments, the one or more mutations is D832E, according to the numbering as shown in SEQ ID NO: 1.

In some embodiments, the one or more mutations comprise one or more amino acid substitution(s) selected from: M794I and R825H; N529K and M794I; K584Y and M794I; N602A and D832E; N602L and D832E; I630G and M794I; A641T and M794I; I659K and M794I; R705V and M794I; F712L and M794I; F712Y and M794I; D777Q and M794I; S787R and F788R; F788R and M794I; N529K, D777Q, and M794I; K584Y, D777Q, and M794I; I630G, D777Q, and M794I; A641T, D777Q, and M794I; I659K, D777Q, and M794I; R705V, D777Q, and M794I; F712L, D777Q, and M794I; F712Y, D777Q, and M794I; D777Q, S787R, and F788R; D777Q, F788R, and M794I; S787R, F788R, and M794I; F712Y, D777Q, F788R, and M794I; D777Q, S787R, F788R, and M794I; V663I, L664M, I683V, T685K, I691V, M703L, Q706I, V715M, F745Y, and A802G; and V663I, L664M, I683V, T685K, I691V, M703L, Q706I, V715M, F745Y, M794I, and A802G according to the numbering as shown in SEQ ID NO: 1. In some embodiments, the one or more mutations are one or more amino acid substitution(s) selected from the group consisting of: M794I and R825H; N529K and M794I; K584Y and M794I; N602A and D832E; N602L and D832E; I630G and M794I; A641T and M794I; I659K and M794I; R705V and M794I; F712L and M794I; F712Y and M794I; D777Q and M794I; S787R and F788R; F788R and M794I; N529K, D777Q, and M794I; K584Y, D777Q, and M794I; I630G, D777Q, and M794I; A641T, D777Q, and M794I; I659K, D777Q, and M794I; R705V, D777Q, and M794I; F712L, D777Q, and M794I; F712Y, D777Q, and M794I; D777Q, S787R, and F788R; D777Q, F788R, and M794I; S787R, F788R, and M794I; F712Y, D777Q, F788R, and M794I; D777Q, S787R, F788R, and M794I; V663I, L664M, I683V, T685K, I691V, M703L, Q706I, V715M, F745Y, and A802G; and V663I, L664M, I683V, T685K, I691V, M703L, Q706I, V715M, F745Y, M794I, and A802G, according to the numbering as shown in SEQ ID NO: 1.

In some embodiments, the one or more mutations are M794I and R825H, according to the numbering as shown in SEQ ID NO: 1. In some embodiments, the one or more mutations are N529K and M794I, according to the numbering as shown in SEQ ID NO: 1. In some embodiments, the one or more mutations are K584Y and M794I, according to the numbering as shown in SEQ ID NO: 1. In some embodiments, the one or more mutations are N602A and D832E, according to the numbering as shown in SEQ ID NO: 1. In some embodiments, the one or more mutations are N602L and D832E, according to the numbering as shown in SEQ ID NO: 1. In some embodiments, the one or more mutations are I630G and M794I, according to the numbering as shown in SEQ ID NO: 1. In some embodiments, the one or more mutations are A641T and M794I, according to the numbering as shown in SEQ ID NO: 1. In some embodiments, the one or more mutations are I659K and M794I, according to the numbering as shown in SEQ ID NO: 1. In some embodiments, the one or more mutations are R705V and M794I, according to the numbering as shown in SEQ ID NO: 1. In some embodiments, the one or more mutations are F712L and M794I, according to the numbering as shown in SEQ ID NO: 1. In some embodiments, the one or more mutations are F712Y and M794I, according to the numbering as shown in SEQ ID NO: 1. In some embodiments, the one or more mutations are D777Q and M794I, according to the numbering as shown in SEQ ID NO: 1. In some embodiments, the one or more mutations are S787R and F788R, according to the numbering as shown in SEQ ID NO: 1. In some embodiments, the one or more mutations are F788R and M794I, according to the numbering as shown in SEQ ID NO: 1. In some embodiments, the one or more mutations are N529K, D777Q, and M794I, according to the numbering as shown in SEQ ID NO: 1. In some embodiments, the one or more mutations are K584Y, D777Q, and M794I, according to the numbering as shown in SEQ ID NO: 1. In some embodiments, the one or more mutations are I630G, D777Q, and M794I, according to the numbering as shown in SEQ ID NO: 1. In some embodiments, the one or more mutations are A641T, D777Q, and M794I, according to the numbering as shown in SEQ ID NO: 1. In some embodiments, the one or more mutations are I659K, D777Q, and M794I, according to the numbering as shown in SEQ ID NO: 1. In some embodiments, the one or more mutations are R705V, D777Q, and M794I, according to the numbering as shown in SEQ ID NO: 1. In some embodiments, the one or more mutations are F712L, D777Q, and M794I, according to the numbering as shown in SEQ ID NO: 1. In some embodiments, the one or more mutations are F712Y, D777Q, and M794I, according to the numbering as shown in SEQ ID NO: 1. In some embodiments, the one or more mutations are D777Q, S787R, and F788R, according to the numbering as shown in SEQ ID NO: 1. In some embodiments, the one or more mutations are D777Q, F788R, and M794I, according to the numbering as shown in SEQ ID NO: 1. In some embodiments, the one or more mutations are S787R, F788R, and M794I, according to the numbering as shown in SEQ ID NO: 1. In some embodiments, the one or more mutations are F712Y, D777Q, F788R, and M794I, according to the numbering as shown in SEQ ID NO: 1. In some embodiments, the one or more mutations are D777Q, S787R, F788R, and M794I, according to the numbering as shown in SEQ ID NO: 1. In some embodiments, the one or more mutations are V663I, L664M, I683V, T685K, I691V, M703L, Q706I, V715M, F745Y, and A802G, according to the numbering as shown in SEQ ID NO: 1. In some embodiments, the one or more mutations are V663I, L664M, I683V, T685K, I691V, M703L, Q706I, V715M, F745Y, M794I, and A802G, according to the numbering as shown in SEQ ID NO: 1.

In some embodiments, a Bst59 polymerase variant of the disclosure has an amino acid sequence as shown in Table 1. In some embodiments, a Bst59 polymerase variant of the disclosure comprises a polypeptide having at least 80% (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100%; 80-81%, 81-82%, 82-83%, 83-84%, 84-85%, 85-86%, 86-87%, 87-88%, 88-89%, 89-90%, 90-91%, 91-92%, 92-93%, 93-94%, 94-95%, 95-96%, 95-97%, 96-97%, 96-98%, 97-98%, 97-99%, 98-99%, 98-100%, or 99-100%; 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to an amino acid sequence as shown in any of SEQ ID NOs: 6-63. In some embodiments, a Bst59 polymerase variant of the disclosure has an amino acid sequence as shown in any one of SEQ ID NOs: 6-63.

In some embodiments, a Bst59 polymerase variant of the disclosure has an amino acid sequence as shown in SEQ ID NO: 6 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 7 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 8 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 9 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 10 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 11 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 12 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 13 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 14 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 15 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 16 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 17 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 18 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 19 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 20 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 21 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 22 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 23 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 24 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 25 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 26 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 27 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 28 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 29 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 30 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 31 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 32 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 33 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 34 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 35 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 36 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 37 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 38 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 39 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 40 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 41 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 42 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 43 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 44 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 45 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 46 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 47 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 48 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 49 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 50 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 51 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 52 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 53 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 54 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 55 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 56 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 57 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 58 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 59 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 60 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 61 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 62 (or an amino acid sequence having at least 80%, at least 85%, at least 90%, at least 95%, or at least 99% sequence identity thereto). In some embodiments, a Bst59 polymerase variant of the disclosure has an amino acid sequence as shown in SEQ ID NO: 63 (or an amino acid sequence having at least 80%, at least 85%, at least 90%, at least 95%, or at least 99% sequence identity thereto).

In some embodiments, a Bst59 polymerase variant comprises one or more amino acid mutations relative to the wild-type Bst59 polymerase sequence (SEQ ID NO: 1) and/or to the optimized wild-type Bst59 polymerase sequence (SEQ ID NO: 4), where the one or more mutations comprise one or more mutations found in Geobacillus sp. 777 DNA Polymerase I LF. In some cases, DNA Polymerase I LF from Geobacillus sp. 777 exhibits a relatively high resistance to inhibitors present in mucus, blood, urine, and/or semen samples, including but not limited to semenogelin I (SGI), heparin, ethanol, urea, human blood plasma, and whole blood matrix. In certain cases, introduction of one or more amino acid mutations found in Geobacillus sp. 777 DNA Polymerase I LF into a wild-type Bst59 polymerase or a Bst polymerase variant may advantageously increase resistance to one or more inhibitors present in mucus, blood, urine, and/or semen samples. Accordingly, in certain embodiments, a Bst polymerase variant described herein (e.g., a Bst polymerase variant comprising one or more mutations found in Geobacillus sp. 777 DNA Polymerase I LF) exhibits enhanced stability in a bodily fluid (e.g., mucus, blood, urine, and/or semen) and/or an organic solvent (e.g., ethanol) relative to a wild-type Bst59 polymerase. In some instances, a nucleic acid amplification reaction (e.g., a LAMP reaction, an RT-LAMP reaction) conducted with a Bst polymerase variant described herein (e.g., a Bst polymerase variant comprising one or more mutations found in Geobacillus sp. 777 DNA Polymerase I LF) on a nasal, blood, urine, or semen sample may have a shorter “time to result” than the same nucleic acid amplification reaction conducted with a wild-type Bst59 polymerase.

In some embodiments, a Bst59 polymerase variant comprises one or more amino acid mutations relative to the wild-type Bst59 polymerase sequence (SEQ ID NO: 1) and/or to the optimized wild-type Bst59 polymerase sequence (SEQ ID NO: 4), where the one or more mutations are made in one or more amino acid positions selected from the group consisting of: S299, D300, I301, D302, Y303, I305, V306, E308, S312, I313, S315, E317, L325, S327, K331, L335, F337, I339, A340, N345, I346, T350, D351, S355, S356, S357, L358, T360, Q361, E364, S367, V372, G375, I379, S381, Q385, Q388, R390, Q393, I398, S400, N404, S406, S408, T409, E410, S414, I415, T418, T422, D423, Q425, S426, I430, Q437, K438, I439, R457, Q461, D462, I464, C465, D466, Q468, E469, Y473, S474, F476, T477, D478, L481, K514, A641, Q750, K753, D755, and M794, according to the numbering as shown in SEQ ID NO: 1.

In some embodiments, a Bst59 polymerase variant comprises one or more amino acid mutations relative to the wild-type Bst59 polymerase sequence (SEQ ID NO: 1) and/or to the optimized wild-type Bst59 polymerase sequence (SEQ ID NO: 4), where the one or more amino acid mutations comprise S299A, D300K, I301M, D302A, Y303F, I305L, V306A, E308R, S312E, I313M, S315A, E317K, L325V, S327E, K331D, L335V, F337I, I339V, A340V, N345R, I346L, T350P, D351E, S355A, S356D, S357P, L358Q, T360V, Q361A, E364G, S367T, V372M, G375S, I379A, S381A, Q385K, Q388E, R390C, Q393S, I398L, S400A, N404D, S406A, S408G, T409V, E410D, S414A, I415A, T418M, T422E, D423A, Q425R, S426P, I430V, Q437R, K438A, I439V, R457W, Q461R, D462P, I464L, C465D, D466E, Q468R, E469R, Y473D, S474R, F476L, T477V, D478E, L481Q, K514R, A641T, Q750R, K753E, D755N, and/or M794I, according to the numbering as shown in SEQ ID NO: 1.

In some embodiments, a Bst59 polymerase variant comprises at least 1, at least 2, at least 3, at least 4, at least 5, at least 10, at least 15, at least 20, at least 25, at least 30, at least 40, at least 50, at least 60, at least 70, or at least 77 amino acid mutations relative to the wild-type Bst59 polymerase sequence (SEQ ID NO: 1) and/or to the optimized wild-type Bst59 polymerase sequence (SEQ ID NO: 4). In some embodiments, the Bst59 polymerase variant comprises 1 to 2, 1 to 5, 1 to 10, 1 to 15, 1 to 20, 1 to 25, 1 to 30, 1 to 40, 1 to 50, 1 to 60, 1 to 70, 1 to 77, 5 to 10, 5 to 15, 5 to 20, 5 to 25, 5 to 30, 5 to 40, 5 to 50, 5 to 60, 5 to 70, 5 to 77, 10 to 15, 10 to 20, 10 to 25, 10 to 30, 10 to 40, 10 to 50, 10 to 60, 10 to 70, 10 to 77, 20 to 30, 20 to 40, 20 to 50, 20 to 60, 20 to 70, 20 to 77, 30 to 40, 30 to 50, 30 to 60, 30 to 70, 30 to 77, 40 to 50, 40 to 60, 40 to 70, 40 to 77, 50 to 60, 50 to 70, 50 to 77, 60 to 70, or 60 to 77 amino acid mutations relative to the wild-type Bst59 polymerase sequence (SEQ ID NO: 1) and/or to the optimized wild-type Bst59 polymerase sequence (SEQ ID NO: 4).

In some embodiments, a Bst59 polymerase variant comprises a polypeptide having at least 80%, (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100%; 80-81%, 81-82%, 82-83%, 83-84%, 84-85%, 85-86%, 86-87%, 87-88%, 88-89%, 89-90%, 90-91%, 91-92%, 92-93%, 93-94%, 94-95%, 95-96%, 95-97%, 96-97%, 96-98%, 97-98%, 97-99%, 98-99%, 98-100%, or 99-100%; 80%, 81%, 82%, 83%, 84%, 85%, 86%, 87%, 88%, 89%, 90%, 91%, 92%, 93%, 94%, 95%, 96%, 97%, 98%, 99%, or 100%) sequence identity to an amino acid sequence as shown in SEQ ID NO: 155. A Bst polymerase variant having an amino acid sequence as shown in SEQ ID NO: 155 may be referred to herein as “Bst777.”

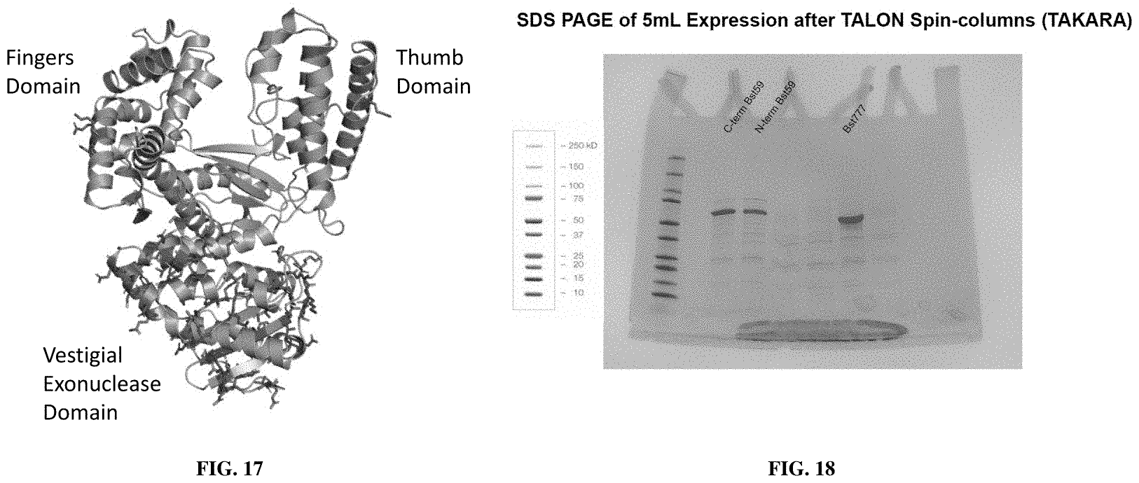

In some cases, a Bst59 polymerase or a variant thereof comprises a fingers domain, a thumb domain, a palm domain, and/or a vestigial 3′-5′ exonuclease domain. As an illustrative example, FIG. 17 shows an exemplary structure of Bst777 (SEQ ID NO: 155), with the fingers domain shown in the upper left, the thumb domain shown in the upper right, and vestigial 3′-5′ exonuclease domain shown in the bottom center.

In some embodiments, a Bst polymerase variant comprises one or more mutations in the fingers domain. In certain embodiments, the Bst polymerase variant comprises 1, 2, 3, 4, 5, 6, 7, 8, 9, or 10 mutations in the fingers domain. In some instances, the Bst polymerase variant has 4 mutations in the fingers domain. In some embodiments, a Bst polymerase variant comprises one or more mutations in the thumb domain. In certain embodiments, the Bst polymerase variant comprises 1, 2, 3, 4, 5, 6, 7, 8, 9, or 10 mutations in the thumb domain. In some instances, the Bst polymerase variant has 2 mutations in the thumb domain. In some embodiments, a Bst polymerase variant comprises one or more mutations in the vestigial 3′-5′ exonuclease domain. In some embodiments, the Bst polymerase variant comprises 1 to 5, 1 to 10, 1 to 15, 1 to 20, 1 to 30, 1 to 40, 1 to 50, 1 to 60, 1 to 70, 1 to 71, 5 to 10, 5 to 15, 5 to 20, 5 to 30, 5 to 40, 5 to 50, 5 to 60, 5 to 70, 5 to 71, 10 to 15, 10 to 20, 10 to 30, 10 to 40, 10 to 50, 10 to 60, 10 to 70, 10 to 71, 20 to 30, 20 to 40, 20 to 50, 20 to 60, 20 to 70, 20 to 71, 30 to 40, 30 to 50, 30 to 60, 30 to 70, 30 to 71, 40 to 50, 40 to 60, 40 to 70, 40 to 71, 50 to 60, 50 to 70, 50 to 71, 60 to 71, or 60 to 71 mutations in the vestigial 3′-5′ exonuclease domain. In some instances, the Bst polymerase variant has 71 mutations in the vestigial 3′-5′ exonuclease domain. In certain embodiments, a Bst polymerase variant (e.g., Bst777) comprises 4 mutations in the fingers domain, 2 mutations in the thumb domain, and 71 mutations in the vestigial exonuclease domain.

In some cases, a Bst59 polymerase comprises one or more highly conserved regions and/or one or more less conserved regions. Highly conserved and less conserved regions may be identified by aligning DNA polymerase sequences of Geobacillus sp. WCH70 and several related species, including but not limited to P. yumthangensis, P. thermantarcticus, Bacillus alveayuensis, Gacillus sp. G (2006), Bacillus caldolyticus, S. thermophilus, A. tepidamans, T. altinsuensis, N. thermocopriae, G. thermoleovorans, and Geobacillus sp. 777. In some cases, a Bst polymerase variant comprises one or more mutations in one or more less conserved regions. In certain embodiments, a Bst polymerase variant comprises at least 1, at least 2, at least 3, at least 4, at least 5, at least 10, at least 15, or at least 20 mutations in one or more less conserved regions. In certain cases, the Bst polymerase variant has 1 to 2, 1 to 3, 1 to 4, 1 to 5, 1 to 10, 1 to 15, 1 to 20, 2 to 5, 2 to 10, 2 to 15, 2 to 20, 5 to 10, 5 to 15, 5 to 20, or 15 to 20 mutations in one or more less conserved regions. In some cases, a Bst polymerase variant comprises relatively few mutations in one or more highly conserved regions. In certain embodiments, a Bst polymerase variant comprises 5 or fewer mutations, 4 or fewer mutations, 3 or fewer mutations, 2 or fewer mutations, 1 mutation, or no mutations in one or more highly conserved regions. In some cases, limiting or avoiding mutations in one or more highly conserved regions may maintain or enhance DNA replication and fidelity.

TABLE 1
Bst sequences of the disclosure

| SEQ | | |
|---|---|---|
| ID NO | Sequence | Description |
| 1 | MKKKLVLIDGNSIAYRAFFALPLLHNDKGIHTNAVYGFTMMLMKILEEEKPTHMLVAFDAGKTTFRHKTFEEYKGG | Wild-type |
| | RQQTPPELSEQFPLLRELLDAYRIRFYELENYEADDIIGTLCTKAEKEGFEVKVISGDRDLTQLASDHVTVDITKK | Bst59 |
| | GITDVESYTPETVQEKYGLTPEQIIDLKGLMGDKSDNIPGVPGIGEKTALKLLKEFGTIENILDSIEQVSGNKLRE | (Geobacillus |
| | NLAKYRDIALMSKQLATILRDAPVDLSLEDIEYNGYDVDKVIALFKELGFNSLLEKMAAPQGEEETEVALSDIDYT | sp. WCH70 |
| | IVDEVTESILSDEAALVVEVLESNYHKAPILGFAIANEHGNFFIRTDTALSSSLFTQWLEDESKKKSVFDGKRAIV | (Genbank |
| | SLKWQGIQLRGVQFDLLIASYLLNPSQSTEDVASIAKTKQYTDVQSDEAIYGKGAKQKIPDEPVLAEHLVRKAAAI | Accession No. |
| | RALEQDFICDLQENEQYSLFTDLELPLSSILAEMEFTGVKIDVKRLKEMGEELTEQLKEIEQEIYELAGQEFNINS | NC_012793)) |
| | PKQLGVILFEKLQLPVLKKTKTGYSTSAEVLEKLAPQHEIVEKILHYRQLGKLQSTYIEGLLKVVHHDTNKVHTIF | |
| | NQALTQTGRLSSTEPNLQNIPIRLEEGRKIRQAFVPSEPDWVIFSADYSQIELRVLAHIANDENLIDAFRHDLDIH | |
| | TKTAMDIFHVNEDEVTPNMRRQAKAVNFGIVYGISDYGLSQNLNITRKEASEFIKRYFEIFPGVKQYMKDIVQEAK | |
| | QKGYVTTLLHRRRYLPDITSRNFNLRSFAERTAMNTPIQGSAADIIKKAMIDLSNRLKKENMKARMLLQVHDELIL | |
| | EAPKEEVERLQQIVPEVMENAVQLRVPLKVDYHFGPTWYDAK | |
| 2 | MKKKLVLIDGNSIAYRAFFALPLLHNDKGIHTNAVYGFTMMLMKILEEEKPTHMLVAFDAGKTTFRHKTFEEYKGG | 5′ to 3′ |
| | RQQTPPELSEQFPLLRELLDAYRIRFYELENYEADDIIGTLCTKAEKEGFEVKVISGDRDLTQLASDHVTVDITKK | exonuclease |
| | GITDVESYTPETVQEKYGLTPEQIIDLKGLMGDKSDNIPGVPGIGEKTALKLLKEFGTIENILDSIEQVSGNKLRE | domain (wild- |
| | NLAKYRDIALMSKQLATILRDAPVDLSLEDIEYNGYDVDKVIALFKELGFNSLLEKMAAPQGEE | type Bst59 |
| | | (Geobacillus |
| | | sp. WCH70 |
| | | Accession No. |
| | | NC_012793); |
| | | SEQ ID NO: 1) |
| 3 | MHHHHHHG | N-terminal |
| | | six-histidine |
| | | tag |
| 4 | MHHHHHHGETEVALSDIDYTIVDEVTESILSDEAALVVEVLESNYHKAPILGFAIANEHGNFFIRTDTALSSSLFT | Optimized |
| | QWLEDESKKKSVFDGKRAIVSLKWQGIQLRGVQFDLLIASYLLNPSQSTEDVASIAKTKQYTDVQSDEAIYGKGAK | wild-type |
| | QKIPDEPVLAEHLVRKAAAIRALEQDFICDLQENEQYSLFTDLELPLSSILAEMEFTGVKIDVKRLKEMGEELTEQ | (Bst59 (e.g., |
| | LKEIEQEIYELAGQEFNINSPKQLGVILFEKLQLPVLKKTKTGYSTSAEVLEKLAPQHEIVEKILHYRQLGKLQST | Geobacillus |
| | YIEGLLKVVHHDTNKVHTIFNQALTQTGRLSSTEPNLQNIPIRLEEGRKIRQAFVPSEPDWVIFSADYSQIELRVL | sp. WCH70 |
| | AHIANDENLIDAFRHDLDIHTKTAMDIFHVNEDEVTPNMRRQAKAVNFGIVYGISDYGLSQNLNITRKEASEFIKR | ((Genbank |
| | YFEIFPGVKQYMKDIVQEAKQKGYVTTLLHRRRYLPDITSRNFNLRSFAERTAMNTPIQGSAADIIKKAMIDLSNR | Accession No. |
| | LKKENMKARMLLQVHDELILEAPKEEVERLQQIVPEVMENAVQLRVPLKVDYHFGPTWYDAK | NC_012793); |
| | | SEQ ID NO: 1) |
| | | comprising an |
| | | N-terminal |
| | | six-histidine |
| | | tag (SEQ ID |
| | | NO: 2) and a |
| | | deletion of a |
| | | 5′ to 3′ |
| | | exonuclease |
| | | domain (SEQ |
| | | ID NO: 3)) |
| 5 | MHHHHHHAEGEKPLEEMEFAIVDVITEEMLADKAALVVEVMEENYHDAPIVGIALVNEHGRFFMRPETALADSQFL | Optimized |
| | AWLADETKKKSMFDAKRAVVALKWKGIELRGVAFDLLLAAYLLNPAQDAGDIAAVAKMKQYEAVRSDEAVYGKGVK | wild-type |
| | RSLPDEQTLAEHLVRKAAAIWALEQPFMDDLRNNEQDQLLTKLEQPLAAILAEMEFTGVNVDTKRLEQMGSELAEQ | Bst1 (e.g., |
| | LRAIEQRIYELAGQEFNINSPKQLGVILFEKLQLPVLKKTKTGYSTSADVLEKLAPHHEIVENILHYRQLGKLQST | Geobacillus |
| | YIEGLLKVVRPDTGKVHTMFNQALTQTGRLSSAEPNLQNIPIRLEEGRKIRQAFVPSEPDWLIFAADYSQIELRVL | sp. G11MC16 |
| | AHIADDDNLIEAFQRDLDIHTKTAMDIFHVSEEEVTANMRRQAKAVNFGIVYGISDYGLAQNLNITRKEAAEFIER | comprising an |
| | YFASFPGVKQYMENIVQEAKQKGYVTTLLHRRRYLPDITSRNFNVRSFAERTAMNTPIQGSAADIIKKAMIDLAAR | N-terminal |
| | LKEEQLQARLLLQVHDELILEAPKEEIERLCELVPEVMEQAVTLRVPLKVDYHYGPTWYDAK | six-histidine |
| | | tag and a |
| | | deletion of a |
| | | 5′ to 3′ |
| | | exonuclease |
| | | domain) |
| 6 | MHHHHHHGETEVALSDIDYTIVDEVTESILSDEAALVVEVLESNYHKAPILGFAIANEHGNFFIRTDTALSSSLFT | Bst59-N529K |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFKINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 7 | MHHHHHHGETEVALSDIDYTIVDEVTESILSDEAALVVEVLESNYHKAPILGFAIANEHGNFFIRTDTALSSSLFT | Bst59-K584Y |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGYLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 8 | MHHHHHHGETEVALSDIDYTIVDEVTESILSDEAALVVEVLESNYHKAPILGFAIANEHGNFFIRTDTALSSSLFT | Bst59-N602A |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTAKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 9 | MHHHHHHGETEVALSDIDYTIVDEVTESILSDEAALVVEVLESNYHKAPILGFAIANEHGNFFIRTDTALSSSLFT | Bst59-N602L |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTLKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 10 | MHHHHHHGETEVALSDIDYTIVDEVTESILSDEAALVVEVLESNYHKAPILGFAIANEHGNFFIRTDTALSSSLFT | Bst59-I630G |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPGRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 11 | MHHHHHHGETEVALSDIDYTIVDEVTESILSDEAALVVEVLESNYHKAPILGFAIANEHGNFFIRTDTALSSSLFT | Bst59-A641T |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQTFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 12 | MHHHHHHGETEVALSDIDYTIVDEVTESILSDEAALVVEVLESNYHKAPILGFAIANEHGNFFIRTDTALSSSLFT | Bst59-I659K |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQKELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 13 | MHHHHHHGETEVALSDIDYTIVDEVTESILSDEAALVVEVLESNYHKAPILGFAIANEHGNFFIRTDTALSSSLFT | Bst59-V663I |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRIL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 14 | MHHHHHHGETEVALSDIDYTIVDEVTESILSDEAALVVEVLESNYHKAPILGFAIANEHGNFFIRTDTALSSSLFT | Bst59-L664M |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVM | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 15 | MHHHHHHGETEVALSDIDYTIVDEVTESILSDEAALVVEVLESNYHKAPILGFAIANEHGNFFIRTDTALSSSLFT | Bst59-I683V |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDVHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 16 | MHHHHHHGETEVALSDIDYTIVDEVTESILSDEAALVVEVLESNYHKAPILGFAIANEHGNFFIRTDTALSSSLFT | Bst59-T685K |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHKKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 17 | MHHHHHHGETEVALSDIDYTIVDEVTESILSDEAALVVEVLESNYHKAPILGFAIANEHGNFFIRTDTALSSSLFT | Bst59-I691V |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDVFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 18 | MHHHHHHGETEVALSDIDYTIVDEVTESILSDEAALVVEVLESNYHKAPILGFAIANEHGNFFIRTDTALSSSLFT | Bst59-M703L |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDVFHVNEDEVTPNLRRQAKAVNFGIVYGISDYGLSQNLNITRKEASEFIKR | |
| | YFEIFPGVKQYMKDIVQEAKQKGYVTTLLHRRRYLPDITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 19 | MHHHHHHGETEVALSDIDYTIVDEVTESILSDEAALVVEVLESNYHKAPILGFAIANEHGNFFIRTDTALSSSLFT | Bst59-R705V |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRVQAKAVNFGIVYGISDYGLSQNLNITRKEASEFIKR | |
| | YFEIFPGVKQYMKDIVQEAKQKGYVTTLLHRRRYLPDITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 20 | MHHHHHHGETEVALSDIDYTIVDEVTESILSDEAALVVEVLESNYHKAPILGFAIANEHGNFFIRTDTALSSSLFT | Bst59-Q706I |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDVFHVNEDEVTPNMRRIAKAVNFGIVYGISDYGLSQNLNITRKEASEFIKR | |
| | YFEIFPGVKQYMKDIVQEAKQKGYVTTLLHRRRYLPDITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 21 | MHHHHHHGETEVALSDIDYTIVDEVTESILSDEAALVVEVLESNYHKAPILGFAIANEHGNFFIRTDTALSSSLFT | Bst59-F712L |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNLGIVYGISDYGLSQNLNITRKEASEFIKR | |
| | YFEIFPGVKQYMKDIVQEAKQKGYVTTLLHRRRYLPDITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 22 | MHHHHHHGETEVALSDIDYTIVDEVTESILSDEAALVVEVLESNYHKAPILGFAIANEHGNFFIRTDTALSSSLFT | Bst59-F712Y |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNYGIVYGISDYGLSQNLNITRKEASEFIKR | |
| | YFEIFPGVKQYMKDIVQEAKQKGYVTTLLHRRRYLPDITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 23 | MHHHHHHGETEVALSDIDYTIVDEVTESILSDEAALVVEVLESNYHKAPILGFAIANEHGNFFIRTDTALSSSLFT | Bst59-V715M |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDVFHVNEDEVTPNMRRQAKAVNFGIMYGISDYGLSQNLNITRKEASEFIKR | |
| | YFEIFPGVKQYMKDIVQEAKQKGYVTTLLHRRRYLPDITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 24 | MHHHHHHGETEVALSDIDYTIVDEVTESILSDEAALVVEVLESNYHKAPILGFAIANEHGNFFIRTDTALSSSLFT | Bst59-D720A |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISAYGLSQNLNITRKEASEFIKR | |
| | YFEIFPGVKQYMKDIVQEAKQKGYVTTLLHRRRYLPDITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 25 | MHHHHHHGETEVALSDIDYTIVDEVTESILSDEAALVVEVLESNYHKAPILGFAIANEHGNFFIRTDTALSSSLFT | Bst59-D777N |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPNITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 26 | MHHHHHHGETEVALSDIDYTIVDEVTESILSDEAALVVEVLESNYHKAPILGFAIANEHGNFFIRTDTALSSSLFT | Bst59-D777Q |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPQITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 27 | MHHHHHHGETEVALSDIDYTIVDEVTESILSDEAALVVEVLESNYHKAPILGFAIANEHGNFFIRTDTALSSSLFT | Bst59-S787R |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRRFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 28 | MHHHHHHGETEVALSDIDYTIVDEVTESILSDEAALVVEVLESNYHKAPILGFAIANEHGNFFIRTDTALSSSLFT | Bst59-F788H |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSHAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 29 | MHHHHHHGETEVALSDIDYTIVDEVTESILSDEAALVVEVLESNYHKAPILGFAIANEHGNFFIRTDTALSSSLFT | Bst59-F788R |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSRAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 30 | MHHHHHHGETEVALSDIDYTIVDEVTESILSDEAALVVEVLESNYHKAPILGFAIANEHGNFFIRTDTALSSSLFT | Bst59-M794I |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSFAERTAINTPIQGSAADIIKKAMIDLSNR | |
| | LKKENMKARMLLQVHDELILEAPKEEVERLQQIVPEVMENAVQLRVPLKVDYHFGPTWYDAK | |
| 31 | MHHHHHHGETEVALSDIDYTIVDEVTESILSDEAALVVEVLESNYHKAPILGFAIANEHGNFFIRTDTALSSSLFT | Bst59-R825H |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSFAERTAMNTPIQGSAADIIKKAMIDLSNR | |
| | LKKENMKAHMLLQVHDELILEAPKEEVERLQQIVPEVMENAVQLRVPLKVDYHFGPTWYDAK | |
| 32 | MHHHHHHGETEVALSDIDYTIVDEVTESILSDEAALVVEVLESNYHKAPILGFAIANEHGNFFIRTDTALSSSLFT | Bst59-D832E |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSFAERTAMNTPIQGSAADIIKKAMIDLSNR | |
| | LKKENMKARMLLQVHEELILEAPKEEVERLQQIVPEVMENAVQLRVPLKVDYHFGPTWYDAK | |
| 33 | MHHHHHHGETEVALSDIDYTIVDEVTESILSDEAALVVEVLESNYHKAPILGFAIANEHGNFFIRTDTALSSSLFT | Bst59-M794I; |
| | QWLEDESKKKSVFDGKRAIVSLKWQGIQLRGVQFDLLIASYLLNPSQSTEDVASIAKTKQYTDVQSDEAIYGKGAK | R825H |
| | QKIPDEPVLAEHLVRKAAAIRALEQDFICDLQENEQYSLFTDLELPLSSILAEMEFTGVKIDVKRLKEMGEELTEQ | (alternately |
| | LKEIEQEIYELAGQEFNINSPKQLGVILFEKLQLPVLKKTKTGYSTSAEVLEKLAPQHEIVEKILHYRQLGKLQST | referred to |
| | YIEGLLKVVHHDTNKVHTIFNQALTQTGRLSSTEPNLQNIPIRLEEGRKIRQAFVPSEPDWVIFSADYSQIELRVL | herein as |
| | AHIANDENLIDAFRHDLDIHTKTAMDIFHVNEDEVTPNMRRQAKAVNFGIVYGISDYGLSQNLNITRKEASEFIKR | “M794H” or |
| | YFEIFPGVKQYMKDIVQEAKQKGYVTTLLHRRRYLPDITSRNFNLRSFAERTAINTPIQGSAADIIKKAMIDLSNR | “Bst59- |
| | LKKENMKAHMLLQVHDELILEAPKEEVERLQQIVPEVMENAVQLRVPLKVDYHFGPTWYDAK | M794IH”) |
| 34 | MHHHHHHGETEVALSDIDYTIVDEVTESILSDEAALVVEVLESNYHKAPILGFAIANEHGNFFIRTDTALSSSLFT | Bst59-N529K; |
| | QWLEDESKKKSVFDGKRAIVSLKWQGIQLRGVQFDLLIASYLLNPSQSTEDVASIAKTKQYTDVQSDEAIYGKGAK | M794I |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFKINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSFAERTAINTPIQGSAADIIKKAMIDLSNR | |
| | LKKENMKARMLLQVHDELILEAPKEEVERLQQIVPEVMENAVQLRVPLKVDYHFGPTWYDAK | |
| 35 | MHHHHHHGETEVALSDIDYTIVDEVTESILSDEAALVVEVLESNYHKAPILGFAIANEHGNFFIRTDTALSSSLFT | Bst59-K584Y; |
| | QWLEDESKKKSVFDGKRAIVSLKWQGIQLRGVQFDLLIASYLLNPSQSTEDVASIAKTKQYTDVQSDEAIYGKGAK | M794I |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGYLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSFAERTAINTPIQGSAADIIKKAMIDLSNR | |
| | LKKENMKARMLLQVHDELILEAPKEEVERLQQIVPEVMENAVQLRVPLKVDYHFGPTWYDAK | |
| 36 | MHHHHHHGETEVALSDIDYTIVDEVTESILSDEAALVVEVLESNYHKAPILGFAIANEHGNFFIRTDTALSSSLFT | Bst59-N602A; |
| | QWLEDESKKKSVFDGKRAIVSLKWQGIQLRGVQFDLLIASYLLNPSQSTEDVASIAKTKQYTDVQSDEAIYGKGAK | D832E |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTAKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSFAERTAMNTPIQGSAADIIKKAMIDLSNR | |
| | LKKENMKARMLLQVHEELILEAPKEEVERLQQIVPEVMENAVQLRVPLKVDYHFGPTWYDAK | |
| 37 | MHHHHHHGETEVALSDIDYTIVDEVTESILSDEAALVVEVLESNYHKAPILGFAIANEHGNFFIRTDTALSSSLFT | Bst59-N602L; |
| | QWLEDESKKKSVFDGKRAIVSLKWQGIQLRGVQFDLLIASYLLNPSQSTEDVASIAKTKQYTDVQSDEAIYGKGAK | D832E |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTLKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSFAERTAMNTPIQGSAADIIKKAMIDLSNR | |
| | LKKENMKARMLLQVHEELILEAPKEEVERLQQIVPEVMENAVQLRVPLKVDYHFGPTWYDAK | |
| 38 | MHHHHHHGETEVALSDIDYTIVDEVTESILSDEAALVVEVLESNYHKAPILGFAIANEHGNFFIRTDTALSSSLFT | Bst59-I630G; |
| | QWLEDESKKKSVFDGKRAIVSLKWQGIQLRGVQFDLLIASYLLNPSQSTEDVASIAKTKQYTDVQSDEAIYGKGAK | M794I |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPGRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSFAERTAINTPIQGSAADIIKKAMIDLSNR | |
| | LKKENMKARMLLQVHDELILEAPKEEVERLQQIVPEVMENAVQLRVPLKVDYHFGPTWYDAK | |
| 39 | MHHHHHHGETEVALSDIDYTIVDEVTESILSDEAALVVEVLESNYHKAPILGFAIANEHGNFFIRTDTALSSSLFT | Bst59-A641T; |
| | QWLEDESKKKSVFDGKRAIVSLKWQGIQLRGVQFDLLIASYLLNPSQSTEDVASIAKTKQYTDVQSDEAIYGKGAK | M794I |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQTFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSFAERTAINTPIQGSAADIIKKAMIDLSNR | |
| | LKKENMKARMLLQVHDELILEAPKEEVERLQQIVPEVMENAVQLRVPLKVDYHFGPTWYDAK | |
| 40 | MHHHHHHGETEVALSDIDYTIVDEVTESILSDEAALVVEVLESNYHKAPILGFAIANEHGNFFIRTDTALSSSLFT | Bst59-1659K; |
| | QWLEDESKKKSVFDGKRAIVSLKWQGIQLRGVQFDLLIASYLLNPSQSTEDVASIAKTKQYTDVQSDEAIYGKGAK | M794I |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQKELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSFAERTAINTPIQGSAADIIKKAMIDLSNR | |
| | LKKENMKARMLLQVHDELILEAPKEEVERLQQIVPEVMENAVQLRVPLKVDYHFGPTWYDAK | |
| 41 | MHHHHHHGETEVALSDIDYTIVDEVTESILSDEAALVVEVLESNYHKAPILGFAIANEHGNFFIRTDTALSSSLFT | Bst59-R705V; |
| | QWLEDESKKKSVFDGKRAIVSLKWQGIQLRGVQFDLLIASYLLNPSQSTEDVASIAKTKQYTDVQSDEAIYGKGAK | M794I |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRVQAKAVNFGIVYGISDYGLSQNLNITRKEASEFIKR | |
| | YFEIFPGVKQYMKDIVQEAKQKGYVTTLLHRRRYLPDITSRNFNLRSFAERTAINTPIQGSAADIIKKAMIDLSNR | |
| | LKKENMKARMLLQVHDELILEAPKEEVERLQQIVPEVMENAVQLRVPLKVDYHFGPTWYDAK | |
| 42 | MHHHHHHGETEVALSDIDYTIVDEVTESILSDEAALVVEVLESNYHKAPILGFAIANEHGNFFIRTDTALSSSLFT | Bst59-F712L; |
| | QWLEDESKKKSVFDGKRAIVSLKWQGIQLRGVQFDLLIASYLLNPSQSTEDVASIAKTKQYTDVQSDEAIYGKGAK | M794I |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNLGIVYGISDYGLSQNLNITRKEASEFIKR | |
| | YFEIFPGVKQYMKDIVQEAKQKGYVTTLLHRRRYLPDITSRNFNLRSFAERTAINTPIQGSAADIIKKAMIDLSNR | |
| | LKKENMKARMLLQVHDELILEAPKEEVERLQQIVPEVMENAVQLRVPLKVDYHFGPTWYDAK | |
| 43 | MHHHHHHGETEVALSDIDYTIVDEVTESILSDEAALVVEVLESNYHKAPILGFAIANEHGNFFIRTDTALSSSLFT | Bst59-F712Y; |
| | QWLEDESKKKSVFDGKRAIVSLKWQGIQLRGVQFDLLIASYLLNPSQSTEDVASIAKTKQYTDVQSDEAIYGKGAK | M794I |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNYGIVYGISDYGLSQNLNITRKEASEFIKR | |
| | YFEIFPGVKQYMKDIVQEAKQKGYVTTLLHRRRYLPDITSRNFNLRSFAERTAINTPIQGSAADIIKKAMIDLSNR | |
| | LKKENMKARMLLQVHDELILEAPKEEVERLQQIVPEVMENAVQLRVPLKVDYHFGPTWYDAK | |
| 44 | MHHHHHHGETEVALSDIDYTIVDEVTESILSDEAALVVEVLESNYHKAPILGFAIANEHGNFFIRTDTALSSSLFT | Bst59-D777Q; |
| | QWLEDESKKKSVFDGKRAIVSLKWQGIQLRGVQFDLLIASYLLNPSQSTEDVASIAKTKQYTDVQSDEAIYGKGAK | M794I |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPQITSRNFNLRSFAERTAINTPIQGSAADIIKKAMIDLSNR | |
| | LKKENMKARMLLQVHDELILEAPKEEVERLQQIVPEVMENAVQLRVPLKVDYHFGPTWYDAK | |
| 45 | MHHHHHHGETEVALSDIDYTIVDEVTESILSDEAALVVEVLESNYHKAPILGFAIANEHGNFFIRTDTALSSSLFT | Bst59-S787R; |
| | QWLEDESKKKSVFDGKRAIVSLKWQGIQLRGVQFDLLIASYLLNPSQSTEDVASIAKTKQYTDVQSDEAIYGKGAK | F788R |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRRRAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 46 | MHHHHHHGETEVALSDIDYTIVDEVTESILSDEAALVVEVLESNYHKAPILGFAIANEHGNFFIRTDTALSSSLFT | Bst59-F788R; |
| | QWLEDESKKKSVFDGKRAIVSLKWQGIQLRGVQFDLLIASYLLNPSQSTEDVASIAKTKQYTDVQSDEAIYGKGAK | M794I |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSRAERTAINTPIQGSAADIIKKAMIDLSNR | |
| | LKKENMKARMLLQVHDELILEAPKEEVERLQQIVPEVMENAVQLRVPLKVDYHFGPTWYDAK | |
| 47 | MHHHHHHGETEVALSDIDYTIVDEVTESILSDEAALVVEVLESNYHKAPILGFAIANEHGNFFIRTDTALSSSLFT | Bst59-N529K; |
| | QWLEDESKKKSVFDGKRAIVSLKWQGIQLRGVQFDLLIASYLLNPSQSTEDVASIAKTKQYTDVQSDEAIYGKGAK | D777Q; M794I |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFKINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPQITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 48 | MHHHHHHGETEVALSDIDYTIVDEVTESILSDEAALVVEVLESNYHKAPILGFAIANEHGNFFIRTDTALSSSLFT | Bst59-K584Y; |
| | QWLEDESKKKSVFDGKRAIVSLKWQGIQLRGVQFDLLIASYLLNPSQSTEDVASIAKTKQYTDVQSDEAIYGKGAK | D777Q; M794I |
| | QKIPDEPVLAEHLVRKAAAIRALEQDFICDLQENEQYSLFTDLELPLSSILAEMEFTGVKIDVKRLKEMGEELTEQ | |
| | LKEIEQETYELAGQEFNINSPKQLGVILFEKLQLPVLKKTKTGYSTSAEVLEKLAPQHEIVEKILHYRQLGYLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPQITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 49 | MHHHHHHGETEVALSDIDYTIVDEVTESILSDEAALVVEVLESNYHKAPILGFAIANEHGNFFIRTDTALSSSLFT | Bst59-1630G; |
| | QWLEDESKKKSVFDGKRAIVSLKWQGIQLRGVQFDLLIASYLLNPSQSTEDVASIAKTKQYTDVQSDEAIYGKGAK | D777Q; M794I |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPGRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPQITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 50 | MHHHHHHGETEVALSDIDYTIVDEVTESILSDEAALVVEVLESNYHKAPILGFAIANEHGNFFIRTDTALSSSLFT | Bst59-A641T; |
| | QWLEDESKKKSVFDGKRAIVSLKWQGIQLRGVQFDLLIASYLLNPSQSTEDVASIAKTKQYTDVQSDEAIYGKGAK | D777Q; M794I |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQTFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPQITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 51 | MHHHHHHGETEVALSDIDYTIVDEVTESILSDEAALVVEVLESNYHKAPILGFAIANEHGNFFIRTDTALSSSLFT | Bst59-1659K; |
| | QWLEDESKKKSVFDGKRAIVSLKWQGIQLRGVQFDLLIASYLLNPSQSTEDVASIAKTKQYTDVQSDEAIYGKGAK | D777Q; M794I |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQKELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPQITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 52 | MHHHHHHGETEVALSDIDYTIVDEVTESILSDEAALVVEVLESNYHKAPILGFAIANEHGNFFIRTDTALSSSLFT | Bst59-R705V; |
| | QWLEDESKKKSVFDGKRAIVSLKWQGIQLRGVQFDLLIASYLLNPSQSTEDVASIAKTKQYTDVQSDEAIYGKGAK | D777Q; M794I |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRVQAKAVNFGIVYGISDYGLSQNLNITRKEASEFIKR | |
| | YFEIFPGVKQYMKDIVQEAKQKGYVTTLLHRRRYLPQITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 53 | MHHHHHHGETEVALSDIDYTIVDEVTESILSDEAALVVEVLESNYHKAPILGFAIANEHGNFFIRTDTALSSSLFT | Bst59-F712L; |
| | QWLEDESKKKSVFDGKRAIVSLKWQGIQLRGVQFDLLIASYLLNPSQSTEDVASIAKTKQYTDVQSDEAIYGKGAK | D777Q; M794I |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNLGIVYGISDYGLSQNLNITRKEASEFIKR | |
| | YFEIFPGVKQYMKDIVQEAKQKGYVTTLLHRRRYLPQITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 54 | MHHHHHHGETEVALSDIDYTIVDEVTESILSDEAALVVEVLESNYHKAPILGFAIANEHGNFFIRTDTALSSSLFT | Bst59-F712Y; |
| | QWLEDESKKKSVFDGKRAIVSLKWQGIQLRGVQFDLLIASYLLNPSQSTEDVASIAKTKQYTDVQSDEAIYGKGAK | D777Q; M794I |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNYGIVYGISDYGLSQNLNITRKEASEFIKR | |
| | YFEIFPGVKQYMKDIVQEAKQKGYVTTLLHRRRYLPQITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 55 | MHHHHHHGETEVALSDIDYTIVDEVTESILSDEAALVVEVLESNYHKAPILGFAIANEHGNFFIRTDTALSSSLFT | Bst59-D777Q; |
| | QWLEDESKKKSVFDGKRAIVSLKWQGIQLRGVQFDLLIASYLLNPSQSTEDVASIAKTKQYTDVQSDEAIYGKGAK | S787R; F788R |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPQITSRNFNLRRFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 56 | MHHHHHHGETEVALSDIDYTIVDEVTESILSDEAALVVEVLESNYHKAPILGFAIANEHGNFFIRTDTALSSSLFT | Bst59-D777Q; |
| | QWLEDESKKKSVFDGKRAIVSLKWQGIQLRGVQFDLLIASYLLNPSQSTEDVASIAKTKQYTDVQSDEAIYGKGAK | F788R; M794I |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPQITSRNFNLRSRAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 57 | MHHHHHHGETEVALSDIDYTIVDEVTESILSDEAALVVEVLESNYHKAPILGFAIANEHGNFFIRTDTALSSSLFT | Bst59-S787R; |
| | QWLEDESKKKSVFDGKRAIVSLKWQGIQLRGVQFDLLIASYLLNPSQSTEDVASIAKTKQYTDVQSDEAIYGKGAK | F788R; M794I |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRRRAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 58 | MHHHHHHGETEVALSDIDYTIVDEVTESILSDEAALVVEVLESNYHKAPILGFAIANEHGNFFIRTDTALSSSLFT | Bst59-F712Y; |
| | QWLEDESKKKSVFDGKRAIVSLKWQGIQLRGVQFDLLIASYLLNPSQSTEDVASIAKTKQYTDVQSDEAIYGKGAK | D777Q; F788R; |
| | QKIPDEPVLAEHLVRKAAAIRALEQDFICDLQENEQYSLFTDLELPLSSILAEMEFTGVKIDVKRLKEMGEELTEQ | M794I |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNYGIVYGISDYGLSQNLNITRKEASEFIKR | |
| | YFEIFPGVKQYMKDIVQEAKQKGYVTTLLHRRRYLPQITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 59 | MHHHHHHGETEVALSDIDYTIVDEVTESILSDEAALVVEVLESNYHKAPILGFAIANEHGNFFIRTDTALSSSLFT | Bst59-D777Q; |
| | QWLEDESKKKSVFDGKRAIVSLKWQGIQLRGVQFDLLIASYLLNPSQSTEDVASIAKTKQYTDVQSDEAIYGKGAK | S787R; F788R; |
| | QKIPDEPVLAEHLVRKAAAIRALEQDFICDLQENEQYSLFTDLELPLSSILAEMEFTGVKIDVKRLKEMGEELTEQ | M794I |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPQITSRNFNLRRFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 60 | MHHHHHHGETEVALSDIDYTIVDEVTESILSDEAALVVEVLESNYHKAPILGFAIANEHGNFFIRTDTALSSSLFT | Bst59-V663I; |
| | QWLEDESKKKSVFDGKRAIVSLKWQGIQLRGVQFDLLIASYLLNPSQSTEDVASIAKTKQYTDVQSDEAIYGKGAK | L664M; I683V; |
| | QKIPDEPVLAEHLVRKAAAIRALEQDFICDLQENEQYSLFTDLELPLSSILAEMEFTGVKIDVKRLKEMGEELTEQ | T685K; I691V; |
| | LKEIEQEIYELAGQEFNINSPKQLGVILFEKLQLPVLKKTKTGYSTSAEVLEKLAPQHEIVEKILHYRQLGKLQST | M703L; Q706I; |
| | YIEGLLKVVHHDTNKVHTIFNQALTQTGRLSSTEPNLQNIPIRLEEGRKIRQAFVPSEPDWVIFSADYSQIELRIM | V715M; F745Y; |
| | AHIANDENLIDAFRHDLDVHKKTAMDVFHVNEDEVTPNLRRIAKAVNFGIMYGISDYGLSQNLNITRKEASEFIKR | A820G |
| | YFEIYPGVKQYMKDIVQEAKQKGYVTTLLHRRRYLPDITSRNFNLRSFAERTAMNTPIQGSGADIIKKAMIDLSNR | (alternately |
| | LKKENMKARMLLQVHDELILEAPKEEVERLQQIVPEVMENAVQLRVPLKVDYHFGPTWYDAK | referred to |
| | | herein as |
| | | “197” or |
| | | “Bst59-197”) |
| 61 | MHHHHHHGETEVALSDIDYTIVDEVTESILSDEAALVVEVLESNYHKAPILGFAIANEHGNFFIRTDTALSSSLFT | Bst59-V663I; |
| | QWLEDESKKKSVFDGKRAIVSLKWQGIQLRGVQFDLLIASYLLNPSQSTEDVASIAKTKQYTDVQSDEAIYGKGAK | L664M; I683V; |
| | QKIPDEPVLAEHLVRKAAAIRALEQDFICDLQENEQYSLFTDLELPLSSILAEMEFTGVKIDVKRLKEMGEELTEQ | T685K; I691V; |
| | LKEIEQEIYELAGQEFNINSPKQLGVILFEKLQLPVLKKTKTGYSTSAEVLEKLAPQHEIVEKILHYRQLGKLQST | M703L; Q706I; |
| | YIEGLLKVVHHDTNKVHTIFNQALTQTGRLSSTEPNLQNIPIRLEEGRKIRQAFVPSEPDWVIFSADYSQIELRIM | V715M; F745Y; |
| | AHIANDENLIDAFRHDLDVHKKTAMDVFHVNEDEVTPNLRRIAKAVNFGIMYGISDYGLSQNLNITRKEASEFIKR | M794I; A802G |
| | YFEIYPGVKQYMKDIVQEAKQKGYVTTLLHRRRYLPDITSRNFNLRSFAERTAINTPIQGSGADIIKKAMIDLSNR | (alternately |
| | LKKENMKARMLLQVHDELILEAPKEEVERLQQIVPEVMENAVQLRVPLKVDYHFGPTWYDAK | referred to |
| | | herein as |
| | | “M794I-197” or |
| | | “Bs759-M794I- |
| | | 197”) |
| 62 | MHHHHHHGETEVALSDIDYTIVDEVTESILSDEAALVVEVLESNYHKAPILGFAIANEHGNFFIRTDTALSSSLFT | Bst59-F745Y |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIYPGVKQYMKDIVQEAKQKGYVTTLLHRRRYLPDITSRNFNLRSFAERTAMNTPIQGSAADIIKKAMIDLSNR | |
| | LKKENMKARMLLQVHDELILEAPKEEVERLQQIVPEVMENAVQLRVPLKVDYHFGPTWYDAK | |
| 63 | MHHHHHHGETEVALSDIDYTIVDEVTESILSDEAALVVEVLESNYHKAPILGFAIANEHGNFFIRTDTALSSSLFT | Bst59-A802G |
| | QWLEDESKKKSVFDGKRAIVSLKWQGIQLRGVQFDLLIASYLLNPSQSTEDVASIAKTKQYTDVQSDEAIYGKGAK | |
| | QKIPDEPVLAEHLVRKAAAIRALEQDFICDLQENEQYSLFTDLELPLSSILAEMEFTGVKIDVKRLKEMGEELTEQ | |
| | LKEIEQEIYELAGQEFNINSPKQLGVILFEKLQLPVLKKTKTGYSTSAEVLEKLAPQHEIVEKILHYRQLGKLQST | |
| | YIEGLLKVVHHDTNKVHTIFNQALTQTGRLSSTEPNLQNIPIRLEEGRKIRQAFVPSEPDWVIFSADYSQIELRVL | |
| | AHIANDENLIDAFRHDLDIHTKTAMDIFHVNEDEVTPNMRRQAKAVNFGIVYGISDYGLSQNLNITRKEASEFIKR | |
| | YFEIFPGVKQYMKDIVQEAKQKGYVTTLLHRRRYLPDITSRNFNLRSFAERTAMNTPIQGSGADIIKKAMIDLSNR | |
| | LKKENMKARMLLQVHDELILEAPKEEVERLQQIVPEVMENAVQLRVPLKVDYHFGPTWYDAK | |
| 155 | MHHHHHHGSESPSSEEEKPLAKMAFTLADRVTEEMLADKAALVVEVVEENYHDAPIVGIAVVNEHGRFFLRPETAL | Bst777 |
| | ADPQFVAWLGDETKKKSMFDSKRAAVALKWKGIELCGVSFDLLLAAYLLDPAQGVDDVAAAAKMKQYEAVRPDEAV | |
| | YGKGAKRAVPDEPVLAEHLVRKAAAIWALERPFLDELRRNEQDRLLVELEQPLSSILAEMEFTGVKIDVKRLKEMG | |
| | EELTEQLREIEQEIYELAGQEFNINSPKQLGVILFEKLQLPVLKKTKTGYSTSAEVLEKLAPQHEIVEKILHYRQL | |
| | GKLQSTYIEGLLKVVHHDTNKVHTIFNQALTQTGRLSSTEPNLQNIPIRLEEGRKIRQTFVPSEPDWVIFSADYSQ | |
| | IELRVLAHIANDENLIDAFRHDLDIHTKTAMDIFHVNEDEVTPNMRRQAKAVNFGIVYGISDYGLSQNLNITRKEA | |
| | SEFIKRYFEIFPGVKRYMENIVQEAKQKGYVTTLLHRRRYLPDITSRNFNLRSFAERTAINTPIQGSAADIIKKAM | |
| | IDLSNRLKKENMKARMLLQVHDELILEAPKEEVERLQQIVPEVMENAVQLRVPLKVDYHFGPTWYDAK | |
| 156 | MGETEVALSDIDYTIVDEVTESILSDEAALVVEVLESNYHKAPILGFAIANEHGNFFIRTDTALSSSLFTQWLEDE | Bst59-A641T; |
| | SKKKSVFDGKRAIVSLKWQGIQLRGVQFDLLIASYLLNPSQSTEDVASIAKTKQYTDVQSDEAIYGKGAKQKIPDE | M794I (SEQ ID |
| | PVLAEHLVRKAAAIRALEQDFICDLQENEQYSLFTDLELPLSSILAEMEFTGVKIDVKRLKEMGEELTEQLKEIEQ | NO: 39) |
| | EIYELAGQEFNINSPKQLGVILFEKLQLPVLKKTKTGYSTSAEVLEKLAPQHEIVEKILHYRQLGKLQSTYIEGLL | comprising a |
| | KVVHHDTNKVHTIFNQALTQTGRLSSTEPNLQNIPIRLEEGRKIRQTFVPSEPDWVIFSADYSQIELRVLAHIAND | C-terminal |
| | ENLIDAFRHDLDIHTKTAMDIFHVNEDEVTPNMRRQAKAVNFGIVYGISDYGLSQNLNITRKEASEFIKRYFEIFP | six-histidine |
| | GVKQYMKDIVQEAKQKGYVTTLLHRRRYLPDITSRNFNLRSFAERTAINTPIQGSAADIIKKAMIDLSNRLKKENM | tag instead of |
| | KARMLLQVHDELILEAPKEEVERLQQIVPEVMENAVQLRVPLKVDYHFGPTWYDAKHHHHHH | an N-terminal |
| | | six-histidine |
| | | tag |

### Methods of Amplification

Aspects of the disclosure relate to Bst59 polymerase variants suitable for use in a method of amplification which amplifies nucleic acid sequence(s) of interest.

In some embodiments, the Bst59 polymerase variants disclosed herein may be useful in a method of amplification which comprises LAMP or RT-LAMP. As used herein, “LAMP” may encompass both LAMP and RT-LAMP. As will be understood, LAMP employs a primer set, specific to the nucleic acid of interest, comprising four primers, the F3 primer, B3 primer, forward inner primer (FIP), and backward inner primer (BIP). Additionally, two optional primers, a forward loop (LF) primer and/or a backward loop (LB) primer, can also be included in the LAMP reaction. In certain cases, the loop primers target cyclic structures formed during amplification and can accelerate amplification. One or both of the LF and LB primers may be included; the addition of both loop primers can significantly accelerate LAMP. Accordingly, in some embodiments, a Bst59 polymerase variant of the disclosure is used in combination with a LAMP primer set which is specific for the nucleic acid sequence of interest (see, e.g., Table 2).

As will be understood, LAMP is an isothermal method of amplification which often takes place at a temperature between about 60° C. to 68° C. However, certain nucleic acid sequences of interest may be optimally amplified at temperatures which may be outside of this range. For example, bacteriophage MS2 (Emesvirus zinderi), a commonly-used RNA template, may be amplified using RT-LAMP at a temperature of about 72° C. Further, although LAMP often amplifies a target nucleic acid sequence in a period of time having a duration of about 20 minutes to 1 hour (see, e.g., Notomi, et al., Nucl Acid Res (2000), 28:12, e63), experimentally optimal conditions can lead to amplification which occurs in significantly less time.

In some embodiments, the Bst59 polymerase variants disclosed herein may be useful in other methods of nucleic acid amplification (e.g., isothermal nucleic acid amplification methods other than LAMP). Non-limiting examples of suitable methods of nucleic acid amplification include strand displacement amplification (SDA), helicase-dependent amplification (HDA), recombinase polymerase amplification (RPA), rolling circle amplification (RCA), nicking enzyme amplification reaction (NEAR), nucleic acid sequence-based amplification (NASBA), isothermal multiple displacement amplification (IMDA), rolling circle amplification (RCA), transcription mediated amplification (TMA), signal mediated amplification of RNA technology (SMART), single primer isothermal amplification (SPIA), circular helicase-dependent amplification (cHDA), and whole genome amplification (WGA). In some embodiments, the Bst59 polymerase variants disclosed herein may be useful in polymerase chain reaction (PCR). It should be understood that each reference to an amplification method herein may encompass the amplification method and/or the reverse transcription amplification method (e.g., “PCR” may encompass PCR and/or RT-PCR), unless context dictates otherwise.

In some embodiments, one or more mutations as described herein result in faster amplification of a given concentration of a target nucleic acid relative to a control polymerase. Said control polymerase may be a wild-type or naturally-occurring polymerase, or may be a commercially-available polymerase. In some embodiments, the control polymerase is selected from the group consisting of: the wild-type Bst nucleic acid polymerase having an amino acid sequence as shown in SEQ ID NO: 1, a nucleic acid polymerase variant having an amino acid sequence as shown in SEQ ID NO: 4, Bst 2.0 (New England Biolabs, Cat. No. M0357), Bst 3.0 (New England Biolabs, Cat. No. M0374), and Geobacillus species SSD polymerase large fragment (GspSSD 2.0 LF). In some embodiments, the Geobacillus species SSD polymerase large fragment (GspSSD 2.0 LF) is comprised within a buffer, such as, for example, ISO-004nd (OptiGene, Cat. No. ISO-004nd).

In some embodiments, a Bst59 polymerase variant as described herein amplifies a target nucleic acid in 19 minutes or less, 18 minutes or less, 17 minutes or less, 16 minutes or less, 15 minutes or less, 14 minutes or less, 13 minutes or less, 12 minutes or less, 11 minutes or less, 10 minutes or less, 9 minutes or less, 8 minutes or less, 7 minutes or less, 6 minutes or less, 5 minutes or less, 4 minutes or less, or 3 minutes or less. In some embodiments, a target nucleic acid (e.g., a SARS-CoV-2 nucleic acid sequence) is amplified using a Bst59 polymerase variant in 20 minutes or less, 21 minutes or less, 22 minutes or less, 23 minutes or less, 24 minutes or less, 25 minutes or less, 26 minutes or less, 27 minutes or less, 28 minutes or less, 29 minutes or less, 30 minutes or less, 31 minutes or less, 32 minutes or less, 33 minutes or less, 34 minutes or less, 35 minutes or less, 36 minutes or less, 37 minutes or less, 38 minutes or less, 39 minutes or less, 40 minutes or less, 41 minutes or less, 42 minutes or less, 43 minutes or less, 44 minutes or less, 45 minutes or less, 46 minutes or less, 47 minutes or less, 48 minutes or less, 49 minutes or less, 50 minutes or less, 51 minutes or less, 52 minutes or less, 53 minutes or less, 54 minutes or less, 55 minutes or less, 56 minutes or less, 57 minutes or less, 58 minutes or less, 59 minutes or less, or 60 minutes or less (e.g., 1 hour or less). In some embodiments, a target nucleic acid is amplified using a Bst59 polymerase variant in about 3-5 minutes, about 4-6 minutes, about 5-7 minutes, about 6-8 minutes, about 7-9 minutes, about 8-10 minutes, about 9-11 minutes, about 10-12 minutes, about 11-13 minutes, about 12-14 minutes, about 13-15 minutes, about 14-16 minutes, about 15-17 minutes, about 16-18 minutes, about 17-19 minutes, about 18-20 minutes, about 19-21 minutes, about 20-22 minutes, about 21-23 minutes, about 22-24 minutes, or about 23-25 minutes. In some embodiments, a target nucleic acid is amplified using a Bst59 polymerase variant in about 15-16 minutes, about 16-17 minutes, about 17-18 minutes, about 18-19 minutes, about 19-20 minutes, about 20-21 minutes, about 21-22 minutes, about 22-23 minutes, about 23-24 minutes, or about 24-25 minutes.

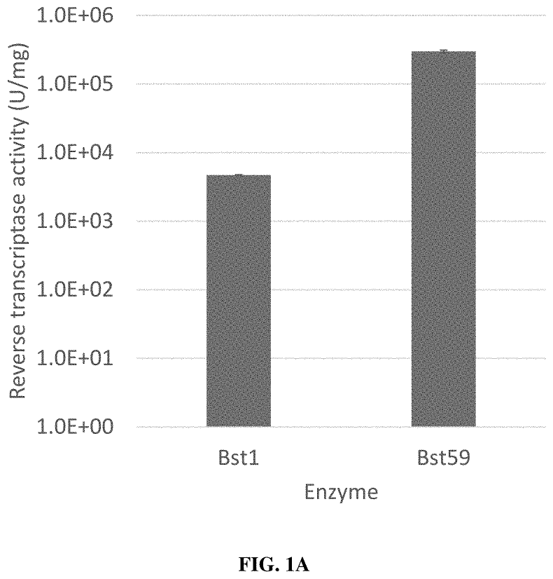

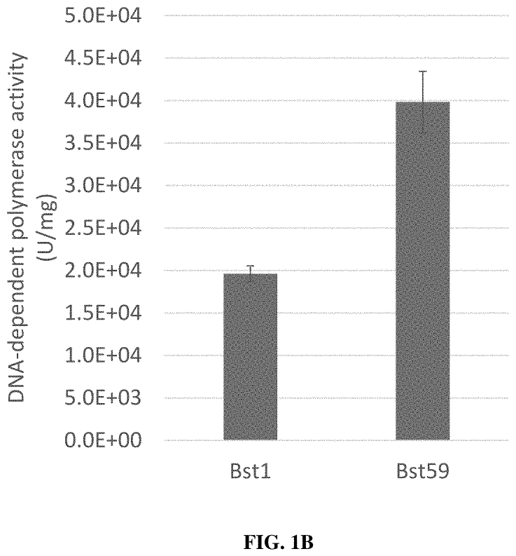

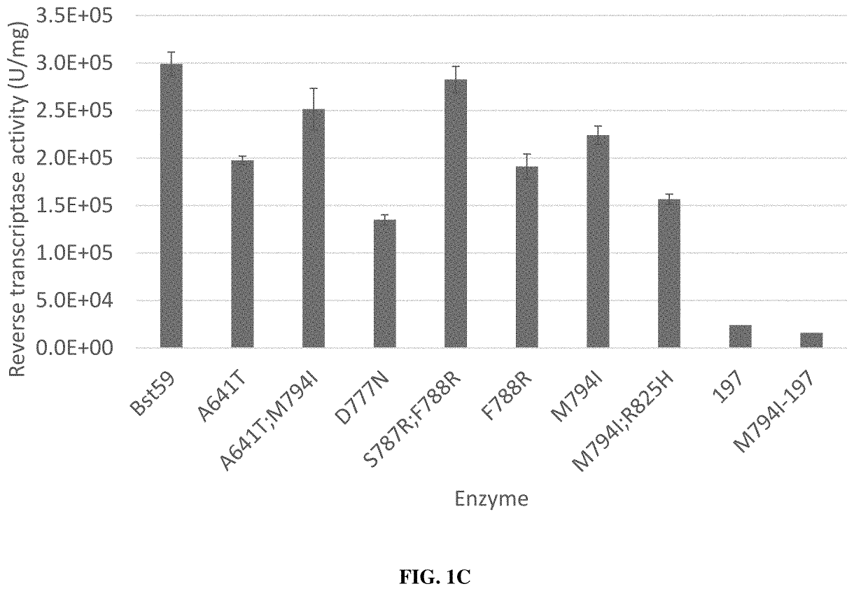

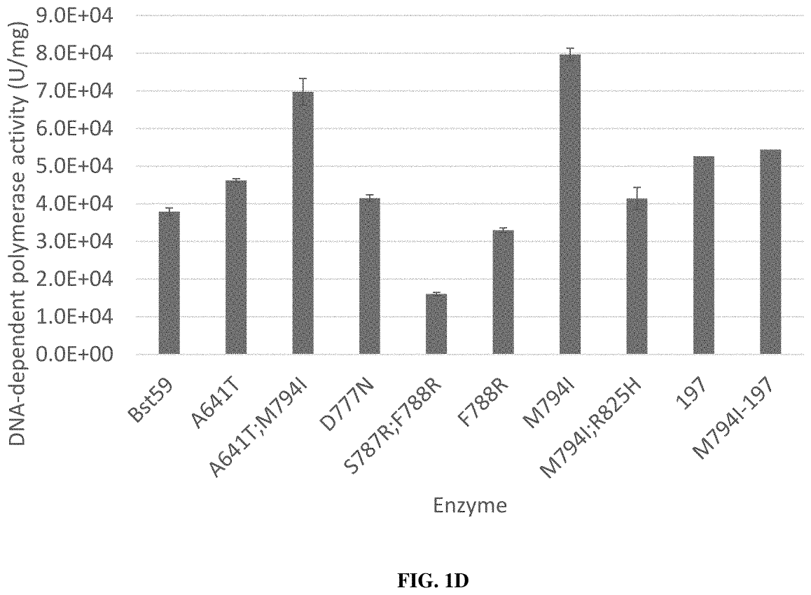

As will be understood, Bst polymerases are typically used as DNA polymerases; that is, as enzymes which catalyze the synthesis of deoxyribonucleic acid (DNA). However, the present inventors surprisingly observed that certain Bst polymerases possess reverse transcriptase activity, in addition to possessing DNA-dependent DNA polymerase activity (see FIGS. 1A-1D). Reverse transcriptases are enzymes that transcribe RNA to complementary DNA (cDNA) by polymerizing deoxyribonucleotide triphosphates (dNTPs), a process termed reverse transcription, and are used in the amplification of ribonucleic acid (RNA) targets. The Bst polymerase variants described herein can thus act as DNA polymerases and/or as reverse transcriptases, depending on the identity of the nucleic acid target being amplified.

Accordingly, in some embodiments, a target nucleic acid comprises a DNA sequence. In some embodiments, the target DNA is DNA from Aeromonas hydrophila. In some embodiments a target nucleic acid comprises a ribonucleic acid (RNA) sequence. In some embodiments, the target RNA is RNA from MS2, SARS-CoV-2, or human ribonuclease P (RP). In some embodiments, the target RNA is RNA from an influenza virus or a virus associated with a sexually-transmitted infection (STI), such as, for example, herpesvirus, hepatitis B, human papilloma virus (HPV), and human immunodeficiency virus (HIV). In some embodiments, the influenza virus disease is Influenza A. In some embodiments, the Influenza A virus is of the subtype H1N1, H2N2, H3N2, H5N1, H7N7, H1N2, H9N2, H7N2, H7N3, or H10N7. In some embodiments, the influenza virus disease is Influenza B. In some embodiments, the Influenza B virus is of the lineage Victoria or Yamagata. In some embodiments, the influenza virus disease is Influenza C. In some embodiments, a target nucleic acid sequence comprises a nucleic acid sequence from Neisseria gonorrhoeae and/or Chlamydia trachomatis.

In some embodiments, one or more mutations as described herein result in the Bst59 polymerase variant having increased reverse transcriptase activity for a given concentration of a target nucleic acid, relative to a control polymerase. In some embodiments, the control polymerase is selected from the group consisting of: the wild-type Bst nucleic acid polymerase having an amino acid sequence as shown in SEQ ID NO: 1, a nucleic acid polymerase variant having an amino acid sequence as shown in SEQ ID NO: 4, Bst 2.0, Bst 3.0, and Geobacillus species SSD polymerase large fragment (GspSSD 2.0 LF). In some embodiments, the Geobacillus species SSD polymerase large fragment (GspSSD 2.0 LF) is comprised within a buffer, such as, for example, ISO-004nd.

Though the Bst polymerase variants of the disclosure exhibit both DNA-dependent DNA polymerase activity and reverse transcriptase activity, it may nonetheless be desirable in some instances to add one or more additional enzymes having reverse transcriptase activity to the RT-LAMP reaction mixture to further increase the speed of the RT-LAMP reaction. However, the addition of a second enzyme having reverse transcriptase activity is not necessary to amplify RNA targets when using a Bst59 polymerase variant of the disclosure. In some embodiments, wherein the target nucleic acid is an RNA target, amplification of the target RNA occurs without the addition of a second enzyme having reverse transcriptase activity to the RT-LAMP reaction mixture. In some embodiments, wherein the target nucleic acid is an RNA target, amplification of the target RNA occurs with the addition of a second enzyme having reverse transcriptase activity.

Enzymes having reverse transcriptase activity (e.g., “reverse transcriptases”) are known in the art (see, e.g., Kati, et al. (1992), J. Biol. Chem., 267(36): 25988-97; Kotewicz, et al. (1985), Gene, 35(3): 249-58), and include, for example, RTx WarmStart (New England Biolabs, Cat. No. M0380; SuperScript IV (ThermoFisher Scientific, Cat. No. 18090010; and M-MLV (ThermoFisher Scientific, Cat. No. 28025013). Any suitable reverse transcriptase may be used as the second enzyme having reverse transcriptase activity. In some embodiments, the second enzyme having reverse transcriptase activity is a WarmStart® RTx Reverse Transcriptase (New England Biolabs, Cat. No. M0380). In some embodiments, the second enzyme having reverse transcriptase activity is a Human Immunodeficiency Virus (HIV) reverse transcriptase (Varigen Biosciences).

In some embodiments, the second enzyme having reverse transcriptase activity is a second DNA-dependent polymerase or a ribonuclease (RNase). A second DNA-dependent polymerase may comprise, for example, the wild-type B st nucleic acid polymerase having an amino acid sequence as shown in SEQ ID NO: 1, a nucleic acid polymerase variant having an amino acid sequence as shown in SEQ ID NO: 4, Bst 2.0, Bst 3.0, and Geobacillus species SSD polymerase large fragment (GspSSD 2.0 LF). In some embodiments, the Geobacillus species SSD polymerase large fragment (GspSSD 2.0 LF) is comprised within a buffer, such as, for example, ISO-004nd. An RNase generally refers to an enzyme that catalyzes the degradation of RNA. In some cases, an RNase may be used to digest RNA from an RNA-DNA hybrid.

In some embodiments, an amplification reaction mixture (e.g., a LAMP reaction mixture) comprises one or more LAMP primers and one or more additional reagents. In some embodiments, at least one (and, in some instances, each) of the one or more additional reagents is in liquid form (e.g., in solution). In some embodiments, at least one (and, in some instances, each) of the one or more additional reagents is in solid form. In certain embodiments, at least one (and, in some instances, each) of the one or more additional reagents is in solid form (e.g., lyophilized, dried, crystallized, air jetted).

In certain embodiments, the one or more additional reagents comprise one or more lysis reagents. A lysis reagent generally refers to a reagent that promotes cell lysis either alone or in combination with one or more reagents and/or conditions (e.g., heating). In some cases, the one or more lysis reagents comprise one or more enzymes. Non-limiting examples of suitable enzymes include lysozyme, lysostaphin, zymolase, cellulase, protease, and glycanase. In some embodiments, the one or more lysis reagents comprise one or more detergents. Non-limiting examples of suitable detergents include sodium dodecyl sulphate (SDS), Tween (e.g., Tween 20, Tween 80), 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), Triton X-100, and NP-40.

In some embodiments, the one or more lysis reagents comprise an RNase inhibitor (e.g., a murine RNase inhibitor). In certain embodiments, the RNase inhibitor concentration is at least 0.1 U/μL, at least 0.2 U/μL, at least 0.5 U/μL, at least 0.8 U/μL, at least 1.0 U/μL, at least 1.2 U/μL, at least 1.5 U/μL, at least 1.8 U/μL, or at least 2.0 U/μL. In certain embodiments, the RNase inhibitor concentration is in a range from 0.1 U/μL to 0.2 U/μL, 0.1 U/μL to 0.5 U/μL, 0.1 U/μL to 1.0 U/μL, 0.1 U/μL to 1.5 U/μL, 0.1 U/μL to 2.0 U/μL, 0.5 U/μL to 1.0 U/μL, 0.5 U/μL to 1.5 U/μL, 0.5 U/μL to 2.0 U/μL, or 1.0 U/μL to 2.0 U/μL. In some embodiments, the one or more lysis reagents comprise Tween (e.g., Tween 20, Tween 80).

In some embodiments, the one or more additional reagents comprise one or more reagents to reduce or eliminate potential carryover contamination from prior amplification rounds. In some embodiments, the one or more additional reagents comprise thermolabile uracil DNA glycosylase (UDG). In some cases, UDG may prevent carryover contamination from prior amplification rounds by degrading products that have already been amplified (i.e., amplicons) while leaving unamplified samples untouched and ready for amplification. In some embodiments, the concentration of UDG is at least 0.01 U/μL, at least 0.02 U/μL, at least 0.03 U/μL, at least 0.04 U/μL, or at least 0.05 U/μL. In certain embodiments, the concentration of UDG is in a range from 0.01 U/μL to 0.02 U/μL, 0.01 U/μL to 0.03 U/μL, 0.01 U/μL to 0.04 U/μL, or 0.01 U/μL to 0.05 U/μL.

In some embodiments, the one or more additional reagents comprise primers targeting nucleic acid sequences (e.g., Sars-CoV-2 sequences, influenza sequences, STD/STI sequences, etc.). Example LAMP primers for the detection of SARS-CoV-2, human RP gene, MS2, and Aeromonas hydrophila nucleic acid sequences are provided in Table 2, below. In some embodiments, a LAMP reaction mixture comprises one or more primer sequences as shown in Table 2. In some embodiments, a LAMP reaction mixture comprises one or more nucleic acid primers that is at least 80%, at least 85%, at least 90%, at least 95%, at least 96%, at least 97%, at least 98%, at least 99%, at least 99.5%, or 100% identical to a primer sequence provided in Table 2. However, it will be understood that primers targeting other nucleic acid sequences (e.g., influenza sequences, STD/STI sequences, etc.) can also be used.

TABLE 2
Example LAMP Primers

| | | |
|---|---|---|
| Primer | Sequence (5′ to 3′) | SEQ ID NO: |

Example Sars-CoV-2 LAMP Primers

| | | |
|---|---|---|
| F3_Set1 | CGGTGGACAAATTGTCAC | 123 |
| B3_Set1 | CTTCTCTGGATTTAACACACTT | 124 |
| Loop F_Set1 | TTACAAGCTTAAAGAATGTCTGAACACT | 125 |
| Loop B_Set1 | TTGAATTTAGGTGAAACATTTGTCACG | 126 |
| FIP1_Set1 | TCAGCACACAAAGCCAAAAATTTATCTGTGCAAAGGAAATTAAGGAG | 127 |
| BIP1_Set1 | TATTGGTGGAGCTAAACTTAAAGCCCTGTACAATCCCTTTGAGTG | 128 |
| FIP2_Set1 | TCAGCACACAAAGCCAAAAATTTATTTTTCTGTGCAAAGGAAATTAAGGAG | 129 |
| BIP2_Set1 | TATTGGTGGAGCTAAACTTAAAGCCTTTTCTGTACAATCCCTTTGAGTG | 130 |
| F3_Set2 | TGCTTCAGTCAGCTGATG | 131 |
| B3_Set2 | TTAAATTGTCATCTTCGTCCTT | 132 |
| FIP_Set2 | TCAGTACTAGTGCCTGTGCCCACAATCGTTTTTAAACGGGT | 133 |
| BIP_Set2 | TCGTATACAGGGCTTTTGACATCTATCTTGGAAGCGACAACAA | 134 |
| Loop F_Set2 | CTGCACTTACACCGCAA | 135 |
| Loop B_Set2 | GTAGCTGGTTTTGCTAAATTCC | 136 |

Example human RP gene LAMP Primers

| | | |
|---|---|---|
| Curtis F3 | TTGATGAGCTGGAGCCA | 137 |
| Curtis B3 | CACCCTCAATGCAGAGTC | 138 |
| Curtis FL | ATGTGGATGGCTGAGTTGTT | 139 |
| Curtis BL | CATGCTGAGTACTGGACCTC | 140 |
| Curtis FIP | GTGTKACCCTGAAGACTCGGTTTTAGCCACTGACTCGGATC | 141 |
| Curtis BIP | CCTCCGTGATATGGCTCTTCGTTTTTTTCTTACATGGCTCTGGTC | 142 |

Example MS2 LAMP Primers

| | | |
|---|---|---|
| MS2 F3 | TGTCATGGGATCCGGATGTT | 143 |
| MS2 B3 | CAATAGAGCCGCTCTCAGAG | 144 |
| MS2 FL | CCAGAGAGGAGGTTGCCAA | 145 |
| MS2 BL | TGCAGGATGCAGCGCCTTA | 146 |
| MS2 FIP | GCCCAAACAACGACGATCGGTAAAACCAGCATCCGTAGCCT | 147 |
| MS2 BIP | GCACGTTCTCCAACGGTGCTGGTTGCTTGTTCAGCGAACT | 148 |

Example Aeromonas hydrophila LAMP Primers

| | | |
|---|---|---|
| AH F3 | GCTGCTGATAATTCGGCTGTT | 149 |
| AH B3 | CTCTTAATCGAATCTCCAGGATCT | 150 |
| AH FL | AGAGCGGGGTTTAAATATATTGAA | 151 |
| AH BL | GCTACTTTTTTCACTGATTCCTGTT | 152 |
| AH FIP | GCACTTTATGAACGCTTGAAAGAAATAAAGCTATTACTGCCCCCTCG | 153 |
| AH BIP | AACTTGATTGTTTTCATTCCTGACTTGAGCGGTATGCTGTCGC | 154 |

In some embodiments, the concentrations of the F3 primer and the B3 primer in an amplification reaction mixture (e.g., a LAMP amplification reaction mixture) are each at least 0.05 μM, at least 0.06 μM, at least 0.07 μM, at least 0.08 μM, at least 0.09 μM, at least 0.1 μM, at least 0.2 μM, at least 0.3 μM, at least 0.4 μM, at least 0.5 μM, at least 0.6 μM, at least 0.7 μM, or at least 0.8 μM. In some embodiments, the concentrations of the F3 primer (and the B3 primer in an amplification reaction mixture (e.g., a LAMP amplification reaction mixture) are each in a range from 0.05 μM to 0.1 μM, 0.05 μM to 0.2 μM, 0.05 μM to 0.3 μM, 0.1 μM to 0.2 μM, 0.1 μM to 0.3 μM, 0.1 μM to 0.4 μM, 0.1 μM to 0.8 μM, or 0.5 μM to 0.8 μM. In some embodiments, the concentrations of the F3 primer and the B3 primer in an amplification reaction mixture (e.g., a LAMP amplification reaction mixture) are each about 0.2 μM.

In some embodiments, the concentrations of the FIP primer and the BIP primer in an amplification reaction mixture (e.g., a LAMP amplification reaction mixture) are each at least 1 μM, at least 1.1 μM, at least 1.2 μM, at least 1.3 μM, at least 1.4 μM, at least 1.5 μM, at least 1.6 μM, at least 1.7 μM, at least 1.8 μM, at least 1.9 μM, at least 2 μM, or at least 2.1 μM. In some embodiments, the concentrations of the FIP primer and the BIP primer in an amplification reaction mixture (e.g., a LAMP amplification reaction mixture) are each in a range from 1 μM to 1.1 μM, 1 μM to 1.2 μM, 1 μM to 1.3 μM, 1 μM to 1.4 μM, 1 μM to 1.5 μM, 1 μM to 1.6 μM, 1 μM to 1.7 μM, 1 μM to 1.8 μM, 1 μM to 1.9 μM, 1 μM to 2 μM, 1 μM to 2.1 μM, 1.3 μM to 1.9 μM, 1.4 μM to 1.8 μM, or 1.5 μM to 1.7 μM. In some embodiments, the concentrations of the FIP primer and the BIP primer in an amplification reaction mixture (e.g., a LAMP amplification reaction mixture) are each about 1.6 μM.

In some embodiments, the concentrations of the LF primer and the LB primer in an amplification reaction mixture (e.g., a LAMP amplification reaction mixture) are each at least 0.1 μM, at least 0.2 μM, at least 0.3 μM, at least 0.4 μM, at least 0.5 μM, at least 0.6 μM, at least 0.7 μM, at least 0.8 μM, at least 0.9 μM, or at least 1.0 μM. In some embodiments, the concentrations of the LF primer and the LB primer in an amplification reaction mixture (e.g., a LAMP amplification reaction mixture) are each in a range from 0.1 μM to 0.2 μM, 0.1 μM to 0.5 μM, 0.1 μM to 0.8 μM, 0.1 μM to 1.0 μM, 0.2 μM to 0.5 μM, 0.2 μM to 0.8 μM, 0.2 μM to 1.0 μM, 0.3 μM to 0.5 μM, 0.3 μM to 0.8 μM, 0.3 μM to 1.0 μM, 0.4 μM to 0.8 μM, 0.4 μM to 1.0 μM, 0.5 μM to 0.8 μM, 0.5 μM to 1.0 μM, or 0.8 μM to 1.0 μM. In some embodiments, the concentrations of the LF primer and the LB primer in an amplification reaction mixture (e.g., a LAMP amplification reaction mixture) are each about 0.4 μM.

### Methods of Detection

Aspects of the disclosure relate to methods of detecting a target nucleic acid sequence using the Bst59 polymerase variants of the disclosure. In some embodiments, the method comprises: (i) obtaining a biological sample from a subject; (ii) performing a nucleic acid amplification reaction configured to amplify the target nucleic acid sequence using a Bst polymerase variant of the disclosure; and (iii) detecting the presence or absence of the target nucleic acid sequence. In some embodiments, the method further comprises a step of adding a second enzyme having reverse transcriptase activity to the nucleic acid amplification reaction. Such second enzymes having reverse transcriptase activity are described elsewhere herein.

In some embodiments, the target nucleic acid sequence is a DNA sequence or an RNA sequence. In some embodiments, nucleic acid amplification reaction comprises LAMP or RT-LAMP. In some embodiments, wherein the target nucleic acid sequence is a DNA sequence, the nucleic acid amplification reaction comprises LAMP. In some embodiments, wherein the target nucleic acid sequence is an RNA sequence, the nucleic acid amplification reaction comprises RT-LAMP.

In some embodiments, a subject is a vertebrate animal (e.g., a mammal or reptile). In some embodiments, a mammalian subject is a human, a non-human primate, a dog, a cat, a hamster, a mouse, a rat, a pig, a horse, a cow, a donkey or a rabbit. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, a subject is a human, non-human primate, or mouse subject.

In some embodiments, a method of detection comprises obtaining a biological sample from a human subject (e.g., step (i)). Examples of biological samples include bodily fluids (e.g., mucus, saliva, blood, serum, plasma, amniotic fluid, sputum, urine, cerebrospinal fluid, lymph, tear fluid, feces, gastric fluid, vaginal fluid, or semen), cell scrapings (e.g., a scraping from the mouth or interior cheek), exhaled breath particles, or tissue extracts. In some embodiments, the biological sample comprises a mucus, saliva, sputum, blood, urine, vaginal, semen, or cell scraping sample.

In some embodiments, the biological sample comprises a nasal secretion. In certain instances, for example, the biological sample is an anterior nares specimen. An anterior nares specimen may be collected from a subject by inserting a swab element of a sample-collecting component into one or both nostrils of the subject for a period of time. In some embodiments, the period of time is at least 5 seconds, at least 10 seconds, at least 20 seconds, or at least 30 seconds. In some embodiments, the period of time is 30 seconds or less, 20 seconds or less, 10 seconds or less, or 5 seconds or less. In some embodiments, the period of time is in a range from seconds to 10 seconds, 5 seconds to 20 seconds, 5 seconds to 30 seconds, 10 seconds to 20 seconds, or 10 seconds to 30 seconds.

In some embodiments, the biological sample comprises a cell scraping. In certain embodiments, the cell scraping is collected from the mouth or interior cheek. The cell scraping may be collected using a brush or scraping device formulated for this purpose. The biological sample may be self-collected by the subject or may be collected by another individual (e.g., a family member, a friend, a coworker, a health care professional) using a sample-collecting component, such as a nasal swab or other apparatus.

In some embodiments, the biological sample comprises an oral secretion (e.g., saliva). In certain cases, the volume of saliva in the biological sample is at least 1 mL, at least 1.5 mL, at least 2 mL, at least 2.5 mL, at least 3 mL, at least 3.5 mL, or at least 4 mL. In some embodiments, the volume of saliva in the biological sample is in a range from 1 mL to 2 mL, 1 mL to 3 mL, 1 mL to 4 mL, or 2 mL to 4 mL.

The biological sample, in some embodiments, is collected from a human subject who is suspected of having a disease, disorder, or infection. In some embodiments, the disease, disorder, or infection is detected using a diagnostic test. In some embodiments, the disease, disorder, or infection is a viral disease, disorder, or infection. In some embodiments, the disease, disorder, or infection is a coronavirus (e.g., COVID-19) and/or influenza (e.g., influenza type A or influenza type B). However, the disclosure is not so limited, and other indications are also envisioned. In some embodiments, the disease, disorder, or infection is a non-viral, bacterial, or fungal disease, disorder, or infection. In some embodiments, the disease, disorder, or infection is a sexually-transmitted disease (e.g., STD), disorder, or infection (e.g., STI). In some embodiments, the STD or STI is human immunodeficiency virus (HIV), human papilloma virus (HPV), chlamydia, gonorrhea, genital herpes, syphilis, bacterial vaginosis, chancroid, a cytomegalovirus infection, granuloma inguinale (donovanosis), lymphogranuloma venereum, molluscum contagiosum, or trichomoniasis.

As will be understood by the skilled artisan, certain types of biological samples, such as anterior nares samples (e.g., nasal secretions), blood, urine, etc., contain extraneous biological material in addition to the nucleic acid sequence(s) of interest. Such extraneous biological material is in some embodiments problematic because it may inhibit the amplification reaction by a number of mechanisms. In some embodiments, the biological sample is purified prior to performing an isothermal nucleic acid amplification reaction (e.g., step (ii)) and/or prior to detecting the presence or absence of the target nucleic acid sequence (e.g., step (iii)). Methods of purification can include, but are not limited to, organic extraction (e.g., phenol-chloroform extraction), Chelex extraction, and solid-phase extraction (e.g., silica spin-columns or beads), and serve to separate the extraneous biological material from the nucleic acid(s) of interest.

In some embodiments, the biological sample is not purified prior to performing an isothermal nucleic acid amplification reaction (e.g., step (ii)) and/or prior to detecting the presence or absence of the target nucleic acid sequence (e.g., step (iii)).

In some embodiments, amplified nucleic acid sequences (i.e., amplicons) may be detected using any suitable method. In some embodiments, a target nucleic acid is detected using a lateral flow assay (LFA) strip, a colorimetric assay, a CRISPR/Cas method of detection, or is directly detected using hybridization.

To facilitate detection of a target nucleic acid, in some embodiments, one or more LAMP primers are chemically modified. In some embodiments, such chemical modification comprises the conjugation of one or more LAMP primers to a detectable label. In certain embodiments, the detectable label is a fluorescent label. In some instances, the fluorescent label is associated with a quenching moiety that prevents the fluorescent label from signaling until the quenching moiety is removed. Conjugation of one or more LAMP primers to a detectable label may be desirable in certain embodiments to visualize readout results, for example on a lateral flow assay strip. Non-limiting examples of suitable labels include biotin, streptavidin, fluorescein isothiocyanate (FITC), fluorescein amidite (FAM), fluorescein, and digoxigenin (DIG). In some cases, labeling one or more LAMP primers may result in labeled amplicons, which may facilitate detection (e.g., via a lateral flow assay, as described elsewhere herein).

In some embodiments, one or more LAMP primers are conjugated to FAM. In some embodiments, one or more LAMP primers are conjugated to biotin. In some embodiments, one or more LAMP primers are conjugated to FAM, and one or more LAMP primers are conjugated to biotin. In such embodiments, successful on-target amplification involving two or more LAMP primers (e.g., 2, 3, 4, 5, or 6 LAMP primers) generates amplicons labeled with both FAM and biotin. In some embodiments, one or more LAMP primers are conjugated to DIG. In some embodiments, one or more LAMP primers are conjugated to DIG, and one or more LAMP primers are conjugated to biotin. In such embodiments, successful on-target amplification involving two or more LAMP primers (e.g., 2, 3, 4, 5, or 6 LAMP primers) generates amplicons labeled with both DIG and biotin. In certain embodiments, a LAMP primer is labeled with two or more labels.

In some embodiments, amplified nucleic acid sequences are detected using a lateral flow assay strip. In some embodiments, a fluidic sample is transported through the lateral flow assay strip via capillary action. In some embodiments, the fluidic sample may comprise labeled amplicons. Non-limiting examples of suitable labels include biotin, streptavidin, fluorescein isothiocyanate (FITC), fluorescein amidite (FAM), fluorescein, and digoxigenin (DIG). In some cases, as an amplicon-containing fluidic sample flows through the lateral flow assay, a labeled nanoparticle binds to a label of an amplicon, thereby forming a particle-amplicon conjugate. In some embodiments, a particle-amplicon conjugate may be captured by one or more capture reagents (e.g., immobilized antibodies), and an opaque marking may appear. The marking may have any suitable shape or pattern (e.g., one or more straight lines, curved lines, dots, squares, check marks, x marks).

A lateral flow assay strip may comprise one or more test lines (e.g., a test line configured to detect the presence or absence of a pathogenic nucleic acid sequence) and one or more control lines (e.g., a control line configured to detect the presence or absence of a target nucleic acid sequence). In some instances, each test line of the lateral flow assay strip is configured to detect a different target nucleic acid sequence (e.g., Sars-CoV-2 sequences, influenza sequences, STD/STI sequences, etc.). In some instances, two or more test lines of the lateral flow assay strip are configured to detect the same target nucleic acid sequence. In some embodiments, a test line comprises a capture reagent (e.g., an immobilized antibody) configured to detect a target nucleic acid sequence. In some embodiments, a particle-amplicon conjugate may be captured by one or more capture reagents (e.g., immobilized antibodies), and an opaque marking may appear, as described above.

In certain instances, a control line is a lateral flow control line. In some cases, the lateral flow control line becoming detectable indicates that a liquid was successfully transported through the lateral flow assay strip. In some embodiments, a control line is a human (or animal) nucleic acid sequence control line. In some embodiments, for example, the human (or animal) nucleic acid sequence control line is configured to detect a nucleic acid sequence (e.g., RP) that is generally present in all humans (or animals). In some cases, the human (or animal) nucleic acid sequence control line becoming detectable indicates that a human (or animal) sample was successfully collected, nucleic acid sequences from the sample were amplified, and the amplicons were transported through the lateral flow assay strip. In some embodiments, the lateral flow assay strip comprises two or more control lines. In some instances, for example, the lateral flow assay strip comprises a human (or animal) nucleic acid sequence control line and a lateral flow control line. The control line(s) may have any suitable shape or pattern (e.g., one or more straight lines, curved lines, dots, squares, check marks, x marks). In some embodiments, the lateral flow control line becoming detectable (e.g., an opaque marking appears) indicates that human RP nucleic acid sequences are present in the sample.

In some embodiments, amplified nucleic acid sequences are detected using a colorimetric assay. In certain embodiments, for example, a fluidic sample is exposed to a reagent that undergoes a color change when bound to a target nucleic acid sequence (e.g., human RP DNA or RNA), such as with an enzyme-linked immunoassay. In some embodiments, the assay further comprises a stop reagent, such as sulfonic acid. That is, when the fluidic sample is mixed with the reagents, the solution turns a specific color (e.g., red) if the target nucleic acid is present, and the sample is positive. If the solution turns a different color (e.g., green), the target nucleic acid sequence is not present, and the sample is negative. In some embodiments, the colorimetric assay may be a colorimetric LAMP assay; that is, the LAMP reagents may react in the presence or absence of a target nucleic acid sequence (e.g., from human RP) to turn one of two colors.

In some embodiments, amplified nucleic acid sequences are detected using a CRISPR/Cas method of detection. CRISPR generally refers to Clustered Regularly Interspaced Short Palindromic Repeats, and Cas generally refers to a particular family of proteins. In some embodiments, the CRISPR/Cas detection platform can be combined with an isothermal amplification method to create a single step reaction (Joung, et al., (2020), Point-of-care testing for COVID-19 using SHERLOCK® diagnostics). For example, the amplification and CRISPR detection may be performed using reagents having compatible chemistries (e.g., reagents that do not interact detrimentally with one another and are sufficiently active to perform amplification and detection). In some embodiments, CRISPR/Cas detection is combined with LAMP. CRISPR/Cas detection platforms are known in the art. Examples of such platforms include SHERLOCK® and DETECTR® (see, e.g., Kellner, et al. (2019), Nature Protocols, 14: 2986-3012; Broughton, et al. (2020), Nature Biotechnology; Joung, et al. (2020)).

In some embodiments of a CRISPR/Cas method of detection, a guide RNA (gRNA) designed to recognize a specific target nucleic acid sequence (e.g., a human RNaseP nucleic acid sequence) may be used to detect target nucleic acid sequences present in a sample. If the sample comprises the target nucleic acid sequence, the gRNA will bind the target nucleic acid sequence and activate a programmable nuclease (e.g., a Cas protein), which may then cleave a reporter molecule and release a detectable signal (e.g., a reporter molecule tagged with specific antibodies for the lateral flow test, a fluorophore, a dye, a polypeptide, or a substrate for a specific colorimetric dye). In some embodiments, the detectable moiety binds to a capture reagent (e.g., an antibody) on a lateral flow strip, as described herein.

In some embodiments, amplified nucleic acid sequences are directly detected using hybridization.

Aspects of the disclosure relate to kits for the detection of a target nucleic acid sequence, and methods of making the same. In some embodiments, such kits comprise a Bst59 polymerase variant of the disclosure. In some embodiments, such kits further comprise a second enzyme having reverse transcriptase activity as described elsewhere herein.

### Additional Applications

Bst59 polymerase variants described herein may be used in any application utilizing a strand-displacing polymerase. In some embodiments, a Bst polymerase variant described herein is used in a nucleic acid sequencing method. In certain embodiments, the nucleic acid sequencing method is a long-read sequencing method. In certain embodiments, the nucleic acid sequencing method is a short-read sequencing method. In some embodiments, the nucleic acid sequencing method is a next-generation sequencing method.

### EXAMPLES

### Example 1: Optimization, Design, and Manufacture of Bst Polymerase Variants of the Disclosure

Novel nucleic acid polymerases comprising one or more mutations relative to a wild-type Bacillus stearothermophilus (Bst) nucleic acid polymerase were designed. As described elsewhere herein, the wild-type Bst sequence used is that of Geobacillus sp. WCH70 (Genbank Accession No. NC_012793) (SEQ ID NO: 1) (termed “Bst59” by the present inventors). Further information regarding Geobacillus sp. WCH70 (Bst59) can be found in Brumm, et al. (2016), Complete genome sequences of Geobacillus sp. WCH70, a thermophilic strain isolated from wood compost, Stand Genom Sci 11:33.

The novel Bst polymerases described herein have both DNA polymerase and reverse transcriptase (RT) capabilities, and can amplify both DNA and RNA targets without the addition of a second enzyme having RT activity. In some embodiments, an additional enzyme having RT activity is nonetheless added to the amplification reaction mixture, but doing so is not necessary to amplify RNA targets using a Bst polymerase variant of the disclosure.

### Optimization of Wild-Type Bst Sequence

The wild-type Bst59 nucleic acid polymerase (SEQ ID NO: 1) was optimized for LAMP by removal of the 5′ to 3′ exonuclease domain and for purification by addition of an N-terminal six-histidine (6×His) tag.

First, the 5′ to 3′ exonuclease domain of wild-type Bst59 (Geobacillus sp. WCH70 (Genbank Accession No. NC_012793); SEQ ID NO: 1), located at the N-terminus of the wild-type protein, was deleted. The 5′ to 3′ exonuclease domain which was deleted is shown as SEQ ID NO: 2. This deletion was made to avoid nucleolytic release of the fluorophores and quenchers conjugated to the LAMP primers.

Second, a six-histidine tag was added to the N-terminus of the 5′ to 3′ exonuclease domain-deficient Bst59. The six-histidine tag which was added is shown as SEQ ID NO: 3. This addition was made to enable traditional Ni-affinity column protein purification.

The resultant Bst59 sequence which comprises a deletion of a 5′ to 3′ exonuclease domain (SEQ ID NO: 2) and an addition of an N-terminal six-histidine tag (SEQ ID NO: 3) is shown as SEQ ID NO: 4, and is referred to herein as “optimized wild-type Bst59.”

### Design of Bst Polymerase Variants of the Disclosure

The Bst polymerase variants of the disclosure were designed to comprise one or more mutations relative to either the wild-type Bst59 sequence (Geobacillus sp. WCH70 (Genbank Accession No. NC_012793) shown in SEQ ID NO: 1 or to the optimized wild-type Bst59 sequence shown in SEQ ID NO: 4. The mutations are shown in Table 3. As described elsewhere herein, any of the mutations shown in Table 3 may be made alone or in combination. However, specific combinations of mutations which have demonstrated high reverse transcriptase activity and/or DNA-dependent DNA polymerase activity (see Example 3) are noted in Table 1 as SEQ ID NOs: 33-61.

For clarity, position numbering is shown in Table 3 relative to both SEQ ID NO: 1 (wild-type Bst59) and SEQ ID NO: 4 (optimized wild-type Bst59). However, position numbering relative to SEQ ID NO: 1 will be used throughout the application. As will be understood, the amino acid mutation(s) made are the same in either sequence; only the position numbers differ (due to the deletion of the 5′ to 3′ exonuclease domain from and addition of the N-terminal six-histidine tag to SEQ ID NO: 1 to produce SEQ ID NO: 4).

TABLE 3
Bst mutations

| Bst mutation(s) | Bst mutation(s) |
|---|---|
| (using position numbering | (using position numbering |
| of SEQ ID NO: 1) | of SEQ ID NO: 4) |
| N529K | N245K |
| K584Y | K300Y |
| N602A | N318A |
| N602L | N318L |
| I630G | I346G |
| A641T | A357T |
| I659K | I375K |
| V663I | V379I |
| L664M | L380M |
| I683V | I399V |
| T685K | T401K |
| I691V | I407V |
| M703L | M419L |
| R705V | R421V |
| Q706I | Q4221 |
| F712L | F428L |
| F712Y | F428Y |
| V715M | V431M |
| D720A | D436A |
| F745Y | F461Y |
| D777N | D493N |
| D777Q | D493Q |
| S787R | S503R |
| F788H | F504H |
| F788R | F504R |
| M794I | M510I |
| A802G | A518G |
| R825H | R541H |
| D832E | D548E |
| S787R; F788R | S503R; F504R |
| M794I; R825H | M510I; R541H |
| D777Q; M794I | D493Q; M510I |
| N529K; M794I | N245K; M510I |
| K584Y; M794I | K300Y; M510I |
| N602A; D832E | N318A; D548E |
| N602L; D832E | N318L; D548E |
| I630G; M794I | I346G; M510I |
| A641T; M794I | A357T; M510I |
| I659K; M794I | I375K; M510I |
| R705V; M794I | R421V; M510I |
| F712L; M794I | F428L; M510I |
| F712Y; M794I | F428Y; M510I |
| F788R; M794I | F504R; M510I |
| N529K; D777Q; M794I | N245K; D493Q; M510I |
| K584Y; D777Q; M794I | K300Y; D493Q; M510I |
| I630G; D777Q; M794I | I346G; D493Q; M510I |
| A641T; D777Q; M794I | A357T; D493Q; M510I |
| I659K; D777Q; M794I | I375K; D493Q; M510I |
| R705V; D777Q; M794I | R421V; D493Q; M510I |
| F712L; D777Q; M794I | F428L; D493Q; M510I |
| F712Y; D777Q; M794I | F428Y; D493Q; M510I |
| D777Q; S787R; F788R | D493Q; S503R; F504R |
| D777Q; F788R; M794I | D493Q; F504R; M510I |
| S787R; F788R; M794I | S503R; F504R; M510I |
| F712Y; D777Q; F788R; M794I | F428Y; D493Q; F504R; M510I |
| D777Q; S787R; F788R; M794I | D493Q; S503R; F504R; M510I |
| V663I; L664M; I683V; T685K; | V379I; L380M; I399V; T401K; |
| I691V; M703L; Q706I; V715M; | I407V; M419L; Q4221; V431M; |
| F745Y; A802G | F461Y; A518G |
| V663I; L664M; I683V; T685K; | V379I; L380M; I399V; T401K; |
| I691V; M703L; Q7061; V715M; | I407V; M419L; Q4221; V431M; |
| F745Y; M794I; A802G | F461Y; M510I; A518G |

The resultant Bst polymerase variants comprising one or more mutations—relative to either the wild-type Bst59 sequence (Geobacillus sp. WCH70 (Genbank Accession No. NC_012793)) shown in SEQ ID NO: 1 or to the optimized wild-type Bst59 sequence shown in SEQ ID NO: 4—are shown as SEQ ID NOs: 6-63.

### Manufacture and Purification of Bst Polymerase Variants of the Disclosure

The Bst polymerase variants of the disclosure were manufactured and purified as follows. The initial plasmid of SEQ ID NO: 4 was synthesized by ATUM (Newark, Calif.). All point mutations were introduced using Agilent's QuikChange II XL site-directed mutagenesis kit (Cat No. 200522) and the primers in Table 4 (SEQ ID NOs: 64-122), then subcloned into and expressed in Lucigen's E. coli 10G cell line (60108-1). Isolates of E. coli 10G-containing plasmids encoding Bst59 variants were stored in 50% glycerol at −80° C. before being grown in 100 mL Luria Broth (LB) with 30 μg/mL kanamycin and 0.4% glucose at 200 rpm and 30° C. overnight. The culture was then transferred to 2 L LB with 30 μg/mL kanamycin and 0.4% rhamnose for induction of protein expression for 24 hrs at 200 rpm and 30° C. The culture was centrifuged at 2200 rcf for 20 minutes and the pellet resuspended in 100 mL of 100 mM TrisHCl pH 8.0 with 250 mM NaCl and 6 mM imidazole. The cell suspension was sonicated using a Branson digital sonifier for 6 minutes using 15 s pulses at 50% power and 0° C. After sonication, the lysate was centrifuged for 30 minutes at 4° C. and 11000 rcf to clarify. The clarified lysate was applied to a 50 mL ThermoFisher HisPur Ni column equilibrated with 100 mM TrisHCl pH 8.0 with 250 mM NaCl and 6 mM imidazole. The column was washed with 250 mL of 100 mM TrisHCl pH 8.0 with 250 mM NaCl and 6 mM imidazole, and eluted with 250 mL of 100 mM TrisHCl pH 8.0 with 250 mM NaCl and 300 mM imidazole. Fractions containing the enzyme were pooled and diluted 1:1 with H₂O, and then applied to a 20 mL Q Sepharose column equilibrated with 50 mM TrisHCl pH 8.0 with 125 mM NaCl and 3 mM imidazole. The column was washed with 100 mL of 50 mM TrisHCl pH 8.0 with 125 mM NaCl and 3 mM imidazole, and the enzyme eluted with 100 mL of 100 mM TrisHCl pH 8.0 with 250 mM NaCl and 6 mM imidazole. The column was finally eluted with 100 mL of 100 mM TrisHCl pH 8.0 with 500 mM NaCl to remove nucleic acids and bound protein.

Large scale purifications were performed using frozen cell paste of the strains described herein, produced by Lytic Solutions at the 100-liter scale. An aliquot of this cell paste was resuspended in 200 mL of 100 mM TrisHCl pH 8.0 with 250 mM NaCl and 6 mM imidazole, sonicated for 9 minutes in 15 s pulses at 50% power and 0° C., then centrifuged at 11000 rcf for clarification. The clarified lysate was applied to a 60 mL HisPur Ni column equilibrated with 100 mM TrisHCl pH 8.0 with 250 mM NaCl and 6 mM imidazole. The column was washed with 300 mL of 100 mM TrisHCl pH 8.0 with 250 mM NaCl and 6 mM imidazole, and eluted with 300 mL of 100 mM TrisHCl pH 8.0 with 250 mM NaCl and 300 mM imidazole. Fractions containing the enzyme were pooled and diluted 1:1 with H₂O, and then applied to a 50 mL Q Sepharose column equilibrated with 50 mM TrisHCl pH 8.0 with 125 mM NaCl and 3 mM imidazole. The column was washed with 250 mL of 50 mM TrisHCl pH 8.0 with 125 mM NaCl and 3 mM imidazole, and the enzyme eluted with 250 mL of 100 mM TrisHCl pH 8.0 with 250 mM NaCl and 6 mM imidazole. The column was finally eluted with 250 mL of 100 mM TrisHCl pH 8.0 with 500 mM NaCl to remove nucleic acids and bound protein.

Fractions from Q Sepharose containing the enzyme were pooled and concentrated to 10-20 mL by ultrafiltration using a Pierce Protein Concentrator with a 10 kDa molecular weight cutoff (ThermoFisher Scientific, Cat. No. 88527). The concentrate was dialyzed against 50 volumes of Cas9 storage buffer (50 mM Tris-HCl pH 7.5 with 50 mM KCl, 1 mM DTT, 1 mM EDTA, and 50% glycerol) or Bst59 glycerol-free storage buffer (10 mM TrisHCl pH 7.5 with 50 mM KCl, 1 mM DTT, 0.1 mM EDTA, and 0.1% Tween-20).

TABLE 4
Primers used in cloning and sequencing of the exemplified
Bst59 polymerase variants of the disclosure

| Primer name | Primer sequence | SEQ ID NO |
|---|---|---|
| BstN529KDraIF | GAACTGGCGGGTCAAGAATTTAAAATCAATTCTCCGAAGCAACTG | 64 |
| BstN529KDraIR | CAGTTGCTTCGGAGAATTGATTTTAAATTCTTGACCCGCCAGTTC | 65 |
| BstK584YKpnIF | CCTGCACTATCGCCAGTTGGGGTACCTGCAGTCCACCTACATCGAAGG | 66 |
| BstK584YKpnIR | CCTTCGATGTAGGTGGACTGCAGGTACCCCAACTGGCGATAGTGCAGG | 67 |
| Bst59S587NSnaBIF | GTTGGGTAAGCTGCAGAATACGTACATCGAAGGCTTG | 68 |
| Bst59S587NSnaBIR | CAAGCCTTCGATGTACGTATTCTGCAGCTTACCCAAC | 69 |
| Bst59N602ABspHIF | GCTGAAAGTTGTTCATCATGACACGGCGAAAGTGCACACTATTTTC | 70 |
| Bst59N602ABspHIR | GAAAATAGTGTGCACTTTCGCCGTGTCATGATGAACAACTTTCAGC | 71 |
| Bst59N602LBspHIF | GCTGAAAGTTGTTCATCATGACACGCTGAAAGTGCACACTATTTTC | 72 |
| Bst59N602LBspHIR | GAAAATAGTGTGCACTTTCAGCGTGTCATGATGAACAACTTTCAGC | 73 |
| BstI630GEagIF | GCCGAATTTGCAGAACATCCCCGGCCGTCTGGAAGAGGGCCGTAAG | 74 |
| BstI630GEagIR | CTTACGGCCCTCTTCCAGACGGCCGGGGATGTTCTGCAAATTCGGC | 75 |
| BstA641TAclIF | GAGGGCCGTAAGATTCGCCAAACGTTCGTCCCGTCTGAGCCGG | 76 |
| BstA641TAclIR | CCGGCTCAGACGGGACGAACGTTTGGCGAATCTTACGGCCCTC | 77 |
| BstI659KSacIF | CTTTAGCGCCGACTATAGCCAGAAAGAGCTCCGTGTCCTGGCGCATATC | 78 |
| BstI659KSacIR | GATATGCGCCAGGACACGGAGCTCTTTCTGGCTATAGTCGGCGCTAAAG | 79 |
| BstR705VBsrGIF | GAAGTGACCCCGAACATGCGTGTACAGGCAAAAGCCGTGAATTTTG | 80 |
| BstR705VBsrGIR | CAAAATTCACGGCTTTTGCCTGTACACGCATGTTCGGGGTCACTTC | 81 |
| BstF712LHpaIF | CGCCGTCAGGCAAAAGCCGTTAACCTGGGTATCGTGTATGGTATCAGCG | 82 |
| BstF712LHpaIR | CGCTGATACCATACACGATACCCAGGTTAACGGCTTTTGCCTGACGGCG | 83 |
| BstF712YHpaIF | CGCCGTCAGGCAAAAGCCGTTAACTATGGTATCGTGTATGGTATCAGCG | 84 |
| BstF712YHpaIR | CGCTGATACCATACACGATACCATAGTTAACGGCTTTTGCCTGACGGCG | 85 |
| Bst59D720ANdeIF | GTGTATGGTATCAGCGCATATGGTCTGAGCCAAAAC | 86 |
| Bst59D720ANdeIR | GTTTTGGCTCAGACCATATGCGCTGATACCATACAC | 87 |
| Bst59D777NSpeIF | CAGACGTTATCTGCCGAATATCACTAGTCGCAACTTTAATCTG | 88 |
| Bst59D777NSpeIR | CAGATTAAAGTTGCGACTAGTGATATTCGGCAGATAACGTCTG | 89 |
| Bst59D777QSpeIF | CAGACGTTATCTGCCGCAGATCACTAGTCGCAACTTTAATCTG | 90 |
| Bst59D777QSpeIR | CAGATTAAAGTTGCGACTAGTGATCTGCGGCAGATAACGTCTG | 91 |
| Bst59SF787RRAscIF | CGCAACTTTAATCTGCGTCGGCGCGCCGAGCGTACGGCGATG | 92 |
| Bst59SF787RRAscIR | CATCGCCGTACGCTCGGCGCGCCGACGCAGATTAAAGTTGCG | 93 |
| Bst59F788HFspIF | CAACTTTAATCTGCGCAGCCATGCGGAGCGTACGGCG | 94 |
| Bst59F788HFspIR | CGCCGTACGCTCCGCATGGCTGCGCAGATTAAAGTTG | 95 |
| Bst59F788RPmlIF | CAACTTTAATCTGCGTTCACGTGCGGAGCGTACGGCG | 96 |
| Bst59F788RPmlIR | CGCCGTACGCTCCGCACGTGAACGCAGATTAAAGTTG | 97 |
| Bst59M794IAseIF | GGAGCGTACGGCGATTAATACGCCGATTCAG | 98 |
| Bst59M794IAseIR | CTGAATCGGCGTATTAATCGCCGTACGCTCC | 99 |
| Bst59M794IAseIF2 | GCCGAGCGTACGGCGATTAATACGCCGATTCAG | 100 |
| Bst59M794IAseIR2 | CTGAATCGGCGTATTAATCGCCGTACGCTCGGC | 101 |
| Bst59R825HNdeIF2 | GAAAAAAGAAAATATGAAAGCGCATATGCTGCTGCAAGTCCACGACG | 102 |
| Bst59R825HNdeIR2 | CGTCGTGGACTTGCAGCAGCATATGCGCTTTCATATTTTCTTTTTTC | 103 |
| Bst59D832EBspHIF2 | CGCGCATGCTGCTGCAAGTTCATGAAGAACTGATTTTAGAGGCGC | 104 |
| Bst59D832EBspHIR2 | GCGCCTCTAAAATCAGTTCTTCATGAACTTGCAGCAGCATGCGCG | 105 |
| Bst59-197VF1 | CGCGGACATCATCAAAAAAGCAATG | 106 |
| Bst59-197VR1 | CTCAATCTGGCTATAGTCGGCGC | 107 |
| Bst59-197IF1 | CCGACTATAGCCAGATTGAGCTTCGTATTATG | 108 |
| Bst59-197IR1 | CTTTTTTGATGATGTCCGCGCC | 109 |
| BstSeq1 | GGACGAATGCTTGGCGAATCTTAC | 110 |
| BstSeq2 | GAAAATTCCGGACGAGCCAGTG | 111 |
| BstSeq3 | GCCTTGTGGTAATTGCTCTCCAG | 112 |
| BstSeq4 | GACTACCACTTTGGTCCGACCTG | 113 |
| BstSeq5 | GTTCGTGCACACAGCCCAG | 114 |
| BstSeq6 | GAAAAAGCCGTTTCTGTAATGAAGGAG | 115 |
| BstSeq7 | CTGTTTCGCCTCTTGCACG | 116 |
| SL1 | CAGTCCAGTTACGCTGGAGTC | 117 |
| SR2 | GGTCAGGTATGATTTAAATGGTCAGT | 118 |
| T7 Terminator | GCTAGTTATTGCTCAGCGG | 119 |
| MS2F3b | TGTCATGGGATCCGGATGTTTTACAAACCA | 120 |
| MS2R | ACGGCGCACATTGGTCTCGGA | 121 |
| M13FT-41 | CGCCAGGGTTTTCCCAGTCACGAC | 122 |

### Example 2: Reverse Transcriptase and DNA-Dependent DNA Polymerase Activity of Bst Polymerase Variants of the Disclosure

### Reverse Transcriptase Activity

The reverse transcriptase (RT) activity of certain Bst polymerase variants of the disclosure was assessed (see FIGS. 1A and 1C).

RT activity was quantified using a modified SYBR Green I (ThermoFisher Scientific, Cat. No. S7563), Product-Enhanced Reverse Transcriptase RT-PCR assay (SG-PERT; Vermeire, et al. (2012), PLOS ONE, 7(12):e50859), using 12.5 ng of MS2 RNA in a 25 μL reaction and Q5 High-fidelity HotStart (New England Biolabs, Cat. No. M0493) as the DNA polymerase. This system was customized to use PicoGreen (ThermoFisher Scientific, Cat. No. P7581) instead of SYBR Green (PG-PERT), primers MS2 F3b/R, and Q5 HotStart High-fidelity DNA polymerase. The following 1× reaction recipe was used: 8.75 μL H₂O, 11.25 μL 2× Detect buffer (Detect, Inc.), 1 μL PicoGreen 1:16 diluted in H₂O, 0.5 μL 10 mM ea. dNTPs (New England Biolabs, Cat. No. N0447), 0.5 μL murine RNase inhibitor (New England Biolabs, Cat. No. M0314), 0.25 μL Q5 Hot Start High-Fidelity DNA polymerase, 0.125 μL 100 μM MS2 F3b/R (Integrated DNA Technologies), 0.125 μL 100 ng/μL untreated MS2 phage (Varigen Biosciences). The enzyme dilution range was 6.4 pg to 100 ng for wild-type optimized Bst59 and 0.5 mU to 8 U for Bst 3.0, each in a separate 25 μL reaction using 1× Detect buffer. The following thermocycler program was used: (1) 61.5° C., 20 minutes; (2) 98° C., 3 minutes; (3) 98° C., 5 seconds; (4) 56° C., 15 seconds; (5) 72° C., 15 seconds, plate read on SYBR/FAM channel; (6) go to step 3, 39×. Results were visualized with 1× PicoGreen dye. A standard curve of Bst 3.0 (New England Biolabs, Cat. No. M0374) was made using the Cqs of a triplicate, 7-step, 5-fold serial dilution from 8 U per 25 μL reaction. The RT activity of each enzyme was determined relative to known amounts of Bst 3.0, and the results of FIGS. 1A and 1C are shown in Units of Bst 3.0 activity (U)/mg.

As shown in FIG. 1A, both optimized wild-type Bst1 (SEQ ID NO: 5) and optimized wild-type Bst59 (SEQ ID NO: 4) possess reverse transcriptase (RT) activity, as assessed by PG-PERT assay; however, optimized wild-type Bst59 (SEQ ID NO: 4) exhibited greater RT activity than optimized wild-type Bst1 (SEQ ID NO: 5).

The RT activity of optimized wild-type Bst59 (SEQ ID NO: 4) was then compared to certain Bst59 variants of the disclosure: Bst59-A641T (SEQ ID NO: 11), Bst59-A641T;M794I (SEQ ID NO: 39), Bst59-D777N (SEQ ID NO: 25), Bst59-S787R;F788R (SEQ ID NO: 45), Bst59-F788R (SEQ ID NO: 29), Bst59-M794I (SEQ ID NO: 30), Bst59-M794I;R825H (alternately referred to herein as “M794IH” or “Bst59-M794IH”; SEQ ID NO: 33), Bst59-V663I;L664M;I683V;T685K;I691V;M703L;Q706I;V715M;F745Y;A802G (alternately referred to herein as “197” or “Bst59-197”; SEQ ID NO: 60), and Bst59-V663I;L664M;I683V;T685K; I691V;M703L;Q706I;V715M;F745Y;M794I;A802G (alternately referred to herein as “M794I-197” or “Bst59-M794I-197”; SEQ ID NO: 61). As shown in FIG. 1C, all of the exemplified Bst59 variants retained RT activity, with some variants maintaining levels which are comparable to those observed in optimized wild-type Bst59 (SEQ ID NO: 4). For example, the Bst59-A641T;M794I (SEQ ID NO: 39) and Bst59-S787R;F788R (SEQ ID NO: 45) variants exhibited RT activity comparable to those observed in optimized wild-type Bst59 (SEQ ID NO: 4).

### DNA-Dependent Polymerase Activity

The DNA-dependent polymerase activity of certain Bst polymerase variants of the disclosure was assessed (see FIGS. 1B and 1D).

The DNA-dependent DNA polymerase activity of each enzyme was determined relative to known amounts of Bst 2.0 WarmStart, and results are shown in Units of Bst 2.0 WarmStart activity (U)/mg. DNA-dependent polymerase activity was assessed using a single-stranded M13 primer extension assay with SYTO9 (ThermoFisher Scientific, Cat. No. 534854) at 61.5° C. for 10 minutes. The DNA-dependent DNA polymerase activity of each enzyme was determined relative to known amounts of Bst 2.0 WarmStart, and the results of FIGS. 1B and 1D are shown in Units of Bst 2.0 WarmStart activity (U)/mg.

As shown in FIG. 1B, both optimized wild-type Bst1 (SEQ ID NO: 5) and optimized wild-type Bst59 (SEQ ID NO: 4) possess DNA-dependent polymerase activity; however, optimized wild-type Bst59 (SEQ ID NO: 4) exhibited greater DNA-dependent polymerase activity than optimized wild-type Bst1 (SEQ ID NO: 5).

The DNA-dependent polymerase activity of optimized wild-type Bst59 (SEQ ID NO: 4) was then compared to certain Bst59 variants of the disclosure: Bst59-A641T (SEQ ID NO: 11), Bst59-A641T;M794I (SEQ ID NO: 39), Bst59-D777N (SEQ ID NO: 25), Bst59-S787R;F788R (SEQ ID NO: 45), Bst59-F788R (SEQ ID NO: 29), Bst59-M794I (SEQ ID NO: 30), Bst59-M794IH; SEQ ID NO: 33), Bst59-197 (SEQ ID NO: 60), and Bst59-M794I-197 (SEQ ID NO: 61). As shown in FIG. 1D, all of the exemplified Bst59 variants retained DNA-dependent polymerase activity, with some variants maintaining or exceeding the levels observed in optimized wild-type Bst59 (SEQ ID NO: 4). For example, the following Bst59 variants exhibited DNA-dependent polymerase activity exceeding the levels observed in optimized wild-type Bst59 (SEQ ID NO: 4): Bst59-A641T (SEQ ID NO: 11), Bst59-A641T;M794I (SEQ ID NO: 39), Bst59-D777N (SEQ ID NO: 25), Bst59-M794I (SEQ ID NO: 30), Bst59-M794IH; SEQ ID NO: 33), Bst59-197 (SEQ ID NO: 60), and Bst59-M794I-197 (SEQ ID NO: 61). Bst59-A641T;M794I (SEQ ID NO: 39) and Bst59-M794I (SEQ ID NO: 30) exhibited particularly improved levels of DNA-dependent polymerase activity.

Collectively, the results shown in FIGS. 1A-1D demonstrate (1) that optimized wild-type Bst1 (SEQ ID NO: 5) and optimized wild-type Bst59 (SEQ ID NO: 4) possess RT and DNA-dependent DNA polymerase activity, and that (2) certain exemplified Bst polymerase variants of the disclosure retain said RT activity and possess enhanced DNA-dependent DNA polymerase activity, relative to optimized wild-type Bst59 (SEQ ID NO: 4).

### Example 3: Bst Polymerase Variants of the Disclosure are Faster and More Sensitive than Existing State-of-the-Art DNA Polymerases

The speed at which certain novel Bst polymerases of the disclosure amplify target DNA and RNA sequences was assessed relative to the optimized Bst59 sequence shown in SEQ ID NO: 4, Bst 2.0 (New England Biolabs, Cat. No. M0537), Bst 3.0, and ISO-004nd (OptiGene, Cat. No. ISO-004nd). ISO-004nd Master Mix contains Geobacillus species SSD polymerase large fragment (GspSSD 2.0 LF), which according to the manufacturer has innate reverse transcriptase activity.

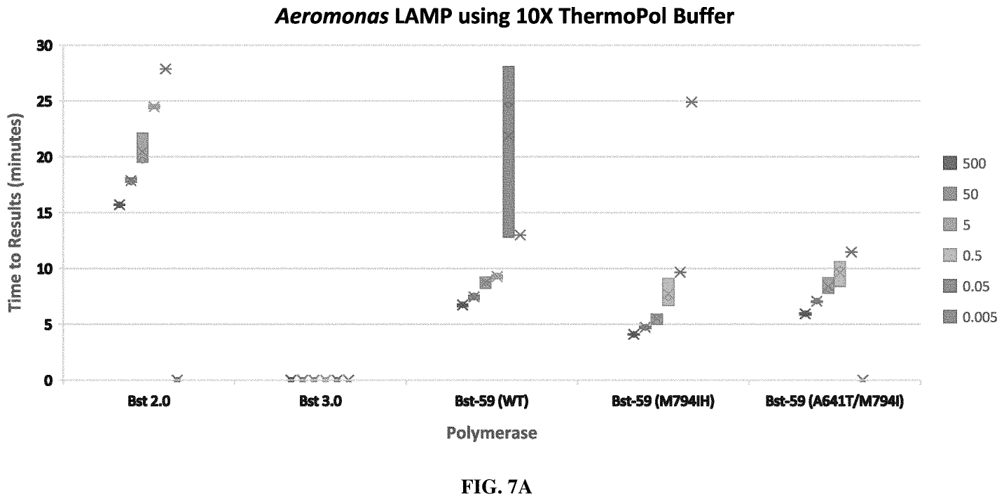

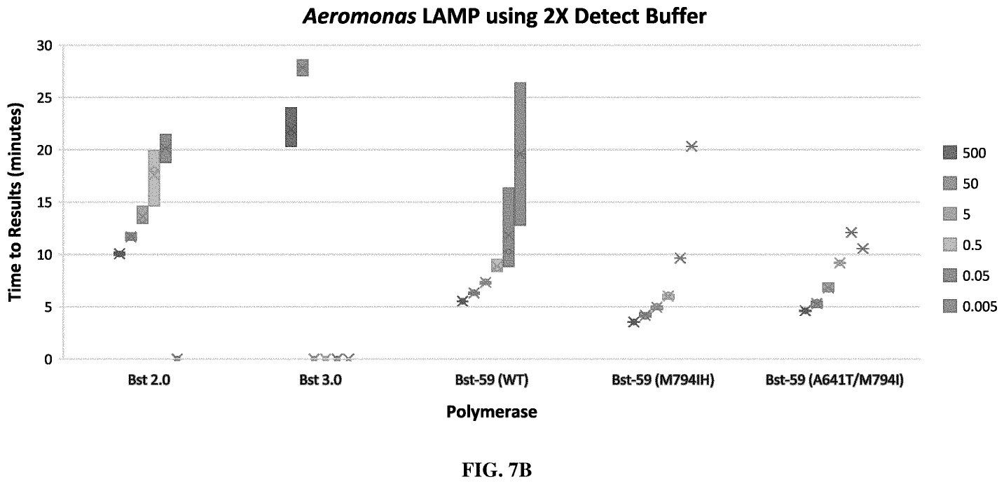

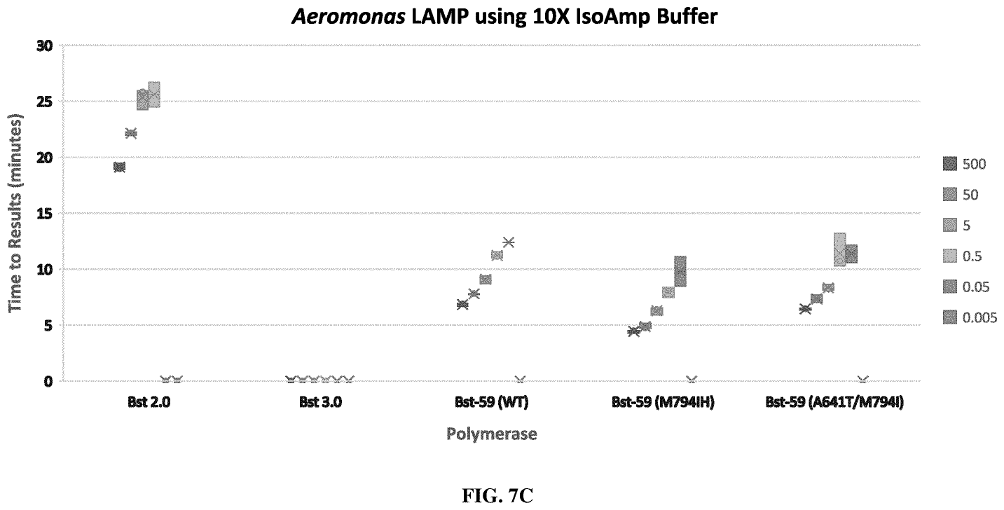

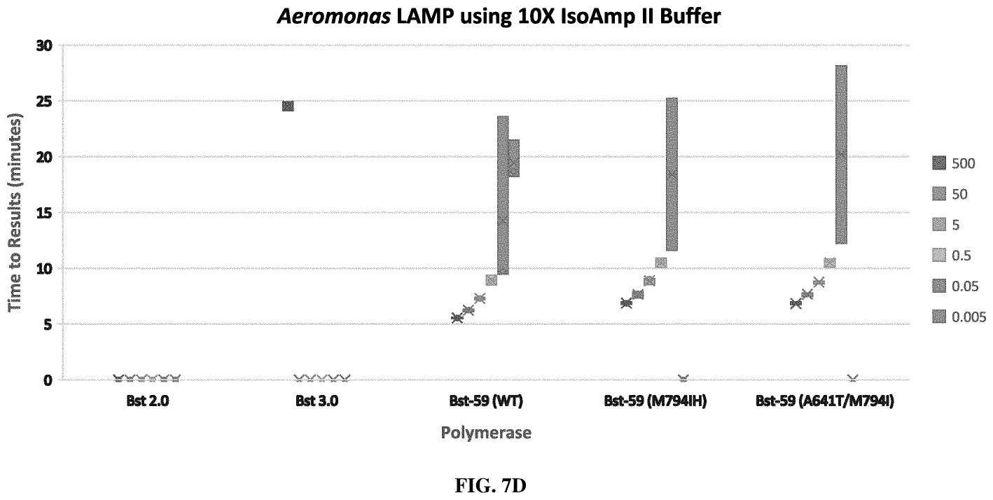

The novel Bst polymerases of the disclosure showed improved DNA polymerization activity in LAMP reactions, as compared to other existing, state-of-the-art Bst polymerases (see FIGS. 7A-7D).

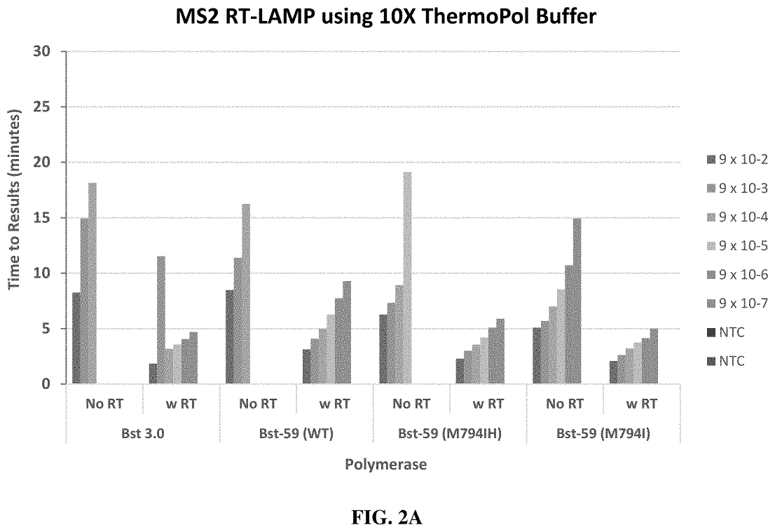

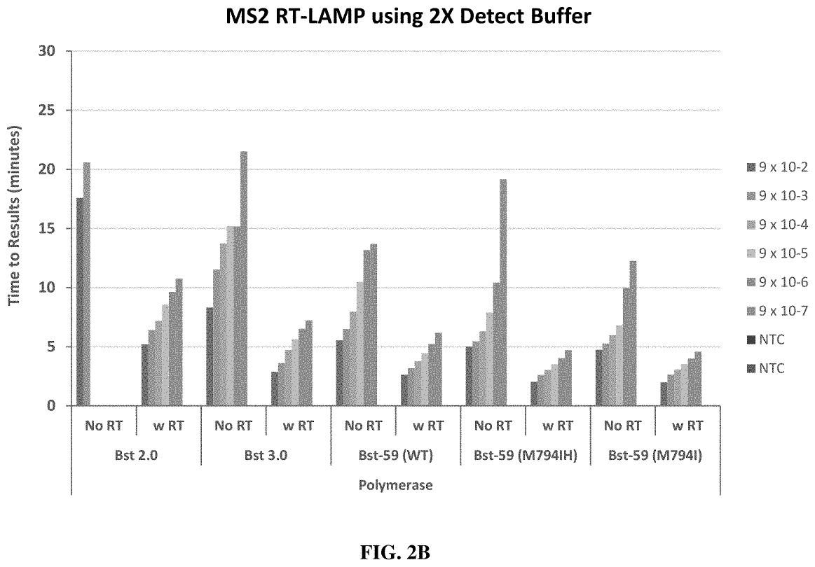

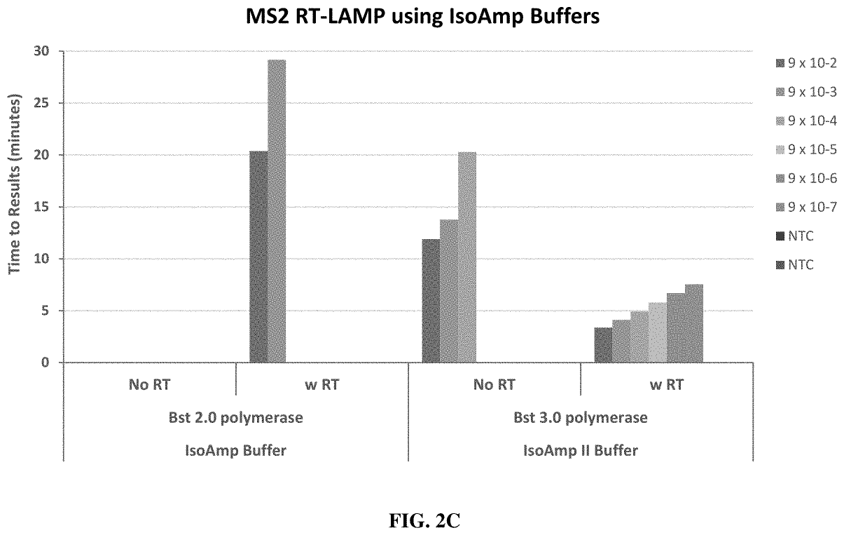

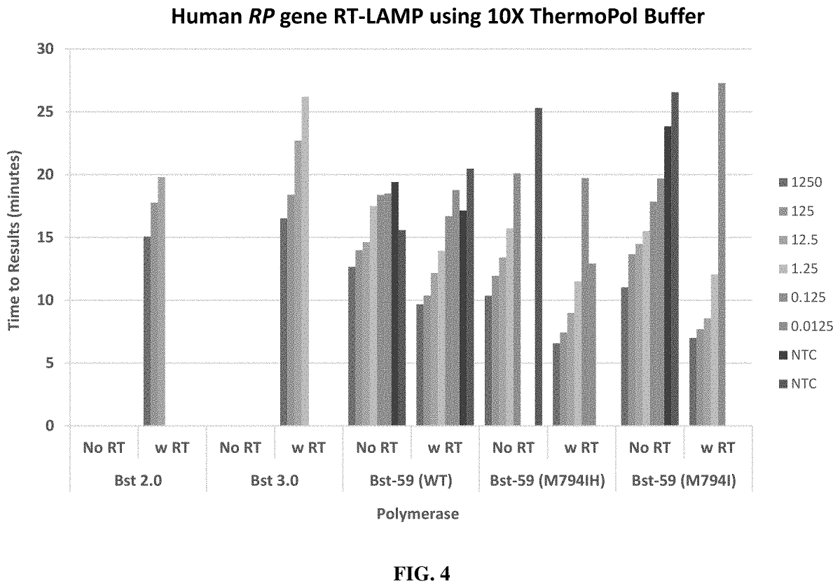

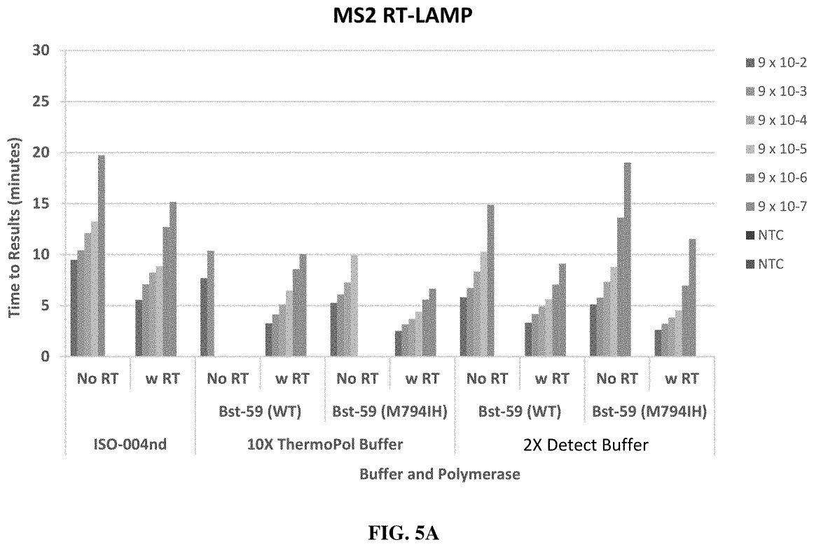

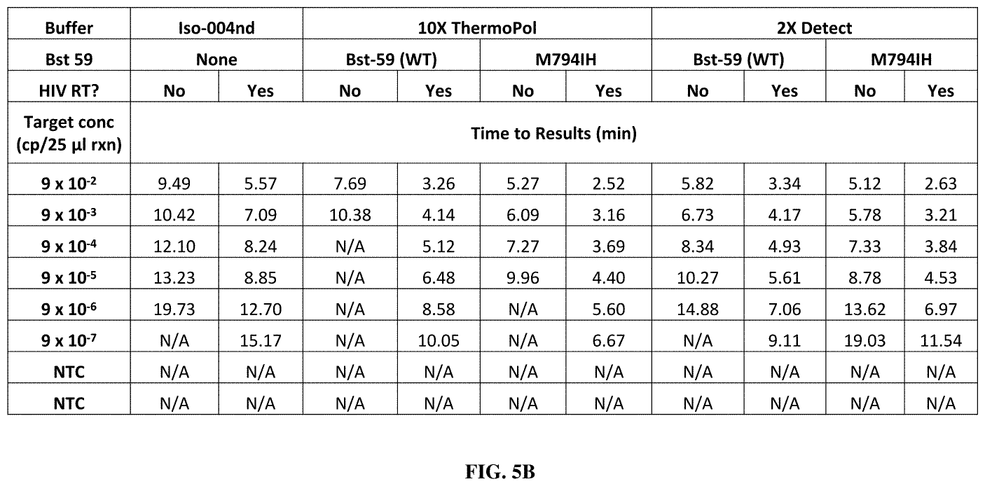

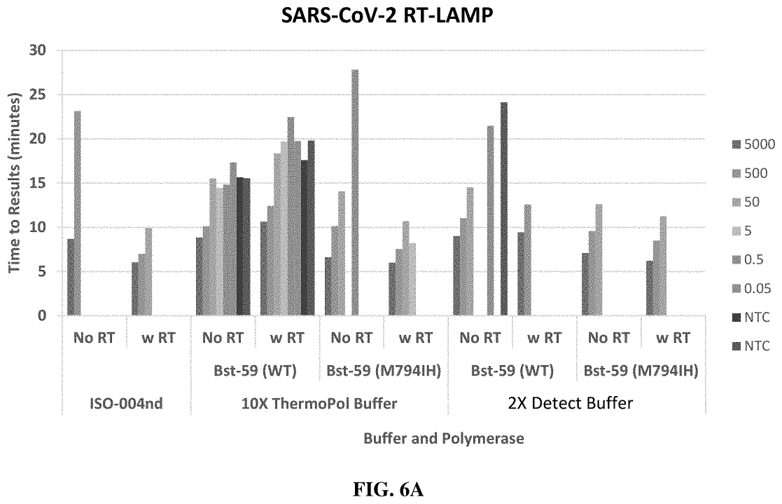

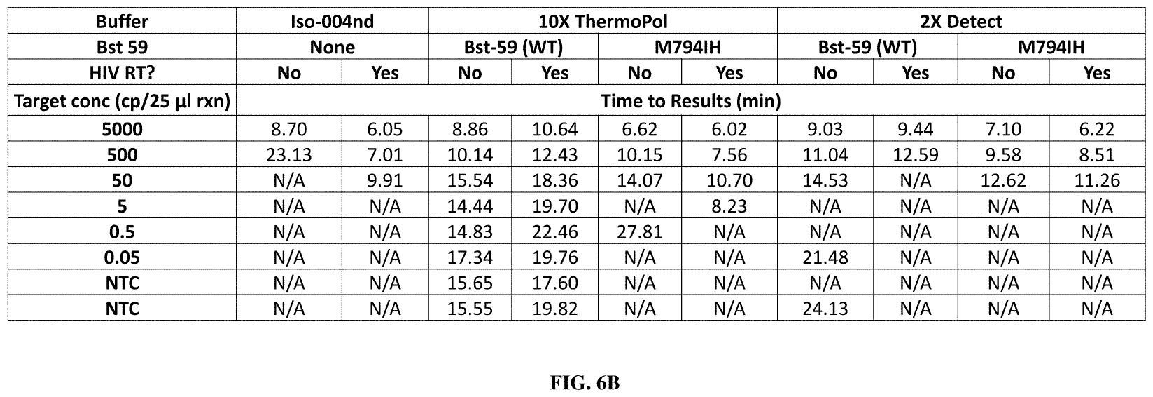

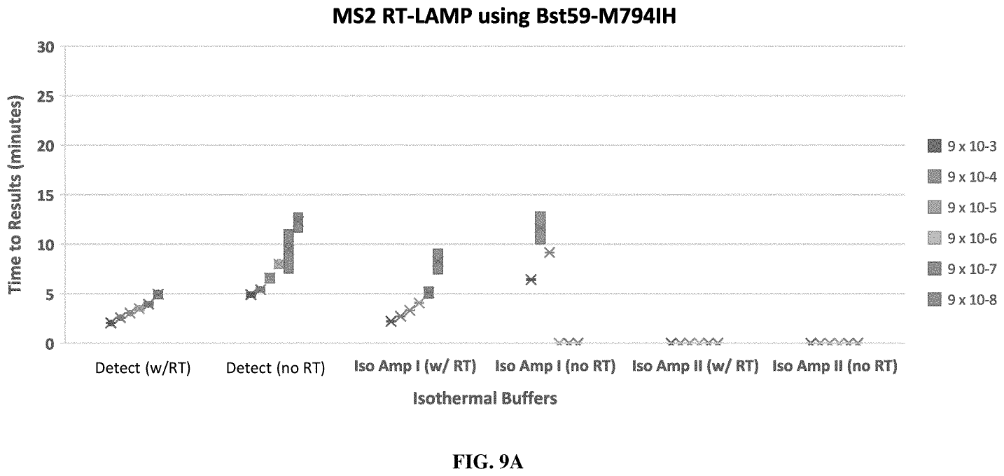

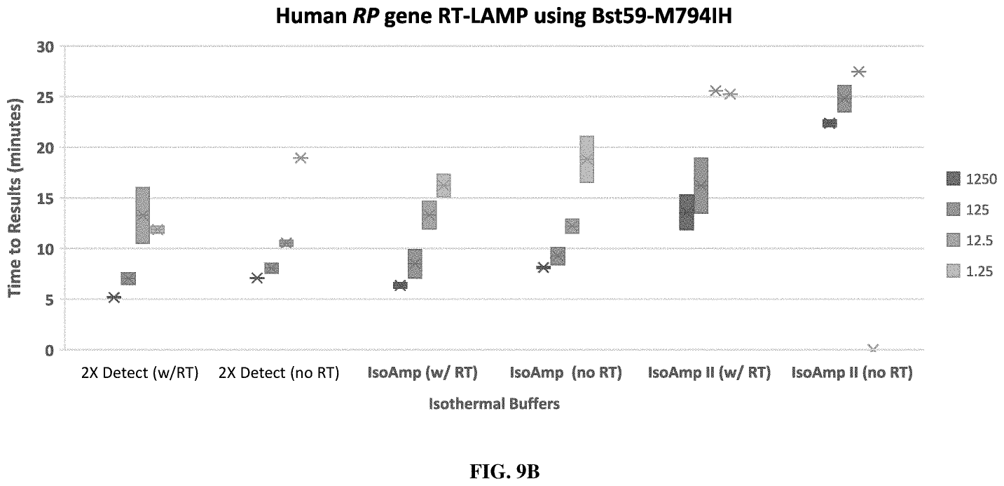

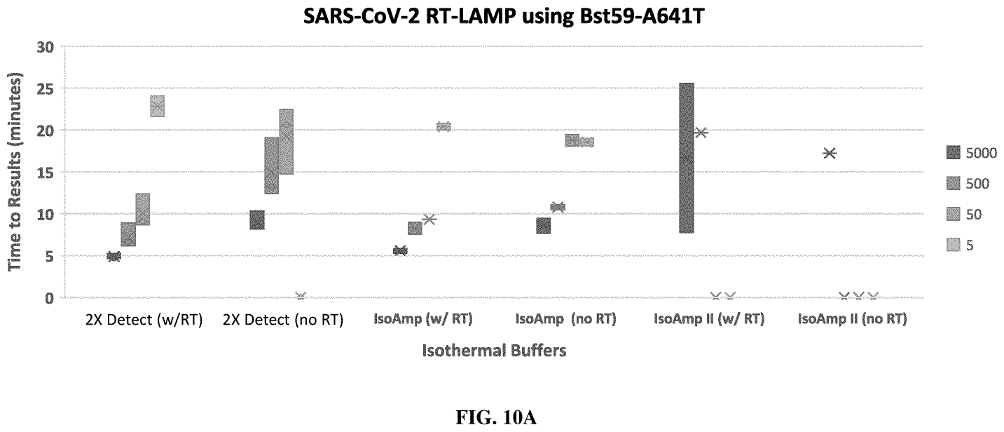

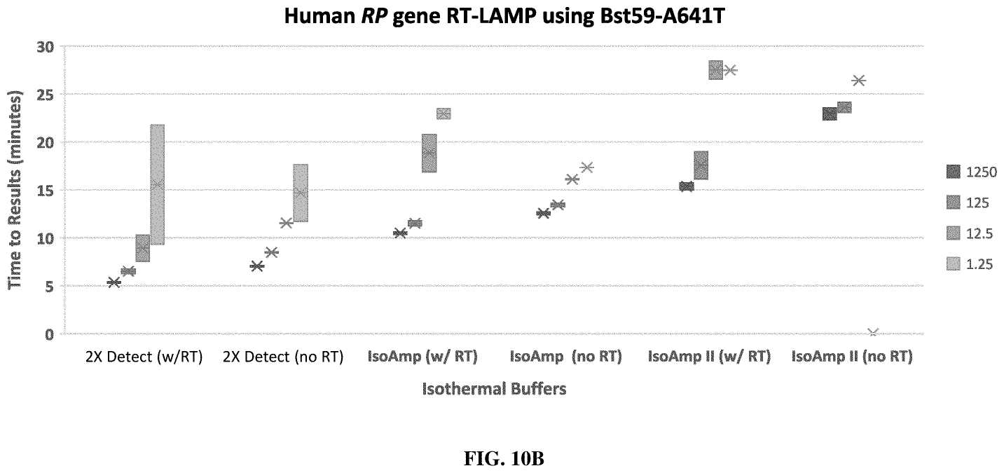

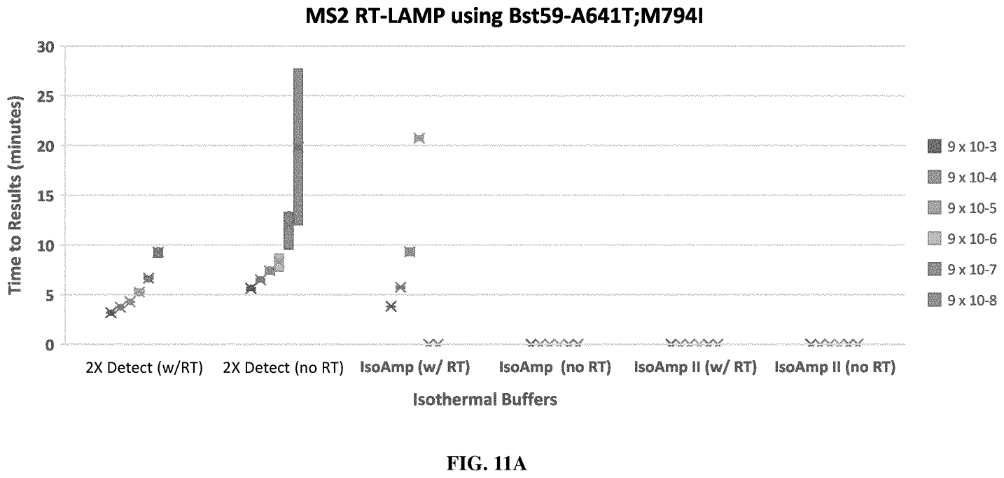

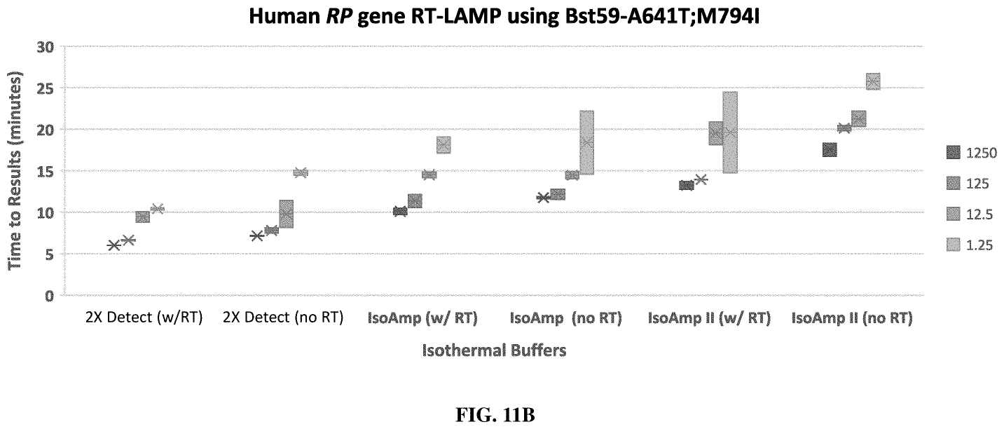

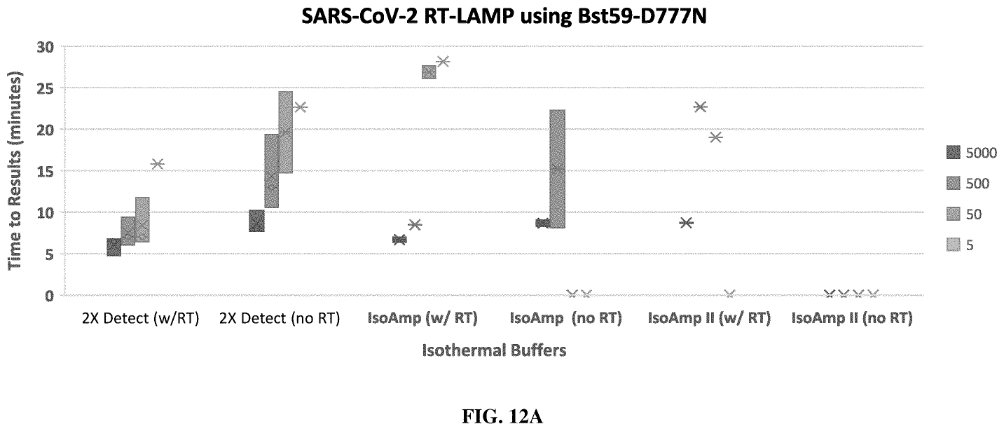

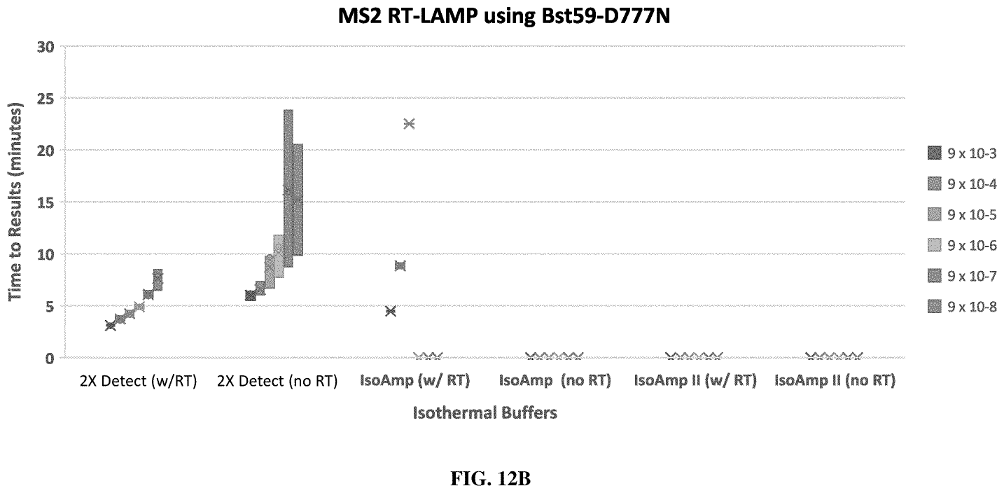

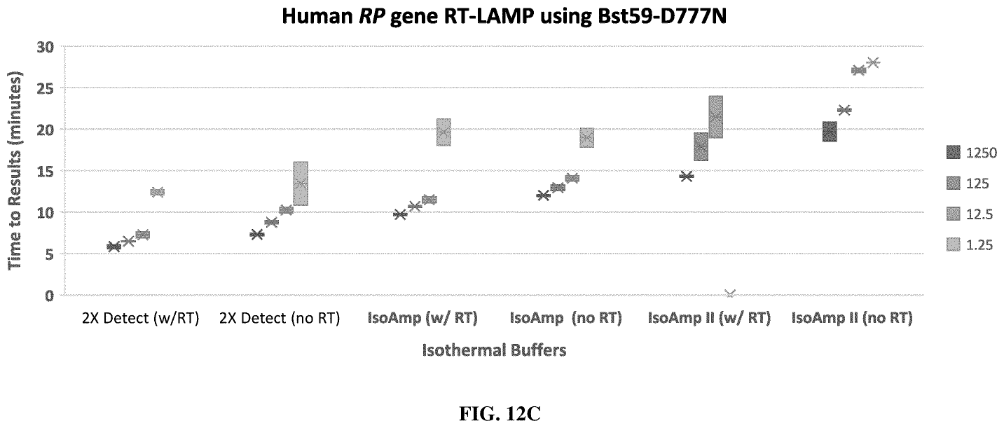

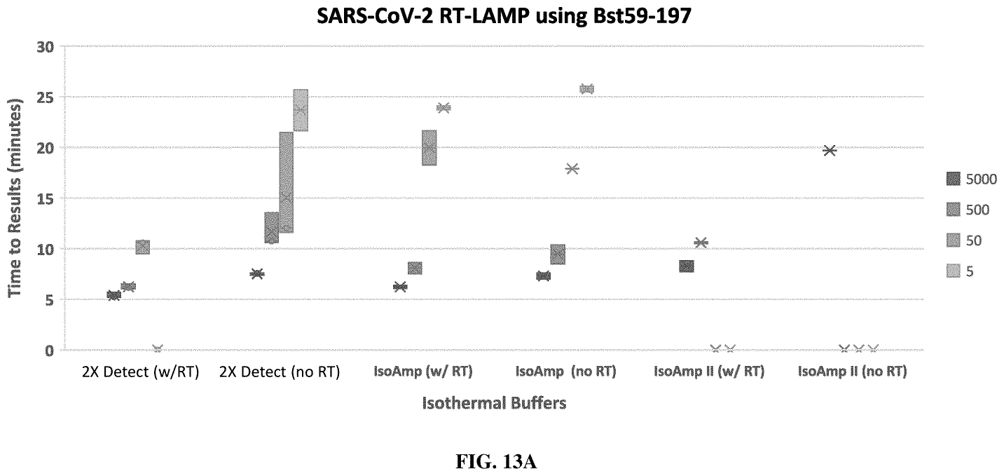

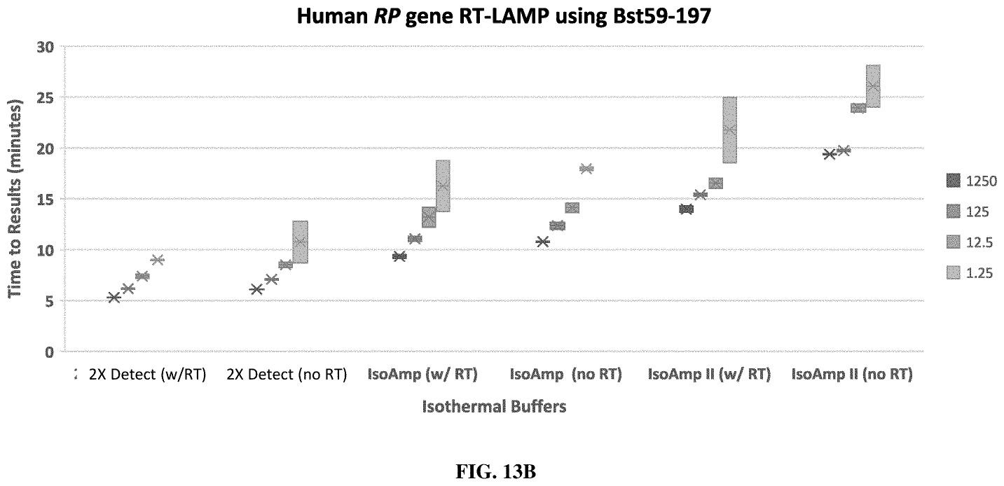

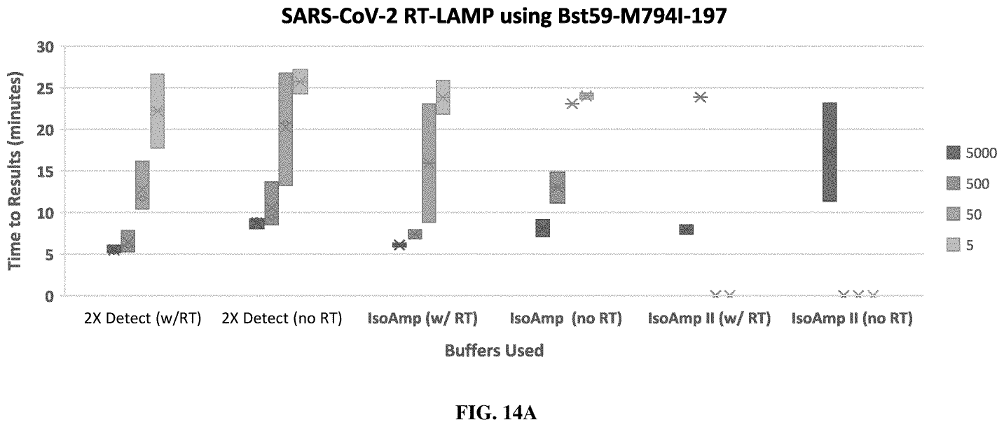

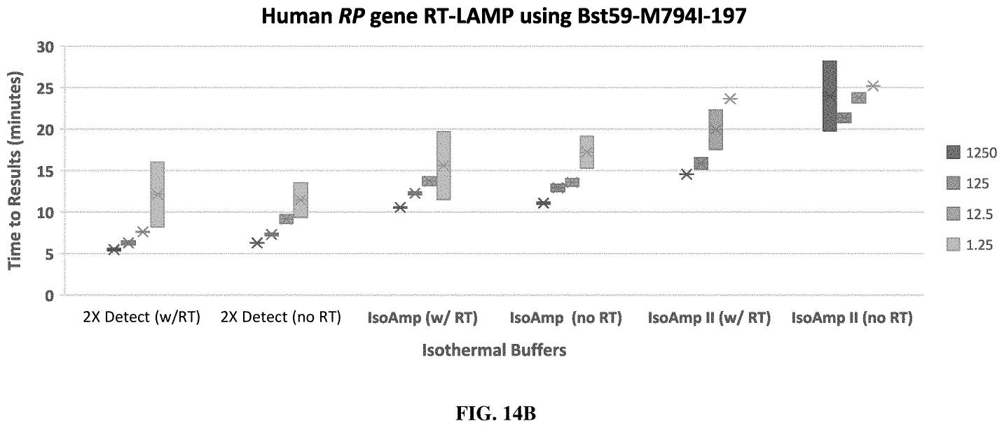

In addition, the novel Bst polymerases of the disclosure amplified several different RNA targets more efficiently in RT-LAMP reactions performed in several different buffers, as compared to other existing, state-of-the-art Bst polymerases. These results were maintained both with and without the addition of a second enzyme having RT activity (see FIGS. 2A-2C, 3A-3E, 4, 5A-5B, 6A-6B, 8A-8C, 9A-9B, 10A-10B, 11A-11B, 12A-12C, 13A-13B, and 14A-14B).

### Rapid Amplification of DNA Targets

As will be understood, Bst polymerases are DNA polymerases that are highly effective at amplifying DNA targets. The novel Bst polymerases of the disclosure maintain the ability to amplify DNA while also exhibiting RT activity, as described below and in Example 2. The retention of the ability to amplify DNA is desirable, for example, because the novel Bst polymerases of the disclosure can be used to amplify either DNA or RNA, depending on the identity of the target nucleic acid. Additionally, such retention may be useful in multiplexed approaches where DNA and RNA targets need be amplified in a single, multiplexed amplification reaction.

The DNA-dependent polymerase activity of certain Bst polymerase variants of the disclosure was assessed in LAMP reactions, and was compared to other existing, state-of-the-art Bst polymerases (see FIGS. 7A-7D).

DNA-dependent polymerase activity was assessed using a single-stranded M13 primer extension assay with SYTO9. The following 1× reaction recipe was used: 10.55 μL H₂O, 2 μL 10× Isothermal amplification buffer (New England Biolabs, Cat. No. B0537), 1.2 μL 100 mM MgSO4 (New England Biolabs, Cat. No. B1003), 3.5 μL 10 mM ea. dNTPs, 0.75 μL 100 μM SYTO9, 1 μL 1 μg/μL ssM13mp18 (Bayou Biolabs, Cat. No. P-107), 1 μL M13FT-41 (Integrated DNA Technologies; SEQ ID NO: 122). The following thermocycler program was used: (1) 61.5° C., 3 seconds, plate read on SYBR/FAM channel, (2) go to step 1, 39×. A standard curve of Bst 2.0 WarmStart (New England Biolabs, Cat. No. M0538) was made using the initial slopes of the amplification curves of a triplicate, 7-step, 2-fold serial dilution from 1 U per 25 μL reaction. Nucleic acids were amplified using a LAMP assay run at 68° C. for 30 minutes. DNA from Aeromonas was spiked into the LAMP reaction mixture at concentrations ranging from 500 to 0.005 copies/25 μL of reaction mixture. The activities of the Bst59 variants described herein were calculated from linear, in-range measurements derived from a similar dilution from 30 ng of polymerase/25 μL reaction.

As shown in FIGS. 7A through 7D, the Bst polymerase variants of the disclosure (Bst59-M794IH; (SEQ ID NO: 33) and Bst59-A641T;M794I (SEQ ID NO: 39)) not only maintained the ability to amplify DNA, but also showed improved DNA polymerization activity in LAMP reactions, as compared to other existing, state-of-the-art Bst polymerases (Bst 3.0 and optimized wild-type Bst59 (SEQ ID NO: 4)). These results were observed in LAMP reactions performed using various different buffers. The results shown in FIGS. 7A-7D thus demonstrate that the Bst polymerase variants of the disclosure (1) are capable of amplifying target DNA sequences at comparable or faster TTRs than both Bst 3.0 and optimized wild-type Bst59 and (2) that these results are consistent among various concentrations of target DNA and across various buffers.

### Rapid Amplification of RNA Targets

As described elsewhere herein, the novel Bst polymerases of the disclosure exhibit RT activity which is sufficient for amplification of RNA targets without the addition of a second enzyme having RT activity. This characteristic is desirable because, for typical amplification of RNA targets, a RT enzyme (e.g., AMV, MMLV, etc.) must be added to the amplification reaction mix, along with a DNA polymerase (e.g., Bst 2.0, Bst 3.0, Taq, etc.) to achieve sufficient speed. This addition of a second enzyme not only adds to the overall cost of each experiment, but also creates issues in terms of optimizing reaction conditions because of differences in the activity, optimum temperature, and inhibitor tolerance between the two enzymes.

However, it will be understood that a second enzyme having RT activity may, in some embodiments, be added to the amplification reaction mixture in order to further accelerate the speed of the RNA amplification reaction.

The RT activity of certain Bst polymerase variants of the disclosure was assessed in RT-LAMP reactions performed in several different buffers for several different RNA targets, and was compared to other existing, state-of-the-art Bst polymerases. The experiments were conducted in various buffers to ensure that the Bst polymerases of the disclosure could reliably amplify target RNA under a variety of experimental conditions. Results were obtained in LAMP reactions performed both with and without the addition of a second enzyme having RT activity (see FIGS. 2A-2C, 3A-3E, 4, 5A-5B, 6A-6B, 8A-8C, 9A-9B, 10A-10B, 11A-11B, 12A-12C, 13A-13B, and 14A-14B).

Bar height in each of FIGS. 2A-2C, 3A-3E, 4, 5A, 6A, 8A-8C, 9A-9B, 10A-10B, 11A-11B, 12A-12C, 13A-13B, and 14A-14B represents the time to results (TTR) for each polymerase. A short bar indicates that less time is needed before the RNA template is exponentially amplified and is evidence of fast amplification kinetics.

Certain exemplified Bst polymerase variants of the disclosure, including the variant Bst59-M794I (SEQ ID NO: 30), variant Bst-M794IH (SEQ ID NO: 33), variant Bst59-A641T (SEQ ID NO: 11), variant Bst59-A641T;M794I (SEQ ID NO: 39), variant Bst59-D777N (SEQ ID NO: 25), variant Bst59-197 (SEQ ID NO: 60), and variant Bst59-M794I-197 (SEQ ID NO: 61), were used to amplify RNA targets (MS2, SARS-CoV-2, and RP) of known concentrations both with and without the addition of a second enzyme having reverse transcriptase activity (see FIGS. 2A-2B, 3A-3B, 3D-3E, 4, 5A-5B, 6A-6B, 8A-8C, 9A-9B, 10A-10B, 11A-11B, 12A-12C, 13A-13B, and 14A-14B). Optimized wild-type Bst59 (SEQ ID NO: 4), Bst 2.0, Bst 3.0, and Iso-004nd were also tested as controls, both with and without the addition of a second enzyme having reverse transcriptase activity (see FIGS. 2A-2C, 3A-3E, 4, 5A-5B, and 6A-6B). ISO-004nd Master Mix contains Geobacillus species SSD polymerase large fragment (GspSSD 2.0 LF), which according to the manufacturer has innate reverse transcriptase activity. Loop-mediated isothermal amplification (LAMP) was used as the method of amplification.

Briefly, the method involved mixing RNA template with Master Mix containing optimized concentrations of MgSO4, dNTP mix, Bst polymerase, and intercalating dye (SYTO-82) for real-time monitoring of nucleic acid amplification. This was combined with target-specific primer sets. The reactions were performed in a real-time thermocycler (CFX-96, BioRad) for 30 minutes at a designated temperature (64° C. for SARS-CoV-2 and human RP gene; 72° C. for MS2). Reaction kinetics were monitored in real-time by measuring the increase in fluorescence associated with the accumulation of double-stranded DNA by LAMP.

As shown in FIGS. 2A-2B, 3A-3B, 3D-3E, 4, 5A-5B, and 6A-6B, Bst polymerase variants of the disclosure were both more sensitive and faster in amplifying the target RNA without addition of RT-polymerase, as measured by TTR, than any of optimized wild-type Bst59 (SEQ ID NO: 4), Bst 2.0, and Bst 3.0. These results were replicated in three different buffers and using three different target RNA templates.

For example, in 10× ThermoPol buffer (Varigen Biosciences) using MS2 RNA as the template, the limit of detection (LOD) was 9×10⁻⁷ μg/25 μL reaction in 15 minutes with Bst59-M794I, and was 9×10⁻⁵ μg/25 μL reaction in 19 minutes with Bst59-M794IH, without addition of a second enzyme having reverse transcriptase activity (FIG. 2A). The results observed for the Bst polymerase variants of the disclosure were much faster than Bst 3.0 (LOD: 9×10⁴ μg/25 μL reaction in 18 minutes) and optimized wild-type Bst59 (SEQ ID NO: 4) (LOD: 9×10⁴ μg/25 μL reaction in 16 minutes) (FIG. 2A).

In 2× Detect buffer using MS2 RNA as template, the observed LOD for Bst59-M794I was 9×10⁻⁷ μg/25 μL reaction in 12.3 minutes, and was 9×10⁻⁵ μg/25 μL reaction in 19.15 minutes for Bst59-M794IH, without addition of a second enzyme having reverse transcriptase activity (FIG. 2B). This is similar to LOD which was observed using optimized wild-type Bst59 (SEQ ID NO: 4) (LOD: 9×10⁻⁷ μg/25 μL reaction in 13.7 minutes), and was much faster than Bst 3.0 (LOD: 9×10⁻⁷ μg/25 μL reaction in 21.5 minutes) and Bst 2.0 (LOD: 9×10⁻³ μg/25 μL reaction in 20.5 minutes) (FIG. 2B). In IsoAmp II buffer using Bst 3.0, the observed LOD was 9×10⁴ μg/25 μL reaction in 20 minutes (FIG. 2C).

The addition of a second enzyme having reverse transcriptase activity to the reaction mix only improved the observed LOD and TTR, for all the tested enzymes (Bst 2.0, Bst 3.0, optimized wild-type Bst59, Bst59-M794I, and Bst59-M794IH) in both 10× ThermoPol buffer and 2× Detect buffer. Each of Bst59-M794I and Bst59-M794IH had faster TTRs than the other tested enzymes (Bst 2.0, Bst 3.0, optimized wild-type Bst59) (FIGS. 2A-2B).

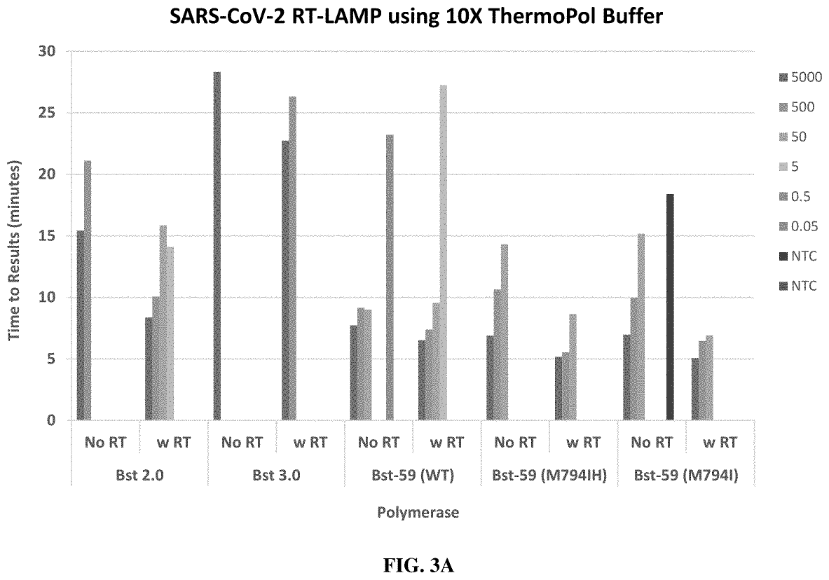

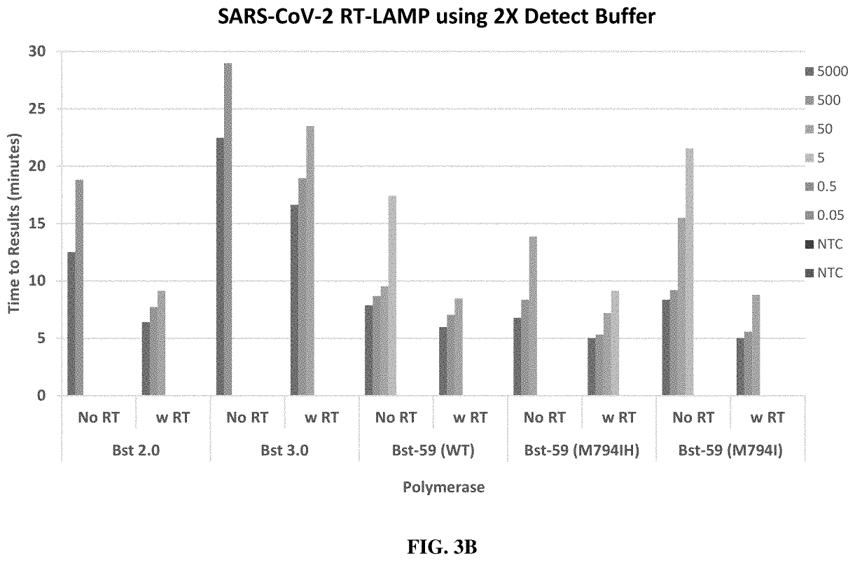

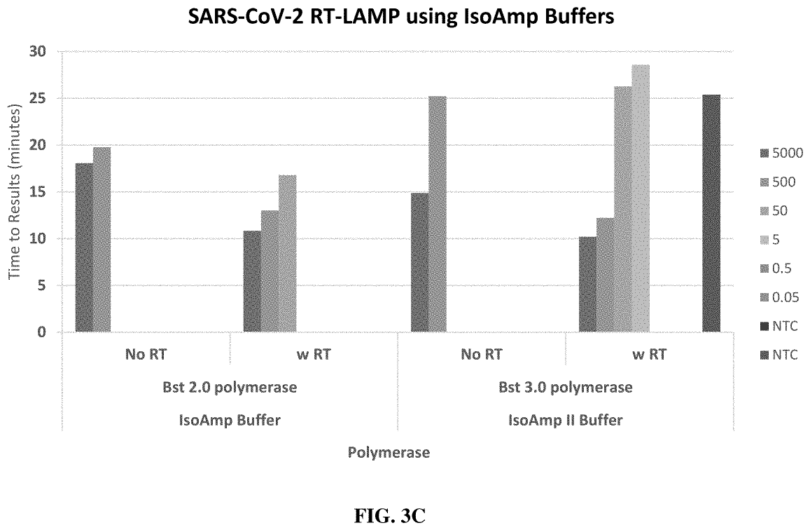

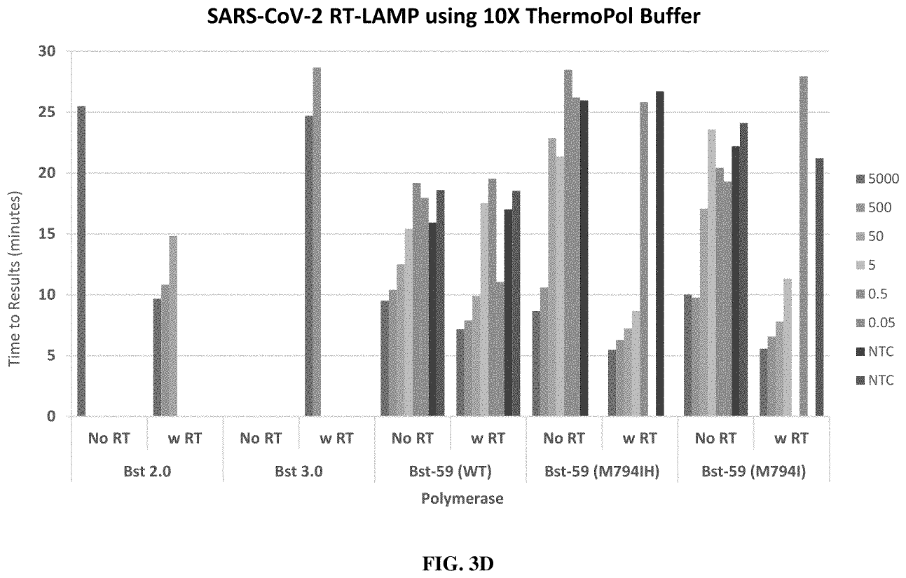

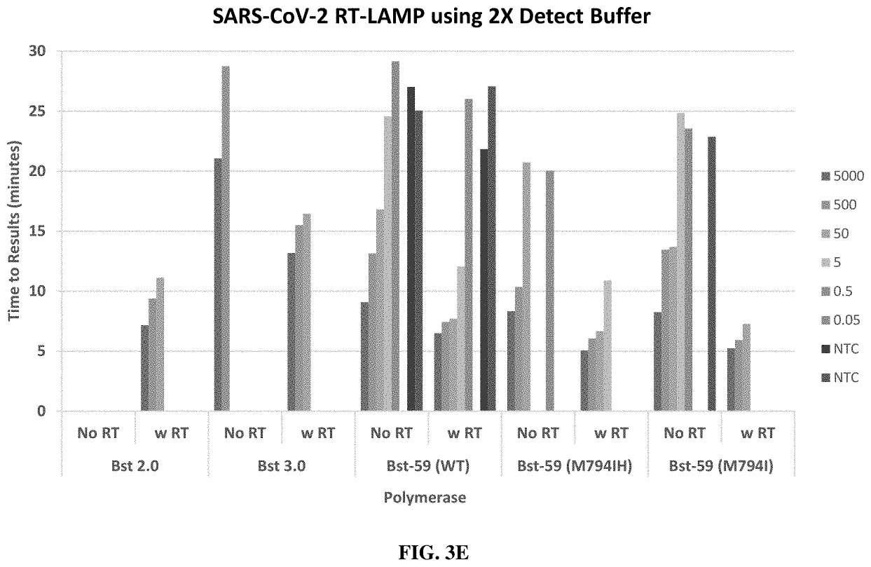

In 10× ThermoPol buffer using SARS-CoV-2 RNA as template, the observed LOD was 50 copies/25 μL reaction in 15 minutes for Bst59-M794I and Bst59-M794IH, without addition of a second enzyme having reverse transcriptase activity (FIG. 3A). This was much faster than Bst 3.0 (LOD: 500 copies/25 μL reaction in 26 minute) and Bst 2.0 (LOD: 500 copies/25 μL reaction in 21 minutes) (FIG. 3A).

In 2× Detect buffer using SARS-CoV-2 RNA as template, the observed LOD was 5 copies/25 μL reaction in 21 minutes for Bst59-M794I, and was 50 copies/25 μL reaction in 14 minutes for Bst59-M794IH, without addition of a second enzyme having reverse transcriptase activity (FIG. 3B). Using optimized wild-type Bst59 (SEQ ID NO: 4), the observed LOD was copies/25 μL reaction in 17 minutes. The TTRs observed using the Bst59 variants of the disclosure were much faster than Bst 3.0 (LOD: 500 copies/25 μL reaction in 28 minutes) and Bst 2.0 (LOD: 500 copies/25 μL reaction in 19.5 minutes) (FIG. 3B), even without using a second enzyme having reverse transcriptase activity. In IsoAmp II buffer using Bst 3.0, the observed LOD was 500 copies/25 μL reaction in 25 minutes, without addition of a second enzyme having reverse transcriptase activity (FIG. 2C).

The addition of a second enzyme having reverse transcriptase activity to the reaction mix only improved the observed LOD and TTR, for all the tested enzymes (Bst 2.0, optimized wild-type Bst59, Bst59-M794I, and Bst59-M794IH) in both 10× ThermoPol buffer and 2× Detect buffer, except for Bst 3.0 (in 10× ThermoPol buffer). Both Bst59-M794I and Bst59-M794IH had faster TTR than the other tested enzymes (Bst 2.0, Bst 3.0, optimized wild-type Bst59) (FIGS. 3A and 3B).

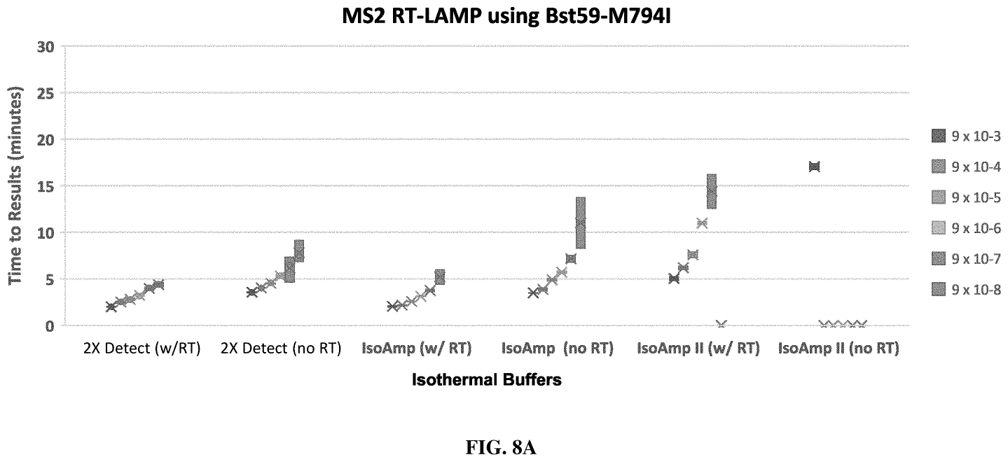

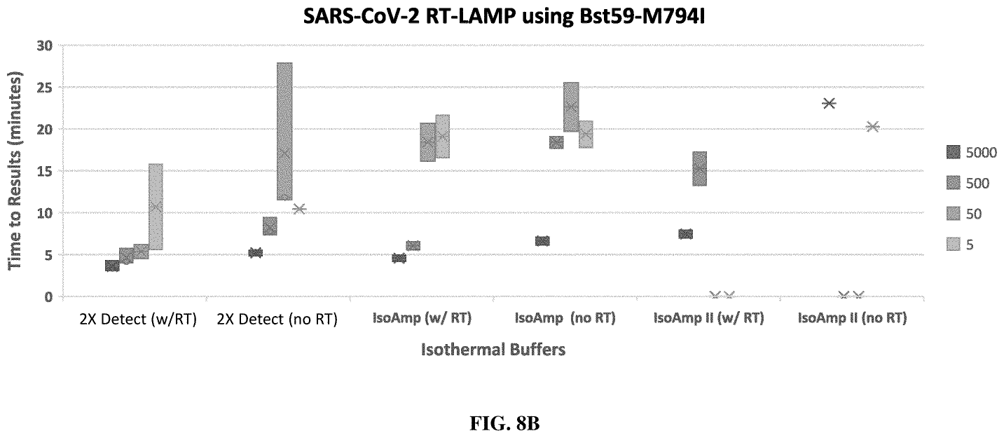

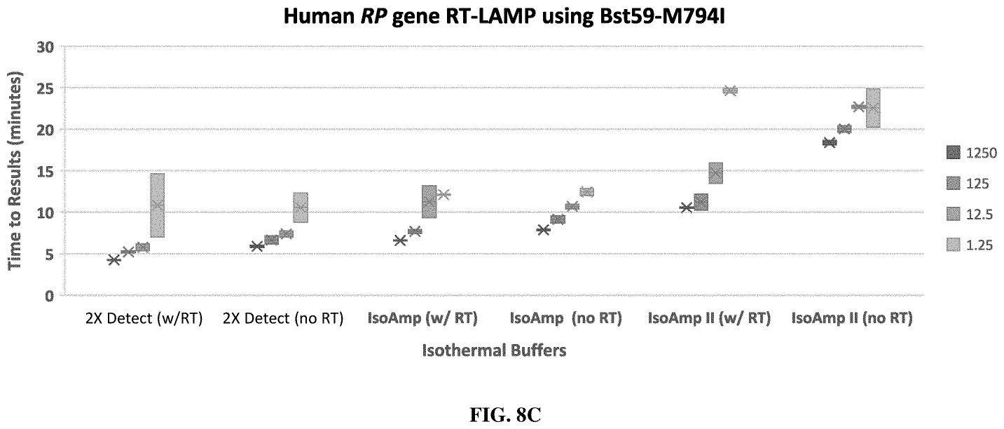

Similar results were observed upon replication of the experiments, and when using different RNA targets and/or different second enzymes having reverse transcriptase activity. Additionally, certain Bst variants of the disclosure were tested individually in different buffers, using different RNA targets, and both with and without the addition of a second enzyme having reverse transcriptase activity (see FIGS. 8A-8C, 9A-9B, 10A-10B, 11A-11B, 12A-12C, 13A-13B, and 14A-14B).

Collectively, the results shown in FIGS. 2A-2C, 3A-3E, 4, 5A-5B, 6A-6B, 8A-8C, 9A-9B, 10A-10B, 11A-11B, 12A-12C, 13A-13B and 14A-14B demonstrate that the Bst polymerase variants of the disclosure are capable of amplifying target MS2, SARS-CoV-2, and human RP RNA sequences without the addition of a second enzyme having RT activity at comparable or faster TTRs than commercially-available Bst polymerases (Bst 2.0, Bst 3.0, and Iso-004nd) and the optimized wild-type Bst59 polymerase (both with and without the addition of a second enzyme having RT activity). These results were consistent among various concentrations of target RNA and across various buffers and primer sets. Furthermore, the addition of a second enzyme having RT activity (either WarmStart® RTx Reverse Transcriptase or HIV RT) to the Bst polymerase variants of the disclosure improved assay performance in terms of both sensitivity (limit of detection; LOD) and speed (TTR), but was not necessary to amplify the target RNA templates using the Bst polymerase variants of the disclosure.

### Example 4: Additional Example Demonstrating a Bst Polymerase Variant of the Disclosure is Faster than an Existing State-of-the-Art DNA Polymerase

This Example demonstrates that the variant Bst59-A641T;M794I (SEQ ID NO: 39) was faster than Bst 2.0 (New England Biolabs, Cat. No. M0537) in RT-LAMP reactions targeting the SARS-CoV-2 genome.

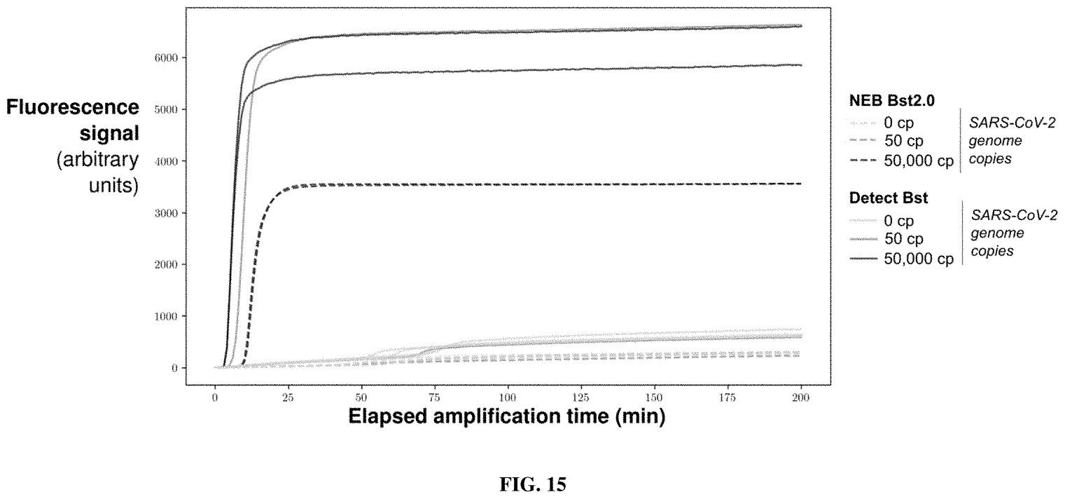

RT-LAMP master mixes comprising either the variant Bst59-A641T;M794I (referred to in this Example and FIG. 15 as “Detect Bst”) or Bst2.0 (referred to in this Example and FIG. 15 as “NEB Bst 2.0”) were prepared with an identical primer set targeting the SARS-CoV-2 genome. When a whole genome SARS-CoV-2 template at a given level (0 copies (cp), 50 copies, or 50,000 copies) and a fluorogenic molecular beacon probe specific for the intended product of nucleic acid amplification were included in the reaction, LAMP amplification proceeded. Production of nucleic acid products was tracked by generation of a fluorescence signal.

FIG. 15 shows a plot of fluorescence signal as a function of elapsed amplification time (minutes). As shown in FIG. 15, RT-LAMP amplification with Detect Bst was faster than with NEB Bst 2.0 at both the 50 cp and 50,000 cp levels. This demonstrates that RT-LAMP with Detect Bst is faster than with NEB Bst 2.0 across both high and low nucleic acid concentrations.

### Example 5: Additional Example Demonstrating a Bst Polymerase Variant of the Disclosure is Faster than an Existing State-of-the-Art DNA Polymerase

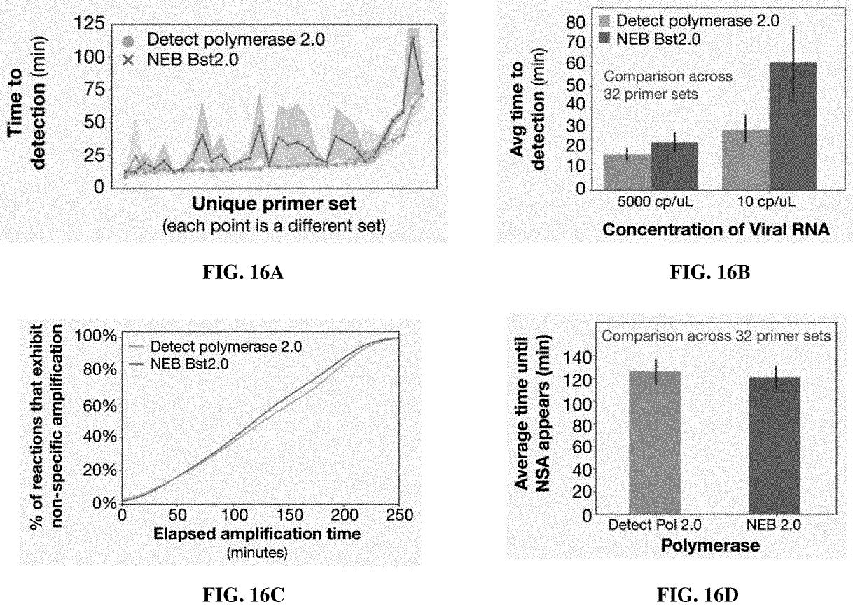

This Example further demonstrates that the variant Bst59-A641T;M794I (SEQ ID NO: 39) was consistently faster than Bst 2.0 (New England Biolabs, Cat. No. M0537) in RT-LAMP reactions targeting the SARS-CoV-2 genome. The variant Bst59-A641T;M794I (referred to in this Example and FIGS. 16A-16D as “Detect polymerase 2.0”) was compared with Bst 2.0 (referred to in this Example and FIGS. 16A-16D as “NEB Bst 2.0”) in RT-LAMP reactions using 32 different primer sets targeting the SARS-CoV-2 genome.

FIG. 16A shows a plot of the time to detection (minutes) for each of the 32 unique primer sets (i.e., each point represents a different primer set). As shown in FIG. 16A, the time to detection was consistently lower (i.e., faster) for Detect polymerase 2.0 than for NEB Bst 2.0.

FIG. 16B shows the average time to detection (minutes) across the 32 primer sets at two concentrations of Sars-CoV-2 RNA: 5000 cp/μL and 10 cp/μL. At both concentrations, the average time to detection for Detect polymerase 2.0 (left) was lower than for NEB Bst 2.0 (right). This demonstrates that even at low viral RNA concentrations, Detect polymerase 2.0 is faster than NEB Bst 2.0.

FIG. 16C shows a plot of percentage of reactions that exhibit non-specific amplification (NSA) as a function of elapsed amplification time (minutes). As shown in FIG. 16C, the specificity of Detect polymerase 2.0 was comparable to that of NEB Bst 2.0. That is, under otherwise identical chemical conditions, Detect polymerase 2.0 was not found to result in increased speed of non-specific amplification. Such increased speed (e.g., off-target reactions amplifying within a faster time window) could result in a loss of specificity (e.g., increased false positives).

FIG. 16D shows the average time until NSA appears (minutes) for Detect polymerase 2.0 (left) and NEB Bst 2.0 (right). As shown in FIG. 16D, the average time until NSA was comparable for Detect polymerase 2.0 and NEB Bst 2.0.

### Example 6: Expression and Purification of Bst777

This Example demonstrates the successful expression and purification of Bst777. FIG. 18 shows an image of an SDS-PAGE gel of Bst777 after it has been expressed and purified using a TALON® spin column (Takara Bio). As shown in FIG. 18, Bst777 expressed and gave a clean, pure expression product. FIG. 18 also shows C-terminal Bst59-A641T;M794I (SEQ ID NO: 156) (referred to as “C-term Bst59” in FIG. 18) and N-terminal Bst59-A641T;M794I (SEQ ID NO: 39) (referred to as “N-term Bst59” in FIG. 18).

### Example 7: Successful Use of Bst777 in a DNA Polymerase Primer Extension Assay

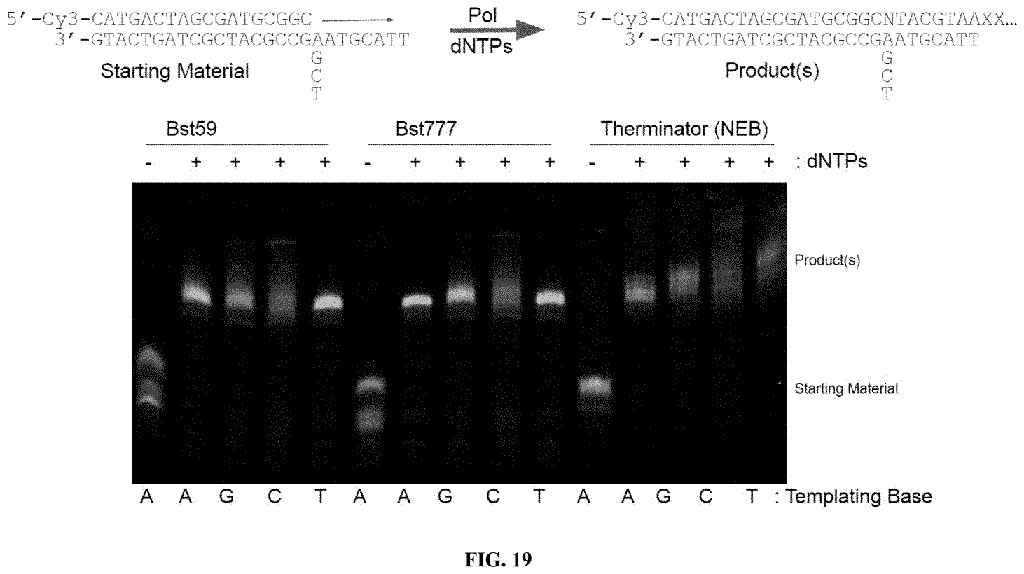

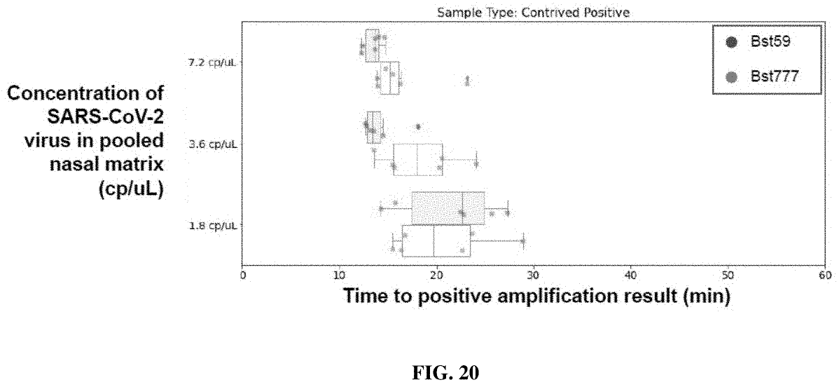

This Example demonstrates that Bst777 was successfully used in a primer extension assay. In this Example, a DNA polymerase primer extension assay was conducted using Bst59-A641T;M794I (SEQ ID NO: 39) (referred to as “Bst59” in this Example and FIG. 19), Bst777, or Therminator™ DNA polymerase (NEB). 1 μM Cy3-Primer/Template, about 2-5 μM DNA polymerase (i.e., either Bst59, Bst777, or Therminator™), and about 20 μM dNTPs in Thermopol buffer were incubated at 50° C. for 30 minutes. The reaction was quenched with formamide and EDTA. A 20% denaturing PAGE gel was run at 500 V for 1.5 hours and imaged on a Sapphire Gel Scanner. FIG. 19 shows an image of the resulting gel showing the products and starting material. From FIG. 19, it can be seen that Bst777 was successfully used to extend the primer in the primer extension assay.

### Example 8: Successful Use of Bst777 in RT-LAMP Reactions

This Example demonstrates that Bst777 (SEQ ID NO: 155) was successfully used in RT-LAMP reactions to amplify a portion of a SARS-CoV-2 nucleic acid sequence.

RT-LAMP reactions were run using either Bst59-A641T;M794I (SEQ ID NO: 39) (referred to as “Bst59” in this Example and FIG. 20) or Bst777 (SEQ ID NO: 155) with contrived positive samples comprising one of three concentrations of SARS-CoV-2 virus (1.8 cp/μL, 3.6 cp/μL, 7.2 cp/μL) in pooled nasal matrix samples. FIG. 20 shows the time to positive amplification result (minutes) for each RT-LAMP reaction. These results demonstrate that Bst777 was successfully used to amplify SARS-CoV-2 RNA across three different concentrations. That is, Bst777 not only expressed, folded, and purified well, but was also a functional strand-displacing polymerase with reasonably high activity levels in LAMP.

We claim:

1. A nucleic acid polymerase variant comprising one or more mutations relative to a wild-type Bacillus stearothermophilus (Bst) nucleic acid polymerase having an amino acid sequence as shown in SEQ ID NO: 1.

2. The nucleic acid polymerase variant of claim 1, wherein the variant further comprises a deletion of a 5′ to 3′ exonuclease domain having an amino acid sequence as shown in SEQ ID NO: 2, relative to the wild-type Bst nucleic acid polymerase having an amino acid sequence as shown in SEQ ID NO: 1.

3. The nucleic acid polymerase variant of claim 2, wherein the variant further comprises an N-terminal six-histidine tag having an amino acid sequence as shown in SEQ ID NO: 3, relative to the wild-type Bst nucleic acid polymerase having an amino acid sequence as shown in SEQ ID NO: 1.

4. The nucleic acid polymerase variant of claim 3, wherein the variant comprises an amino acid sequence as shown in SEQ ID NO: 4.

5. The nucleic acid polymerase variant of any one of claims 1-4, wherein the one or more mutations comprise an amino acid substitution.

6. The nucleic acid polymerase variant of any one of claims 1-5, wherein the one or more mutations are made in one or more amino acid positions selected from the group consisting of: N529, K584, N602, I630, A641, I659, V663, L664, I683, T685, I691, M703, R705, Q706, F712, V715, D720, F745, D777, S787, F788, M794, A802, R825, and D832, according to the numbering as shown in SEQ ID NO: 1.

7. The nucleic acid polymerase variant of any one of claims 1-6, wherein the one or more mutations are amino acid substitution(s) selected from the group consisting of: N529K, K584Y, N602A, N602L, I630G, A641T, I659K, V663I, L664M, I683V, T685K, I691V, M703L, R705V, Q706I, F712L, F712Y, V715M, D720A, F745Y, D777N, D777Q, S787R, F788H, F788R, M794I, A802G, R825H, and D832E, according to the numbering as shown in SEQ ID NO: 1.

8. The nucleic acid polymerase variant of any one of claims 3-7, wherein the variant has a single mutation, relative to the nucleic acid polymerase variant of claim 3 or claim 4.

9. The nucleic acid polymerase variant of any one of claims 3-7, wherein the variant has two mutations, relative to the nucleic acid polymerase variant of claim 3 or claim 4.

10. The nucleic acid polymerase variant of any one of claims 3-7, wherein the variant has three, four, five, six, seven, eight, nine, ten, eleven, twelve, thirteen, fourteen, or fifteen mutations, relative to the nucleic acid polymerase variant of claim 3 or claim 4.

11. The nucleic acid polymerase variant of claim 7, wherein the one or more mutations are selected from the group consisting of: M794I and R825H; N529K and M794I; K584Y and M794I; N602A and D832E; N602L and D832E; I630G and M794I; A641T and M794I; I659K and M794I; R705V and M794I; F712L and M794I; F712Y and M794I; D777Q and M794I; S787R and F788R; F788R and M794I; N529K, D777Q, and M794I; K584Y, D777Q, and M794I; I630G, D777Q, and M794I; A641T, D777Q, and M794I; I659K, D777Q, and M794I; R705V, D777Q, and M794I; F712L, D777Q, and M794I; F712Y, D777Q, and M794I; D777Q, S787R, and F788R; D777Q, F788R, and M794I; S787R, F788R, and M794I; F712Y, D777Q, F788R, and M794I; D777Q, S787R, F788R, and M794I; V663I, L664M, I683V, T685K, I691V, M703L, Q706I, V715M, F745Y, and A802G; and V663I, L664M, I683V, T685K, I691V, M703L, Q706I, V715M, F745Y, M794I, and A802G, according to the numbering as shown in SEQ ID NO: 1.

12. The nucleic acid polymerase variant of claim 7, wherein the one or more mutations is A641T, according to the numbering as shown in SEQ ID NO: 1.

13. The nucleic acid polymerase variant of claim 7, wherein the one or more mutations are A641T and M794I, according to the numbering as shown in SEQ ID NO: 1.

14. The nucleic acid polymerase variant of claim 7, wherein the one or more mutations is D777N, according to the numbering as shown in SEQ ID NO: 1.

15. The nucleic acid polymerase variant of claim 7, wherein the one or more mutations are S787R and F788R, according to the numbering as shown in SEQ ID NO: 1.

16. The nucleic acid polymerase variant of claim 7, wherein the one or more mutations is F788R, according to the numbering as shown in SEQ ID NO: 1.

17. The nucleic acid polymerase variant of claim 7, wherein the one or more mutations is M794I, according to the numbering as shown in SEQ ID NO: 1.

18. The nucleic acid polymerase variant of claim 7, wherein the one or more mutations are M794I and R825H, according to the numbering as shown in SEQ ID NO: 1.

19. The nucleic acid polymerase variant of claim 7, wherein the one or more mutations are V663I, L664M, I683V, T685K, I691V, M703L, Q706I, V715M, F745Y, and A802G, according to the numbering as shown in SEQ ID NO: 1.

20. The nucleic acid polymerase variant of claim 7, wherein the one or more mutations are V663I, L664M, I683V, T685K, I691V, M703L, Q706I, V715M, F745Y, M794I, and A802G, according to the numbering as shown in SEQ ID NO: 1.

21. The nucleic acid polymerase variant of any one of claims 1-20, wherein the one or more mutations result in faster amplification of a given concentration of a target nucleic acid relative to a polymerase selected from the group consisting of: the wild-type B st nucleic acid polymerase having an amino acid sequence as shown in SEQ ID NO: 1, a nucleic acid polymerase variant having an amino acid sequence as shown in SEQ ID NO: 4, Bst 2.0, Bst 3.0, and Geobacillus species SSD polymerase large fragment (GspSSD 2.0 LF).

22. The nucleic acid polymerase variant of any one of claims 1-21, wherein the variant amplifies a target nucleic acid in 19 minutes or less, 18 minutes or less, 17 minutes or less, 16 minutes or less, 15 minutes or less, 14 minutes or less, 13 minutes or less, 12 minutes or less, 11 minutes or less, 10 minutes or less, 9 minutes or less, 8 minutes or less, 7 minutes or less, 6 minutes or less, 5 minutes or less, 4 minutes or less, or 3 minutes or less.

23. The nucleic acid polymerase variant of any one of claims 1-22, wherein the variant has increased reverse transcriptase activity for a given concentration of a target nucleic acid, relative to a polymerase selected from the group consisting of: the wild-type Bst nucleic acid polymerase having an amino acid sequence as shown in SEQ ID NO: 1, a nucleic acid polymerase variant having an amino acid sequence as shown in SEQ ID NO: 4, Bst 2.0, Bst 3.0, and Geobacillus species SSD polymerase large fragment (GspSSD 2.0 LF).

24. The nucleic acid polymerase variant of any one of claims 21-23, wherein the target nucleic acid is a ribonucleic acid (RNA) and wherein amplification of the target nucleic acid occurs without a second enzyme having reverse transcriptase activity.

25. The nucleic acid polymerase variant of any one of claims 21-23, wherein the target nucleic acid is an RNA and wherein amplification of the target RNA occurs with a second enzyme having reverse transcriptase activity.

26. The nucleic acid polymerase variant of claim 24 or claim 25, wherein the target RNA is RNA from MS2, SARS-CoV-2, or human ribonuclease P (RP).

27. The nucleic acid polymerase variant of any one of claims 21-23, wherein the target nucleic acid is a deoxyribonucleic acid (DNA).

28. The nucleic acid polymerase variant of claim 27, wherein the target DNA is DNA from Aeromonas.

29. A nucleic acid polymerase variant comprising a polypeptide having at least 80%, at least 85%, at least 90%, at least 95%, or at least 99% sequence identity to an amino acid sequence as shown in any one of SEQ ID NOs: 6-63.

30. The nucleic acid polymerase variant of claim 29, wherein the variant has an amino acid sequence as shown in any one of SEQ ID NOs: 6-63.

31. A nucleic acid polymerase variant comprising a polypeptide having at least 80%, at least 85%, at least 90%, at least 95%, or at least 99% sequence identity to an amino acid sequence as shown in SEQ ID NO: 39.

32. The nucleic acid polymerase variant of claim 31, wherein the variant has an amino acid sequence as shown in SEQ ID NO: 39.

33. A nucleic acid polymerase variant comprising one or more mutations relative to a B st polymerase having an amino acid as shown in SEQ ID NO: 1 or SEQ ID NO: 4, wherein the one or more mutations comprise S299A, D300K, I301M, D302A, Y303F, I305L, V306A, E308R, S312E, I313M, S315A, E317K, L325V, S327E, K331D, L335V, F337I, I339V, A340V, N345R, I346L, T350P, D351E, S355A, S356D, S357P, L358Q, T360V, Q361A, E364G, S367T, V372M, G375S, I379A, S381A, Q385K, Q388E, R390C, Q393S, I398L, S400A, N404D, S406A, S408G, T409V, E410D, S414A, I415A, T418M, T422E, D423A, Q425R, S426P, I430V, Q437R, K438A, I439V, R457W, Q461R, D462P, I464L, C465D, D466E, Q468R, E469R, Y473D, S474R, F476L, T477V, D478E, L481Q, K514R, A641T, Q750R, K753E, D755N, and/or M794I.

34. The nucleic acid polymerase variant of claim 33, wherein the one or more mutations comprise 77 mutations.

35. The nucleic acid polymerase variant of any one of claims 33-34, wherein the one or more mutations comprise S299A, D300K, I301M, D302A, Y303F, I305L, V306A, E308R, S312E, I313M, S315A, E317K, L325V, S327E, K331D, L335V, F337I, I339V, A340V, N345R, I346L, T350P, D351E, S355A, S356D, S357P, L358Q, T360V, Q361A, E364G, S367T, V372M, G375S, I379A, S381A, Q385K, Q388E, R390C, Q393S, I398L, S400A, N404D, S406A, S408G, T409V, E410D, S414A, I415A, T418M, T422E, D423A, Q425R, S426P, I430V, Q437R, K438A, I439V, R457W, Q461R, D462P, I464L, C465D, D466E, Q468R, E469R, Y473D, S474R, F476L, T477V, D478E, L481Q, K514R, A641T, Q750R, K753E, D755N, and M794I.

36. A nucleic acid polymerase variant comprising a polypeptide having at least 80%, at least 85%, at least 90%, at least 95%, or at least 99% sequence identity to an amino acid sequence as shown in SEQ ID NO: 155.

37. The nucleic acid polymerase variant of claim 36, wherein the variant has an amino acid sequence as shown in SEQ ID NO: 155.

38. A method of detecting a target nucleic acid sequence, the method comprising:
(i) obtaining a biological sample from a subject;
(ii) performing a nucleic acid amplification reaction configured to amplify the target nucleic acid sequence using a nucleic acid polymerase variant according to any one of claims 1-30, and
(iii) detecting the presence or absence of the target nucleic acid sequence.

39. The method of claim 38, wherein the target nucleic acid sequence is a DNA sequence or an RNA sequence.

40. The method of claim 38 or claim 39, wherein the subject is a human, non-human primate, or mouse subject.

41. The method of any one of claims 38-40, wherein the target nucleic acid sequence is a DNA sequence, and wherein the nucleic acid amplification reaction comprises LAMP.

42. The method of any one of claims 38-41, wherein the target nucleic acid sequence is an RNA sequence, and wherein the nucleic acid amplification reaction comprises RT-LAMP.

43. The method of claim 42, further comprising a step of adding a second enzyme having reverse transcriptase activity to the nucleic acid amplification reaction.

44. The method of any one of claims 38-43, wherein the target nucleic acid sequence is detected using a lateral flow assay (LFA) strip, a colorimetric assay, a CRISPR/Cas method of detection, or is directly detected using hybridization.

45. The method of any one of claims 38-44, wherein the biological sample comprises a mucus, saliva, sputum, urine, blood, or cell scraping sample.

46. The method of any one of claims 38-45, wherein the biological sample comprises a vaginal or semen sample.

47. A kit for the detection of a target nucleic acid sequence comprising a nucleic acid polymerase variant according to any one of claims 1-46.

48. The kit of claim 47, further comprising a second enzyme having reverse transcriptase activity.

49. A method of making a kit for the detection of a target nucleic acid sequence comprising a nucleic acid polymerase variant according to any one of claims 1-46.

50. The method of claim 49, wherein the kit further comprises a second enzyme having reverse transcriptase activity.


SEQUENCE LISTING (156 sequences)

SEQ ID NO: 1 - protein, 878 aa, Geobacillus sp.
MKKKLVLIDGNSIAYRAFFALPLLHNDKGIHTNAVYGFTMMLMKILEEEKPTHMLVAFDAGKTTFRHKTFEEYKGGRQQTPPELSEQFPLLRELLDAYRIRFYELENYEADDIIGTLCTKAEKEGFEVKVISGDRDLTQLASDHVTVDITKKGITDVESYTPETVQEKYGLTPEQIIDLKGLMGDKSDNIPGVPGIGEKTALKLLKEFGTIENILDSIEQVSGNKLRENLAKYRDIALMSKQLATILRDAPVDLSLEDIEYNGYDVDKVIALFKELGFNSLLEKMAAPQGEEETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 2 - protein, 292 aa, Geobacillus sp.
MKKKLVLIDGNSIAYRAFFALPLLHNDKGIHTNAVYGFTMMLMKILEEEKPTHMLVAFDAGKTTFRHKTFEEYKGGRQQTPPELSEQFPLLRELLDAYRIRFYELENYEADDIIGTLCTKAEKEGFEVKVISGDRDLTQLASDHVTVDITKKGITDVESYTPETVQEKYGLTPEQIIDLKGLMGDKSDNIPGVPGIGEKTALKLLKEFGTIENILDSIEQVSGNKLRENLAKYRDIALMSKQLATILRDAPVDLSLEDIEYNGYDVDKVIALFKELGFNSLLEKMAAPQGEE

SEQ ID NO: 3 - protein, 8 aa, synthetic construct
MHHHHHHG

SEQ ID NO: 4 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 5 - protein, 594 aa, synthetic construct
MHHHHHHAEGEKPLEEMEFAIVDVITEEMLADKAALVVEVMEENYHDAPIVGIALVNEHGRFFMRPETALADSQFLAWLADETKKKSMFDAKRAVVALKWKGIELRGVAFDLLLAAYLLNPAQDAGDIAAVAKMKQYEAVRSDEAVYGKGVKRSLPDEQTLAEHLVRKAAAIWALEQPFMDDLRNNEQDQLLTKLEQPLAAILAEMEFTGVNVDTKRLEQMGSELAEQLRAIEQRIYELAGQEFNINSPKQLGVILFEKLQLPVLKKTKTGYSTSADVLEKLAPHHEIVENILHYRQLGKLQSTYIEGLLKVVRPDTGKVHTMFNQALTQTGRLSSAEPNLQNIPIRLEEGRKIRQAFVPSEPDWLIFAADYSQIELRVLAHIADDDNLIEAFQRDLDIHTKTAMDIFHVSEEEVTANMRRQAKAVNFGIVYGISDYGLAQNLNITRKEAAEFIERYFASFPGVKQYMENIVQEAKQKGYVTTLLHRRRYLPDITSRNFNVRSFAERTAMNTPIQGSAADIIKKAMIDLAARLKEEQLQARLLLQVHDELILEAPKEEIERLCELVPEVMEQAVTLRVPLKVDYHYGPTWYDAK

SEQ ID NO: 6 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFKINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 7 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGYLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 8 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTAKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 9 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTLKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 10 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPGRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 11 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQTFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 12 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQKELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 13 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRILAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 14 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVMAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 15 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDVHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 16 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHKKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 17 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDVFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 18 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDVFHVNEDEVTPNLRRQAKAVNFGIVYGISDYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 19 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRVQAKAVNFGIVYGISDYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 20 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDVFHVNEDEVTPNMRRIAKAVNFGIVYGISDYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 21 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNLGIVYGISDYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 22 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNYGIVYGISDYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 23 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDVFHVNEDEVTPNMRRQAKAVNFGIMYGISDYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 24 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISAYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 25 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPNITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 26 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPQITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 27 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRRFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 28 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSHAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 29 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSRAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 30 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAINTPIQGSAADIIKKAMIDLSNRLKKENMKARMLLQVHDELILEAPKEEVERLQQIVPEVMENAVQLRVPLKVDYHFGPTWYDAK

SEQ ID NO: 31 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKAHMLLQVHDELILEAPKEEVERLQQIVPEVMENAVQLRVPLKVDYHFGPTWYDAK

SEQ ID NO: 32 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKARMLLQVHEELILEAPKEEVERLQQIVPEVMENAVQLRVPLKVDYHFGPTWYDAK

SEQ ID NO: 33 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAINTPIQGSAADIIKKAMIDLSNRLKKENMKAHMLLQVHDELILEAPKEEVERLQQIVPEVMENAVQLRVPLKVDYHFGPTWYDAK

SEQ ID NO: 34 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFKINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAINTPIQGSAADIIKKAMIDLSNRLKKENMKARMLLQVHDELILEAPKEEVERLQQIVPEVMENAVQLRVPLKVDYHFGPTWYDAK

SEQ ID NO: 35 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGYLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAINTPIQGSAADIIKKAMIDLSNRLKKENMKARMLLQVHDELILEAPKEEVERLQQIVPEVMENAVQLRVPLKVDYHFGPTWYDAK

SEQ ID NO: 36 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTAKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKARMLLQVHEELILEAPKEEVERLQQIVPEVMENAVQLRVPLKVDYHFGPTWYDAK

SEQ ID NO: 37 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTLKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAMNTPIQGSAADIIKKAMIDLSNRLKKENMKARMLLQVHEELILEAPKEEVERLQQIVPEVMENAVQLRVPLKVDYHFGPTWYDAK

SEQ ID NO: 38 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPGRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAINTPIQGSAADIIKKAMIDLSNRLKKENMKARMLLQVHDELILEAPKEEVERLQQIVPEVMENAVQLRVPLKVDYHFGPTWYDAK

SEQ ID NO: 39 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQTFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAINTPIQGSAADIIKKAMIDLSNRLKKENMKARMLLQVHDELILEAPKEEVERLQQIVPEVMENAVQLRVPLKVDYHFGPTWYDAK

SEQ ID NO: 40 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQKELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAINTPIQGSAADIIKKAMIDLSNRLKKENMKARMLLQVHDELILEAPKEEVERLQQIVPEVMENAVQLRVPLKVDYHFGPTWYDAK

SEQ ID NO: 41 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRVQAKAVNFGIVYGISDYGLSQNLNITRKEASEFIKRYFEIFPGVKQYMKDIVQEAKQKGYVTTLLHRRRYLPDITSRNFNLRSFAERTAINTPIQGSAADIIKKAMIDLSNRLKKENMKARMLLQVHDELILEAPKEEVERLQQIVPEVMENAVQLRVPLKVDYHFGPTWYDAK

SEQ ID NO: 42 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNLGIVYGISDYGLSQNLNITRKEASEFIKRYFEIFPGVKQYMKDIVQEAKQKGYVTTLLHRRRYLPDITSRNFNLRSFAERTAINTPIQGSAADIIKKAMIDLSNRLKKENMKARMLLQVHDELILEAPKEEVERLQQIVPEVMENAVQLRVPLKVDYHFGPTWYDAK

SEQ ID NO: 43 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNYGIVYGISDYGLSQNLNITRKEASEFIKRYFEIFPGVKQYMKDIVQEAKQKGYVTTLLHRRRYLPDITSRNFNLRSFAERTAINTPIQGSAADIIKKAMIDLSNRLKKENMKARMLLQVHDELILEAPKEEVERLQQIVPEVMENAVQLRVPLKVDYHFGPTWYDAK

SEQ ID NO: 44 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPQITSRNFNLRSFAERTAINTPIQGSAADIIKKAMIDLSNRLKKENMKARMLLQVHDELILEAPKEEVERLQQIVPEVMENAVQLRVPLKVDYHFGPTWYDAK

SEQ ID NO: 45 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRRRAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 46 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSRAERTAINTPIQGSAADIIKKAMIDLSNRLKKENMKARMLLQVHDELILEAPKEEVERLQQIVPEVMENAVQLRVPLKVDYHFGPTWYDAK

SEQ ID NO: 47 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFKINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPQITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 48 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGYLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPQITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 49 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPGRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPQITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 50 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQTFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPQITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 51 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQKELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPQITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 52 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRVQAKAVNFGIVYGISDYGLSQNLNITRKEASEFIKRYFEIFPGVKQYMKDIVQEAKQKGYVTTLLHRRRYLPQITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 53 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNLGIVYGISDYGLSQNLNITRKEASEFIKRYFEIFPGVKQYMKDIVQEAKQKGYVTTLLHRRRYLPQITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 54 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNYGIVYGISDYGLSQNLNITRKEASEFIKRYFEIFPGVKQYMKDIVQEAKQKGYVTTLLHRRRYLPQITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 55 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPQITSRNFNLRRFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 56 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPQITSRNFNLRSRAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 57 - protein, 594 aa, synthetic construct; identical to SEQ ID NO: 45

SEQ ID NO: 58 - protein, 594 aa, synthetic construct; identical to SEQ ID NO: 54

SEQ ID NO: 59 - protein, 594 aa, synthetic construct; identical to SEQ ID NO: 55

SEQ ID NO: 60 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRIMAHIANDENLIDAFRHDLDVHKKTAMDVFHVNEDEVTPNLRRIAKAVNFGIMYGISDYGLSQNLNITRKEASEFIKRYFEIYPGVKQYMKDIVQEAKQKGYVTTLLHRRRYLPDITSRNFNLRSFAERTAMNTPIQGSGADIIKKAMIDLSNRLKKENMKARMLLQVHDELILEAPKEEVERLQQIVPEVMENAVQLRVPLKVDYHFGPTWYDAK

SEQ ID NO: 61 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRIMAHIANDENLIDAFRHDLDVHKKTAMDVFHVNEDEVTPNLRRIAKAVNFGIMYGISDYGLSQNLNITRKEASEFIKRYFEIYPGVKQYMKDIVQEAKQKGYVTTLLHRRRYLPDITSRNFNLRSFAERTAINTPIQGSGADIIKKAMIDLSNRLKKENMKARMLLQVHDELILEAPKEEVERLQQIVPEVMENAVQLRVPLKVDYHFGPTWYDAK

SEQ ID NO: 62 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIYPGVKQYMKDIVQEAKQKGYVTTLLHRRRYLPDITSRNFNLRSFAERTAMNTPIQGSAADIIKKAMIDLSNRLKKENMKARMLLQVHDELILEAPKEEVERLQQIVPEVMENAVQLRVPLKVDYHFGPTWYDAK

SEQ ID NO: 63 - protein, 594 aa, synthetic construct
MHHHHHHGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQAFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAMNTPIQGSGADIIKKAMIDLSNRLKKENMKARMLLQVHDELILEAPKEEVERLQQIVPEVMENAVQLRVPLKVDYHFGPTWYDAK

SEQ ID NO: 64 - DNA, 45 bp, synthetic construct
  source 1..45: mol_type = other DNA
gaactggcgggtcaagaatttaaaatcaattctccgaagcaactg

SEQ ID NO: 65 - DNA, 45 bp, synthetic construct
  source 1..45: mol_type = other DNA
cagttgcttcggagaattgattttaaattcttgacccgccagttc

SEQ ID NO: 66 - DNA, 48 bp, synthetic construct
  source 1..48: mol_type = other DNA
cctgcactatcgccagttggggtacctgcagtccacctacatcgaagg

SEQ ID NO: 67 - DNA, 48 bp, synthetic construct
  source 1..48: mol_type = other DNA
ccttcgatgtaggtggactgcaggtaccccaactggcgatagtgcagg

SEQ ID NO: 68 - DNA, 37 bp, synthetic construct
  source 1..37: mol_type = other DNA
gttgggtaagctgcagaatacgtacatcgaaggcttg

SEQ ID NO: 69 - DNA, 37 bp, synthetic construct
  source 1..37: mol_type = other DNA
caagccttcgatgtacgtattctgcagcttacccaac

SEQ ID NO: 70 - DNA, 46 bp, synthetic construct
  source 1..46: mol_type = other DNA
gctgaaagttgttcatcatgacacggcgaaagtgcacactattttc

SEQ ID NO: 71 - DNA, 46 bp, synthetic construct
  source 1..46: mol_type = other DNA
gaaaatagtgtgcactttcgccgtgtcatgatgaacaactttcagc

SEQ ID NO: 72 - DNA, 46 bp, synthetic construct
  source 1..46: mol_type = other DNA
gctgaaagttgttcatcatgacacgctgaaagtgcacactattttc

SEQ ID NO: 73 - DNA, 46 bp, synthetic construct
  source 1..46: mol_type = other DNA
gaaaatagtgtgcactttcagcgtgtcatgatgaacaactttcagc

SEQ ID NO: 74 - DNA, 46 bp, synthetic construct
  source 1..46: mol_type = other DNA
gccgaatttgcagaacatccccggccgtctggaagagggccgtaag

SEQ ID NO: 75 - DNA, 46 bp, synthetic construct
  source 1..46: mol_type = other DNA
cttacggccctcttccagacggccggggatgttctgcaaattcggc

SEQ ID NO: 76 - DNA, 43 bp, synthetic construct
  source 1..43: mol_type = other DNA
gagggccgtaagattcgccaaacgttcgtcccgtctgagccgg

SEQ ID NO: 77 - DNA, 43 bp, synthetic construct
  source 1..43: mol_type = other DNA
ccggctcagacgggacgaacgtttggcgaatcttacggccctc

SEQ ID NO: 78 - DNA, 49 bp, synthetic construct
  source 1..49: mol_type = other DNA
ctttagcgccgactatagccagaaagagctccgtgtcctggcgcatatc

SEQ ID NO: 79 - DNA, 49 bp, synthetic construct
  source 1..49: mol_type = other DNA
gatatgcgccaggacacggagctctttctggctatagtcggcgctaaag

SEQ ID NO: 80 - DNA, 46 bp, synthetic construct
  source 1..46: mol_type = other DNA
gaagtgaccccgaacatgcgtgtacaggcaaaagccgtgaattttg

SEQ ID NO: 81 - DNA, 46 bp, synthetic construct
  source 1..46: mol_type = other DNA
caaaattcacggcttttgcctgtacacgcatgttcggggtcacttc

SEQ ID NO: 82 - DNA, 49 bp, synthetic construct
  source 1..49: mol_type = other DNA
cgccgtcaggcaaaagccgttaacctgggtatcgtgtatggtatcagcg

SEQ ID NO: 83 - DNA, 49 bp, synthetic construct
  source 1..49: mol_type = other DNA
cgctgataccatacacgatacccaggttaacggcttttgcctgacggcg

SEQ ID NO: 84 - DNA, 49 bp, synthetic construct
  source 1..49: mol_type = other DNA
cgccgtcaggcaaaagccgttaactatggtatcgtgtatggtatcagcg

SEQ ID NO: 85 - DNA, 49 bp, synthetic construct
  source 1..49: mol_type = other DNA
cgctgataccatacacgataccatagttaacggcttttgcctgacggcg

SEQ ID NO: 86 - DNA, 36 bp, synthetic construct
  source 1..36: mol_type = other DNA
gtgtatggtatcagcgcatatggtctgagccaaaac

SEQ ID NO: 87 - DNA, 36 bp, synthetic construct
  source 1..36: mol_type = other DNA
gttttggctcagaccatatgcgctgataccatacac

SEQ ID NO: 88 - DNA, 43 bp, synthetic construct
  source 1..43: mol_type = other DNA
cagacgttatctgccgaatatcactagtcgcaactttaatctg

SEQ ID NO: 89 - DNA, 43 bp, synthetic construct
  source 1..43: mol_type = other DNA
cagattaaagttgcgactagtgatattcggcagataacgtctg

SEQ ID NO: 90 - DNA, 43 bp, synthetic construct
  source 1..43: mol_type = other DNA
cagacgttatctgccgcagatcactagtcgcaactttaatctg

SEQ ID NO: 91 - DNA, 43 bp, synthetic construct
  source 1..43: mol_type = other DNA
cagattaaagttgcgactagtgatctgcggcagataacgtctg

SEQ ID NO: 92 - DNA, 42 bp, synthetic construct
  source 1..42: mol_type = other DNA
cgcaactttaatctgcgtcggcgcgccgagcgtacggcgatg

SEQ ID NO: 93 - DNA, 42 bp, synthetic construct
  source 1..42: mol_type = other DNA
catcgccgtacgctcggcgcgccgacgcagattaaagttgcg

SEQ ID NO: 94 - DNA, 37 bp, synthetic construct
  source 1..37: mol_type = other DNA
caactttaatctgcgcagccatgcggagcgtacggcg

SEQ ID NO: 95 - DNA, 37 bp, synthetic construct
  source 1..37: mol_type = other DNA
cgccgtacgctccgcatggctgcgcagattaaagttg

SEQ ID NO: 96 - DNA, 37 bp, synthetic construct
  source 1..37: mol_type = other DNA
caactttaatctgcgttcacgtgcggagcgtacggcg

SEQ ID NO: 97 - DNA, 37 bp, synthetic construct
  source 1..37: mol_type = other DNA
cgccgtacgctccgcacgtgaacgcagattaaagttg

SEQ ID NO: 98 - DNA, 31 bp, synthetic construct
  source 1..31: mol_type = other DNA
ggagcgtacggcgattaatacgccgattcag

SEQ ID NO: 99 - DNA, 31 bp, synthetic construct
  source 1..31: mol_type = other DNA
ctgaatcggcgtattaatcgccgtacgctcc

SEQ ID NO: 100 - DNA, 33 bp, synthetic construct
  source 1..33: mol_type = other DNA
gccgagcgtacggcgattaatacgccgattcag

SEQ ID NO: 101 - DNA, 33 bp, synthetic construct
  source 1..33: mol_type = other DNA
ctgaatcggcgtattaatcgccgtacgctcggc

SEQ ID NO: 102 - DNA, 47 bp, synthetic construct
  source 1..47: mol_type = other DNA
gaaaaaagaaaatatgaaagcgcatatgctgctgcaagtccacgacg

SEQ ID NO: 103 - DNA, 47 bp, synthetic construct
  source 1..47: mol_type = other DNA
cgtcgtggacttgcagcagcatatgcgctttcatattttcttttttc

SEQ ID NO: 104 - DNA, 45 bp, synthetic construct
  source 1..45: mol_type = other DNA
cgcgcatgctgctgcaagttcatgaagaactgattttagaggcgc

SEQ ID NO: 105 - DNA, 45 bp, synthetic construct
  source 1..45: mol_type = other DNA
gcgcctctaaaatcagttcttcatgaacttgcagcagcatgcgcg

SEQ ID NO: 106 - DNA, 25 bp, synthetic construct
  source 1..25: mol_type = other DNA
cgcggacatcatcaaaaaagcaatg

SEQ ID NO: 107 - DNA, 23 bp, synthetic construct
  source 1..23: mol_type = other DNA
ctcaatctggctatagtcggcgc

SEQ ID NO: 108 - DNA, 32 bp, synthetic construct
  source 1..32: mol_type = other DNA
ccgactatagccagattgagcttcgtattatg

SEQ ID NO: 109 - DNA, 22 bp, synthetic construct
  source 1..22: mol_type = other DNA
cttttttgatgatgtccgcgcc

SEQ ID NO: 110 - DNA, 24 bp, synthetic construct
  source 1..24: mol_type = other DNA
ggacgaatgcttggcgaatcttac

SEQ ID NO: 111 - DNA, 22 bp, synthetic construct
  source 1..22: mol_type = other DNA
gaaaattccggacgagccagtg

SEQ ID NO: 112 - DNA, 23 bp, synthetic construct
  source 1..23: mol_type = other DNA
gccttgtggtaattgctctccag

SEQ ID NO: 113 - DNA, 23 bp, synthetic construct
  source 1..23: mol_type = other DNA
gactaccactttggtccgacctg

SEQ ID NO: 114 - DNA, 19 bp, synthetic construct
  source 1..19: mol_type = other DNA
gttcgtgcacacagcccag

SEQ ID NO: 115 - DNA, 27 bp, synthetic construct
  source 1..27: mol_type = other DNA
gaaaaagccgtttctgtaatgaaggag

SEQ ID NO: 116 - DNA, 19 bp, synthetic construct
  source 1..19: mol_type = other DNA
ctgtttcgcctcttgcacg

SEQ ID NO: 117 - DNA, 21 bp, synthetic construct
  source 1..21: mol_type = other DNA
cagtccagttacgctggagtc

SEQ ID NO: 118 - DNA, 26 bp, synthetic construct
  source 1..26: mol_type = other DNA
ggtcaggtatgatttaaatggtcagt

SEQ ID NO: 119 - DNA, 19 bp, synthetic construct
  source 1..19: mol_type = other DNA
gctagttattgctcagcgg

SEQ ID NO: 120 - DNA, 30 bp, synthetic construct
  source 1..30: mol_type = other DNA
tgtcatgggatccggatgttttacaaacca

SEQ ID NO: 121 - DNA, 21 bp, synthetic construct
  source 1..21: mol_type = other DNA
acggcgcacattggtctcgga

SEQ ID NO: 122 - DNA, 24 bp, synthetic construct
  source 1..24: mol_type = other DNA
cgccagggttttcccagtcacgac

SEQ ID NO: 123 - DNA, 18 bp, synthetic construct
  source 1..18: mol_type = other DNA
cggtggacaaattgtcac

SEQ ID NO: 124 - DNA, 22 bp, synthetic construct
  source 1..22: mol_type = other DNA
cttctctggatttaacacactt

SEQ ID NO: 125 - DNA, 28 bp, synthetic construct
  source 1..28: mol_type = other DNA
ttacaagcttaaagaatgtctgaacact

SEQ ID NO: 126 - DNA, 27 bp, synthetic construct
  source 1..27: mol_type = other DNA
ttgaatttaggtgaaacatttgtcacg

SEQ ID NO: 127 - DNA, 47 bp, synthetic construct
  source 1..47: mol_type = other DNA
tcagcacacaaagccaaaaatttatctgtgcaaaggaaattaaggag

SEQ ID NO: 128 - DNA, 45 bp, synthetic construct
  source 1..45: mol_type = other DNA
tattggtggagctaaacttaaagccctgtacaatccctttgagtg

SEQ ID NO: 129 - DNA, 51 bp, synthetic construct
  source 1..51: mol_type = other DNA
tcagcacacaaagccaaaaatttatttttctgtgcaaaggaaattaaggag

SEQ ID NO: 130 - DNA, 49 bp, synthetic construct
  source 1..49: mol_type = other DNA
tattggtggagctaaacttaaagccttttctgtacaatccctttgagtg

SEQ ID NO: 131 - DNA, 18 bp, synthetic construct
  source 1..18: mol_type = other DNA
tgcttcagtcagctgatg

SEQ ID NO: 132 - DNA, 22 bp, synthetic construct
  source 1..22: mol_type = other DNA
ttaaattgtcatcttcgtcctt

SEQ ID NO: 133 - DNA, 41 bp, synthetic construct
  source 1..41: mol_type = other DNA
tcagtactagtgcctgtgcccacaatcgtttttaaacgggt

SEQ ID NO: 134 - DNA, 43 bp, synthetic construct
  source 1..43: mol_type = other DNA
tcgtatacagggcttttgacatctatcttggaagcgacaacaa

SEQ ID NO: 135 - DNA, 17 bp, synthetic construct
  source 1..17: mol_type = other DNA
ctgcacttacaccgcaa

SEQ ID NO: 136 - DNA, 22 bp, synthetic construct
  source 1..22: mol_type = other DNA
gtagctggttttgctaaattcc

SEQ ID NO: 137 - DNA, 17 bp, synthetic construct
  source 1..17: mol_type = other DNA
ttgatgagctggagcca

SEQ ID NO: 138 - DNA, 18 bp, synthetic construct
  source 1..18: mol_type = other DNA
caccctcaatgcagagtc

SEQ ID NO: 139 - DNA, 20 bp, synthetic construct
  source 1..20: mol_type = other DNA
atgtggatggctgagttgtt

SEQ ID NO: 140 - DNA, 20 bp, synthetic construct
  source 1..20: mol_type = other DNA
catgctgagtactggacctc

SEQ ID NO: 141 - DNA, 41 bp, synthetic construct
  source 1..41: mol_type = other DNA
gtgtkaccctgaagactcggttttagccactgactcggatc

SEQ ID NO: 142 - DNA, 45 bp, synthetic construct
  source 1..45: mol_type = other DNA
cctccgtgatatggctcttcgtttttttcttacatggctctggtc

SEQ ID NO: 143 - DNA, 20 bp, synthetic construct
  source 1..20: mol_type = other DNA
tgtcatgggatccggatgtt

SEQ ID NO: 144 - DNA, 20 bp, synthetic construct
  source 1..20: mol_type = other DNA
caatagagccgctctcagag

SEQ ID NO: 145 - DNA, 19 bp, synthetic construct
  source 1..19: mol_type = other DNA
ccagagaggaggttgccaa

SEQ ID NO: 146 - DNA, 19 bp, synthetic construct
  source 1..19: mol_type = other DNA
tgcaggatgcagcgcctta

SEQ ID NO: 147 - DNA, 41 bp, synthetic construct
  source 1..41: mol_type = other DNA
gcccaaacaacgacgatcggtaaaaccagcatccgtagcct

SEQ ID NO: 148 - DNA, 40 bp, synthetic construct
  source 1..40: mol_type = other DNA
gcacgttctccaacggtgctggttgcttgttcagcgaact

SEQ ID NO: 149 - DNA, 21 bp, synthetic construct
  source 1..21: mol_type = other DNA
gctgctgataattcggctgtt

SEQ ID NO: 150 - DNA, 24 bp, synthetic construct
  source 1..24: mol_type = other DNA
ctcttaatcgaatctccaggatct

SEQ ID NO: 151 - DNA, 24 bp, synthetic construct
  source 1..24: mol_type = other DNA
agagcggggtttaaatatattgaa

SEQ ID NO: 152 - DNA, 25 bp, synthetic construct
  source 1..25: mol_type = other DNA
gctacttttttcactgattcctgtt

SEQ ID NO: 153 - DNA, 47 bp, synthetic construct
  source 1..47: mol_type = other DNA
gcactttatgaacgcttgaaagaaataaagctattactgccccctcg

SEQ ID NO: 154 - DNA, 43 bp, synthetic construct
  source 1..43: mol_type = other DNA
aacttgattgttttcattcctgacttgagcggtatgctgtcgc

SEQ ID NO: 155 - protein, 600 aa, synthetic construct
MHHHHHHGSESPSSEEEKPLAKMAFTLADRVTEEMLADKAALVVEVVEENYHDAPIVGIAVVNEHGRFFLRPETALADPQFVAWLGDETKKKSMFDSKRAAVALKWKGIELCGVSFDLLLAAYLLDPAQGVDDVAAAAKMKQYEAVRPDEAVYGKGAKRAVPDEPVLAEHLVRKAAAIWALERPFLDELRRNEQDRLLVELEQPLSSILAEMEFTGVKIDVKRLKEMGEELTEQLREIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQTFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKRYMENIVQEAKQKGYVTTLLHRRRYLPDITSRNFNLRSFAERTAINTPIQGSAADIIKKAMIDLSNRLKKENMKARMLLQVHDELILEAPKEEVERLQQIVPEVMENAVQLRVPLKVDYHFGPTWYDAK

SEQ ID NO: 156 - protein, 594 aa, synthetic construct
MGETEVALSDIDYTIVDEVTESILSDEAALVVEVLESNYHKAPILGFAIANEHGNFFIRTDTALSSSLFTQWLEDESKKKSVFDGKRAIVSLKWQGIQLRGVQFDLLIASYLLNPSQSTEDVASIAKTKQYTDVQSDEAIYGKGAKQKIPDEPVLAEHLVRKAAAIRALEQDFICDLQENEQYSLFTDLELPLSSILAEMEFTGVKIDVKRLKEMGEELTEQLKEIEQEIYELAGQEFNINSPKQLGVILFEKLQLPVLKKTKTGYSTSAEVLEKLAPQHEIVEKILHYRQLGKLQSTYIEGLLKVVHHDTNKVHTIFNQALTQTGRLSSTEPNLQNIPIRLEEGRKIRQTFVPSEPDWVIFSADYSQIELRVLAHIANDENLIDAFRHDLDIHTKTAMDIFHVNEDEVTPNMRRQAKAVNFGIVYGISDYGLSQNLNITRKEASEFIKRYFEIFPGVKQYMKDIVQEAKQKGYVTTLLHRRRYLPDITSRNFNLRSFAERTAINTPIQGSAADIIKKAMIDLSNRLKKENMKARMLLQVHDELILEAPKEEVERLQQIVPEVMENAVQLRVPLKVDYHFGPTWYDAKHHHHHH